SEQUENCE LISTING (11 sequences)

SEQ ID NO: 1 - protein, 468 aa, Clostridium saccharoperbutylacetonicum
MIKDTLVSITKDLKLKTNVENANLKNYKDDSSCFGVFENVENAISNAVHAQKILSLHYTKEQREKIITEIRKAALENKEILATMILEETHMGRYEDKILKHELVAKYTPGTEDLTTTAWSGDNGLTVVEMSPYGVIGAITPSTNPTETVICNSIGMIAAGNTVVFNGHPGAKKCVAFAVEMINKAIISCGGPENLVTTIKNPTMDSLDAIIKHPSIKLLCGTGGPGMVKTLLNSGKKAIGAGAGNPPVIVDDTADIEKAGKSIIEGCSFDNNLPCIAEKEVFVFENVADDLISNMLKNNAVIINEDQVSKLIDLVLQKNNETQEYSINKKWVGKDAKLFLDEIDVESPSSVKCIICEVSASHPFVMTELMMPILPIVRVKDIDEAIEYAKIAEQNRKHSAYIYSKNIDNLNRFEREIDTTIFVKNAKSFAGVGYEAEGFTTFTIAGSTGEGITSARNFTRQRRCVLAG

SEQ ID NO: 2 - DNA, 1407 bp, synthetic construct
  source 1..1407: mol_type = other DNA
atgattaaagacacgctggtttctatcaccaaagatttaaaattaaaaacaaatgttgaaaatgccaatctgaagaactacaaagatgattctagttgtttcggcgttttcgaaaatgttgaaaatgctatcagcaatgccgtacacgcacaaaagatattatcgctgcattatacaaaagaacaacgtgaaaaaatcatcactgagatacgtaaggccgcactggaaaataaagagattctggctacaatgattcttgaagaaacacatatgggacgttatgaagataaaatattaaagcatgaactggtagctaaatacactcctgggacagaagatttaactactactgcctggagcggcgataacgggctgacagttgtagaaatgtctccatatggcgttattggtgcaataactccttctaccaatccaactgaaactgtaatttgtaatagtattggcatgattgctgctggaaatactgtggtatttaacggacatccaggcgctaaaaaatgtgttgcttttgctgtcgaaatgatcaataaagctattattagctgtggtggtccggagaatctggtaacaactataaaaaatccaaccatggactctctggatgccattattaagcacccttcaataaaactgctttgcggaactggcgggccaggaatggtaaaaaccctgttaaattctggtaagaaagctattggtgctggtgctggaaatccaccagttattgtcgatgatactgctgatattgaaaaggctggtaagagtatcattgaaggctgttcttttgataataatttaccttgtattgcagaaaaagaagtatttgtttttgagaacgttgcagatgatttaatatctaacatgctgaaaaataatgctgtaattatcaatgaagatcaggtatcaaagttaatcgatttagtattacaaaaaaataatgaaactcaagaatactctattaataagaaatgggtcggtaaagatgcaaaattattcctcgatgaaatcgatgttgagtctccttcaagcgttaaatgcattatctgcgaagtcagtgcaagccatccatttgttatgacagaactgatgatgccaatattaccaattgtgcgcgttaaagatatcgatgaagctattgaatatgcaaaaattgcagaacaaaatagaaaacatagtgcctatatttattcaaaaaatatcgacaacctgaatcgctttgaacgtgaaatcgatactactatctttgtaaagaatgctaaatcttttgccggtgttggttatgaagcagaaggctttaccactttcactattgctggatccactggtgaaggcataacttctgcacgtaattttacccgccaacgtcgctgtgtactggccggttaa

SEQ ID NO: 3 - protein, 468 aa, synthetic construct
MIKDTLVSITKDLKLKTNVENANLKNYKDDSSCFGVFENVENAISNAVHAQKILSLHYTKEQREAMITEIRKAALENKEILATMILEETHMGRYEDKILKHELVAKYTPGTEDLTTTAWSGDNGLTVVEMSPYGVIGAITPSTNPTETVICNSIGMIAAGNTVVFNGHPGAKKSVAFAVEMINKAIISCGGPENLVTTIKNPTRDSLDAIIKHPSIKLLVGTGGPGMVKTLLNSGKKAIGAGQGNPPVIVDDTADIEKAGKSIIEGASFDNNLPCIAEKEVFVFENVADDLISNMLKNNAVIINEDQVSKLIDLVLQKNNETQEYSINKKWVGKDAKLFLDEIDVESPSSVKCIITEVSASHPFVMTELMMPILPIVRVKDIDEAIEYAKIAEQNHKHSAYIYSKNIDNLNRFEREIDTTIFVKNAKSFAGVGYEAPGFTTNTIAGSTGEGITSARNFTRQRRIVLVG

SEQ ID NO: 4 - DNA, 1407 bp, synthetic construct
  source 1..1407: mol_type = other DNA
atgattaaagacacgctggtttctatcaccaaagatttaaaattaaaaacaaatgttgaaaatgccaatctgaagaactacaaagatgattctagttgtttcggcgttttcgaaaatgttgaaaatgctatcagcaatgccgtacacgcacaaaagatattatcgctgcattatacaaaagaacaacgtgaagcgatgatcactgagatacgtaaggccgcactggaaaataaagagattctggctacaatgattcttgaagaaacacatatgggacgttatgaagataaaatattaaagcatgaactggtagctaaatacactcctgggacagaagatttaactactactgcctggagcggcgataacgggctgacagttgtagaaatgtctccatatggcgttattggtgcaataactccttctaccaatccaactgaaactgtaatttgtaatagtattggcatgattgctgctggaaatactgtggtatttaacggacatccaggcgctaaaaaatctgttgcttttgctgtcgaaatgatcaataaagctattattagctgtggtggtccggagaatctggtaacaactataaaaaatccaaccagggactctctggatgccattattaagcacccttcaataaaactgcttgttggaactggcgggccaggaatggtaaaaaccctgttaaattctggtaagaaagctattggtgctggtcagggaaatccaccagttattgtcgatgatactgctgatattgaaaaggctggtaagagtatcattgaaggcgcttcttttgataataatttaccttgtattgcagaaaaagaagtatttgtttttgagaacgttgcagatgatttaatatctaacatgctgaaaaataatgctgtaattatcaatgaagatcaggtatcaaagttaatcgatttagtattacaaaaaaataatgaaactcaagaatactctattaataagaaatgggtcggtaaagatgcaaaattattcctcgatgaaatcgatgttgagtctccttcaagcgttaaatgcattatcactgaagtcagtgcaagccatccatttgttatgacagaactgatgatgccaatattaccaattgtgcgcgttaaagatatcgatgaagctattgaatatgcaaaaattgcagaacaaaatcataaacatagtgcctatatttattcaaaaaatatcgacaacctgaatcgctttgaacgtgaaatcgatactactatctttgtaaagaatgctaaatcttttgccggtgttggttatgaagcaccgggctttaccactaatactattgctggatccactggtgaaggcataacttctgcacgtaattttacccgccaacgtcgcattgtactggtcggttaa

SEQ ID NO: 5 - protein, 468 aa, synthetic construct
  VARIANT 243: note = E
MIKDTLVSITKDLKLKTNVENANLKNYKDDSSCFGVFENVENAISNAVHAQKILSLHYTKEQREAMITEIRKAALENKEILATMILEETHMGRYEDKILKHELVAKYTPGTEDLTTTAWSGDNGLTVVEMSPYGVIGAITPSTNPTETVICNSIGMIAAGNTVVFNGHPGAKKSVAFAVEMINKAIISCGGPENLVTTIKNPTRDSLDAIIKHPSIKLLVGTGGPGMVKTLLNSGKKAIGAGEGNPPVIVDDTADIEKAGKSIIEGASFDNNLPCIAEKEVFVFENVADDLISNMLKNNAVIINEDQVSKLIDLVLQKNNETQEYSINKKWVGKDAKLFLDEIDVESPSSVKCIITEVSASHPFVMTELMMPILPIVRVKDIDEAIEYAKIAEQNHKHSAYIYSKNIDNLNRFEREIDTTIFVKNAKSFAGVGYEAPGFTTNTIAGSTGEGITSARNFTRQRRIVLVG

SEQ ID NO: 6 - protein, 468 aa, synthetic construct
  VARIANT 277: note = C
MIKDTLVSITKDLKLKTNVENANLKNYKDDSSCFGVFENVENAISNAVHAQKILSLHYTKEQREAMITEIRKAALENKEILATMILEETHMGRYEDKILKHELVAKYTPGTEDLTTTAWSGDNGLTVVEMSPYGVIGAITPSTNPTETVICNSIGMIAAGNTVVFNGHPGAKKSVAFAVEMINKAIISCGGPENLVTTIKNPTRDSLDAIIKHPSIKLLVGTGGPGMVKTLLNSGKKAIGAGQGNPPVIVDDTADIEKAGKSIIEGASFDNNLPCICEKEVFVFENVADDLISNMLKNNAVIINEDQVSKLIDLVLQKNNETQEYSINKKWVGKDAKLFLDEIDVESPSSVKCIITEVSASHPFVMTELMMPILPIVRVKDIDEAIEYAKIAEQNHKHSAYIYSKNIDNLNRFEREIDTTIFVKNAKSFAGVGYEAPGFTTNTIAGSTGEGITSARNFTRQRRIVLVG

SEQ ID NO: 7 - protein, 468 aa, synthetic construct
  VARIANT 435: note = X can be H or Q or M or R
MIKDTLVSITKDLKLKTNVENANLKNYKDDSSCFGVFENVENAISNAVHAQKILSLHYTKEQREAMITEIRKAALENKEILATMILEETHMGRYEDKILKHELVAKYTPGTEDLTTTAWSGDNGLTVVEMSPYGVIGAITPSTNPTETVICNSIGMIAAGNTVVFNGHPGAKKSVAFAVEMINKAIISCGGPENLVTTIKNPTRDSLDAIIKHPSIKLLVGTGGPGMVKTLLNSGKKAIGAGQGNPPVIVDDTADIEKAGKSIIEGASFDNNLPCIAEKEVFVFENVADDLISNMLKNNAVIINEDQVSKLIDLVLQKNNETQEYSINKKWVGKDAKLFLDEIDVESPSSVKCIITEVSASHPFVMTELMMPILPIVRVKDIDEAIEYAKIAEQNHKHSAYIYSKNIDNLNRFEREIDTTIFVKNAKSFAGVGYXAPGFTTNTIAGSTGEGITSARNFTRQRRIVLVG

SEQ ID NO: 8 - protein, 468 aa, synthetic construct
  VARIANT 142: note = X can be V or I
MIKDTLVSITKDLKLKTNVENANLKNYKDDSSCFGVFENVENAISNAVHAQKILSLHYTKEQREAMITEIRKAALENKEILATMILEETHMGRYEDKILKHELVAKYTPGTEDLTTTAWSGDNGLTVVEMSPYGVIGAITPXTNPTETVICNSIGMIAAGNTVVFNGHPGAKKSVAFAVEMINKAIISCGGPENLVTTIKNPTRDSLDAIIKHPSIKLLVGTGGPGMVKTLLNSGKKAIGAGQGNPPVIVDDTADIEKAGKSIIEGASFDNNLPCIAEKEVFVFENVADDLISNMLKNNAVIINEDQVSKLIDLVLQKNNETQEYSINKKWVGKDAKLFLDEIDVESPSSVKCIITEVSASHPFVMTELMMPILPIVRVKDIDEAIEYAKIAEQNHKHSAYIYSKNIDNLNRFEREIDTTIFVKNAKSFAGVGYEAPGFTTNTIAGSTGEGITSARNFTRQRRIVLVG

SEQ ID NO: 9 - protein, 468 aa, synthetic construct
  VARIANT 146: note = C
MIKDTLVSITKDLKLKTNVENANLKNYKDDSSCFGVFENVENAISNAVHAQKILSLHYTKEQREAMITEIRKAALENKEILATMILEETHMGRYEDKILKHELVAKYTPGTEDLTTTAWSGDNGLTVVEMSPYGVIGAITPSTNPCETVICNSIGMIAAGNTVVFNGHPGAKKSVAFAVEMINKAIISCGGPENLVTTIKNPTRDSLDAIIKHPSIKLLVGTGGPGMVKTLLNSGKKAIGAGQGNPPVIVDDTADIEKAGKSIIEGASFDNNLPCIAEKEVFVFENVADDLISNMLKNNAVIINEDQVSKLIDLVLQKNNETQEYSINKKWVGKDAKLFLDEIDVESPSSVKCIITEVSASHPFVMTELMMPILPIVRVKDIDEAIEYAKIAEQNHKHSAYIYSKNIDNLNRFEREIDTTIFVKNAKSFAGVGYEAPGFTTNTIAGSTGEGITSARNFTRQRRIVLVG

SEQ ID NO: 10 - protein, 468 aa, synthetic construct
  VARIANT 401: note = F
MIKDTLVSITKDLKLKTNVENANLKNYKDDSSCFGVFENVENAISNAVHAQKILSLHYTKEQREAMITEIRKAALENKEILATMILEETHMGRYEDKILKHELVAKYTPGTEDLTTTAWSGDNGLTVVEMSPYGVIGAITPSTNPTETVICNSIGMIAAGNTVVFNGHPGAKKSVAFAVEMINKAIISCGGPENLVTTIKNPTRDSLDAIIKHPSIKLLVGTGGPGMVKTLLNSGKKAIGAGQGNPPVIVDDTADIEKAGKSIIEGASFDNNLPCIAEKEVFVFENVADDLISNMLKNNAVIINEDQVSKLIDLVLQKNNETQEYSINKKWVGKDAKLFLDEIDVESPSSVKCIITEVSASHPFVMTELMMPILPIVRVKDIDEAIEYAKIAEQNHKHSAFIYSKNIDNLNRFEREIDTTIFVKNAKSFAGVGYEAPGFTTNTIAGSTGEGITSARNFTRQRRIVLVG

SEQ ID NO: 11 - protein, 468 aa, synthetic construct
  VARIANT 442: note = F
MIKDTLVSITKDLKLKTNVENANLKNYKDDSSCFGVFENVENAISNAVHAQKILSLHYTKEQREAMITEIRKAALENKEILATMILEETHMGRYEDKILKHELVAKYTPGTEDLTTTAWSGDNGLTVVEMSPYGVIGAITPSTNPTETVICNSIGMIAAGNTVVFNGHPGAKKSVAFAVEMINKAIISCGGPENLVTTIKNPTRDSLDAIIKHPSIKLLVGTGGPGMVKTLLNSGKKAIGAGQGNPPVIVDDTADIEKAGKSIIEGASFDNNLPCIAEKEVFVFENVADDLISNMLKNNAVIINEDQVSKLIDLVLQKNNETQEYSINKKWVGKDAKLFLDEIDVESPSSVKCIITEVSASHPFVMTELMMPILPIVRVKDIDEAIEYAKIAEQNHKHSAYIYSKNIDNLNRFEREIDTTIFVKNAKSFAGVGYEAPGFTTFTIAGSTGEGITSARNFTRQRRIVLVG

(19) United States
(12) Patent Application Publication
(10) Pub. No.: US 2025/0368968 A1
(43) Pub. Date: Dec. 4, 2025

(54) ALDEHYDE DEHYDROGENASE VARIANTS AND METHODS OF USE

(71) Applicant: Genomatica, Inc., San Diego, CA (US)

(72) Inventors: Amit Mahendra Shah, San Diego, CA (US); Justin Robert Colquitt, San Diego, CA (US); Joseph Roy Warner, Oceanside, CA (US); Deqiang Zhang, San Diego, CA (US)

(21) Appl. No.: 18/702,714

(22) PCT Filed: Oct. 18, 2022
(86) PCT No.: PCT/US2022/078308

§ 371 Date: Apr. 18, 2024

Related U.S. Application Data
(60) Provisional application No. 63/257,743, filed on Oct. 20, 2021.

(51) Int. Cl. C12N 9/02; C12N 1/20; C12P 7/18; C12P 7/24; C12P 13/02; C12R 1/19

(52) U.S. Cl. CPC C12N 9/0008; C12N 1/20; C12P 7/18; C12P 7/24; C12P 13/02; C12R 2001/19; C12Y 102/01003

(57) ABSTRACT

The disclosure provides polypeptides and encoding nucleic acids of engineered aldehyde dehydrogenases. The disclosure also provides cells expressing an engineered form of the aldehyde dehydrogenase. The disclosure further provides methods for producing a bioderived compound, such as 3-hydroxybutyraldehyde, 1,3-butanediol, 4-hydroxybutyraldehyde, 1,4-butanediol, comprising culturing cells expressing an engineered aldehyde dehydrogenase.

### CROSS-REFERENCE TO RELATED APPLICATIONS

This application claims the benefit of U.S. Provisional Patent Application No. 63/257,743, filed Oct. 20, 2021, the entire contents of which are incorporated by reference herein.

### REFERENCE TO SEQUENCE LISTING SUBMITTED ELECTRONICALLY

The instant application contains a Sequence Listing, which has been submitted via Patent Center. The Sequence Listing titled 199683-121001_PCT.xml, which was created on Oct. 18, 2022 and is 20,480 bytes in size, is hereby incorporated by reference in its entirety.

### FIELD OF THE INVENTION

The present disclosure relates generally to aldehyde dehydrogenase variants and methods of using such variants, and more specifically to aldehyde dehydrogenase variants encoded by recombinant nucleic acids that have been introduced to a non-naturally occurring microbial organism to produce a bioderived compound such as 3-hydroxybutyraldehyde, 1,3-butanediol, 4-hydroxybutyraldehyde, and 1,4-butanediol, and products derived therefrom.

### BACKGROUND OF THE INVENTION

Various commodity chemicals are used to make desired products for commercial use. Many of the commodity chemicals are derived from petroleum. Such commodity chemicals have various uses, including use as solvents, resins, polymer precursors, and specialty chemicals. Desired commodity chemicals include 4-carbon molecules such as 1,4-butanediol and 1,3-butanediol, upstream precursors and downstream products.

1,3-butanediol (1,3-BDO; also referred to as 1,3-butylene glycol, 1,3-BG, butylene glycol, BG) is traditionally produced from acetylene via its hydration. The resulting acetaldehyde is then converted to 3-hydroxybutyraldehdye, which is subsequently reduced to form 1,3-BDO. More recently, acetylene has been replaced by the less expensive ethylene as a source of acetaldehyde. 1,3-BDO is commonly used as an organic solvent for food flavoring agents. It is also used as a co-monomer for polyurethane and polyester resins and is widely employed as a hypoglycemic agent. Optically active 1,3-BDO is a useful starting material for the synthesis of biologically active compounds and liquid crystals. Another use of 1,3-BDO is that its dehydration affords 1,3-butadiene (Ichikawa et al., Journal of Molecular Catalysis A-Chemical, 256:106-112 (2006); Ichikawa et al., Journal of Molecular Catalysis A-Chemical, 231:181-189 (2005), which is useful in the manufacture synthetic rubbers (e.g., tires), latex, and resins. The reliance on petroleum based feedstocks for either acetylene or ethylene warrants the development of a renewable feedstock based route to 1,3-BDO and to butadiene.

1,3-BDO has further food related uses including use directly as a food source, a food ingredient, a flavoring agent, a solvent or solubilizer for flavoring agents, a stabilizer, an emulsifier, and an anti-microbial agent and preservative. 1,3-BDO is used in the pharmaceutical industry as a parenteral drug solvent. 1,3-BDO finds use in cosmetics as an ingredient that is an emollient, a humectant, that prevents crystallization of insoluble ingredients, a solubilizer for less-water-soluble ingredients such as fragrances, and as an anti-microbial agent and preservative. For example, it can be used as a humectant, especially in hair sprays and setting lotions; it reduces loss of aromas from essential oils, preserves against spoilage by microorganisms, and is used as a solvent for benzoates. 1,3-BDO can be use at concentrations from 0.1 percent or less to 50 percent or greater. It is used in hair and bath products, eye and facial makeup, fragrances, personal cleanliness products, and shaving and skin care preparations (see, e.g., the Cosmetic Ingredient Review board's report: “Final Report on the Safety Assessment of Butylene Glycol, Hexylene Glycol, Ethoxydiglycol, and Dipropylene Glycol”, Journal of the American College of Toxicology, Volume 4, Number 5, 1985, which is incorporated herein by reference). This report provides specific uses and concentrations of 1,3-BDO (butylene glycol) in cosmetics; see for examples the report's Table 2 therein entitled “Product Formulation Data”.

1,4-butanediol (1,4-BDO) is a valuable chemical for the production of high performance polymers, solvents, and fine chemicals. It is the basis for producing other high value chemicals such as tetrahydrofuran (THF) and gamma-butyrolactone (GBL). The value chain is comprised of three main segments including: (1) polymers, (2) THF derivatives, and (3) GBL derivatives. In the case of polymers, 1,4-BDO is a comonomer for polybutylene terephthalate (PBT) production. PBT is a medium performance engineering thermoplastic used in automotive, electrical, water systems, and small appliance applications. Conversion to THF, and subsequently to polytetramethylene ether glycol (PTMEG), provides an intermediate used to manufacture spandex products such as LYCRA® fibers. PTMEG is also combined with 1,4-BDO in the production of specialty polyester ethers (COPE). COPEs are high modulus elastomers with excellent mechanical properties and oil/environmental resistance, allowing them to operate at high and low temperature extremes. PTMEG and 1,4-BDO also make thermoplastic polyurethanes processed on standard thermoplastic extrusion, calendaring, and molding equipment, and are characterized by their outstanding toughness and abrasion resistance. The GBL produced from 1,4-BDO provides the feedstock for making pyrrolidones, as well as serving the agrochemical market. The pyrrolidones are used as high performance solvents for extraction processes of increasing use, including for example, in the electronics industry and in pharmaceutical production.

1,4-BDO is produced by two main petrochemical routes with a few additional routes also in commercial operation. One route involves reacting acetylene with formaldehyde, followed by hydrogenation. More recently, 1,4-BDO processes involving butane or butadiene oxidation to maleic anhydride, followed by hydrogenation have been introduced. 1,4-BDO is used almost exclusively as an intermediate to synthesize other chemicals and polymers.

It is desirable to develop methods for production of commodity chemicals to provide renewable sources for petroleum-based products and to provide less energy- and capital-intensive processes. Thus, there exists a need for methods that facilitate production of desired products. The present invention satisfies this need and provides related advantages as well.

### SUMMARY OF INVENTION

In some embodiments, provided herein is an engineered aldehyde dehydrogenase that is a variant of SEQ ID NO: 3 or a functional fragment thereof. Such an engineered aldehyde dehydrogenase includes one or more alterations at a position described in TABLE 2. An engineered aldehyde dehydrogenase described herein, in some embodiments, is capable of catalyzing the conversion of 3-hydroxybutyryl-CoA to 3-hydroxybutyraldehyde. In some embodiments, the engineered aldehyde dehydrogenase has: 1) higher specificity for conversion of 3-hydroxybutyryl-CoA to 3-hydroxybutyraldehyde over conversion of acetyl-CoA to acetaldehyde; or 2) higher specificity for conversion of (R)-3-hydroxybutyryl-CoA to (R)-3-hydroxybutyraldehyde over conversion of (S)-3-hydroxybutyryl-CoA to (S)-3-hydroxybutyraldehyde. An engineered aldehyde dehydrogenase described herein, in some embodiments, is capable of catalyzing the conversion of 4-hydroxybutyryl-CoA to 4-hydroxybutyraldehyde. In some embodiments, the engineered aldehyde dehydrogenase has higher specificity for conversion of 4-hydroxybutyryl-CoA to 4-hydroxybutyraldehyde over conversion of acetyl-CoA to acetaldehyde.

In some embodiments, an engineered aldehyde dehydrogenase described herein has activity that is at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, or at least 200% higher than the activity of an aldehyde dehydrogenase consisting of the amino acid sequence of SEQ ID NO: 3.

In some embodiments, an engineered aldehyde dehydrogenase described herein includes one or more amino acid alterations at a position corresponding to position 142, 243, 277, 401, 435, or 442, or a combination thereof, in SEQ ID NO: 3.

In some embodiments, an engineered aldehyde dehydrogenase described herein includes one or more amino acid alterations at a position corresponding to position 435 or 442, or a combination thereof, in SEQ ID NO: 3.

In some embodiments, an engineered aldehyde dehydrogenase described herein includes one or more alterations at a position described in TABLE 2, wherein the one or more amino acid alterations are conservative amino acid substitutions.

In some embodiments, an engineered aldehyde dehydrogenase described herein includes one or more alterations at a position described in TABLE 2, wherein the one or more amino acid alterations are non-conservative amino acid substitutions.

In some embodiments, the engineered aldehyde dehydrogenase described herein includes one or more alterations at a position described in TABLE 2, wherein the one or more amino acid alterations result in an engineered aldehyde dehydrogenase having a specific alteration as described in TABLE 2, including, in some embodiments, a specific alteration or combination of alterations that results in a particular improvement in activity as described in TABLE 2 (e.g., 1,3-BDO production).

In some embodiments, the engineered aldehyde dehydrogenase described herein includes one or more alterations at a position described in TABLE 2, wherein the one or more amino acid alterations includes at least 2, 3, 4, 5, 6, or 7 alterations.

In some embodiments, the engineered aldehyde dehydrogenase described herein includes one or more alterations at a position described in TABLE 2, wherein the amino acid sequence, other than the one or more amino acid alterations, has at least 65%, 70%, 75%, 80%, 85%, 90%, 95%, 98%, 99% sequence identity, or is identical, to the amino acid sequence referenced in SEQ ID NO: 3.

In some embodiments, provided herein is a recombinant nucleic acid encoding an engineered aldehyde dehydrogenase described herein. In some embodiments, such a recombinant nucleic acid has a nucleotide sequence encoding the engineered aldehyde dehydrogenase operatively linked to a promoter. In some embodiments, also provided herein is a vector having such recombinant nucleic.

In some embodiments, provided herein is a non-naturally occurring microbial organism having a recombinant nucleic acid encoding an engineered aldehyde dehydrogenase described herein. Such a microbial organism, in some embodiments, further includes a pathway that produces 3-hydroxybutyraldehyde and/or 1,3-butanediol, or an ester or amide thereof. A microbial organism having such a pathway, in some embodiments, is capable of producing at least 10% more 3-hydroxybutyraldehyde and/or 1,3-butanediol, or an ester or amide thereof compared to a control microbial organism that does not comprise the recombinant nucleic acid encoding an engineered aldehyde dehydrogenase described herein. Alternatively, in some embodiments, such a microbial organism further includes a pathway that produces 4-hydroxybutyraldehyde and/or 1,4-butanediol, or an ester or amide thereof. A microbial organism having such a pathway, in some embodiments, is capable of producing at least 10% more 4-hydroxybutyraldehyde and/or 1,4-butanediol, or an ester or amide thereof compared to a control microbial organism that does not comprise the recombinant nucleic acid encoding an engineered aldehyde dehydrogenase described herein. In some embodiments, the one or more enzymes of such pathways are encoded by an exogenous nucleic acid.

In some embodiments, a microbial organism described herein includes an exogenous nucleic acid that is heterologous to the microbial organism. In some embodiments, a microbial organism described herein includes an exogenous nucleic acid that is homologous to the microbial organism.

In some embodiments, a microbial organism described herein produces a decreased amount of a by-product as compared to a control microbial organism that does not include the recombinant nucleic acid encoding an engineered aldehyde dehydrogenase described herein. Such a microbial organism, in some embodiments, produces a decreased amount of ethanol and/or 4-hydroxy-2-butanone. In some embodiments, such a microbial organism provided herein is capable of producing at least 10% less by-product compared to a control microbial organism that does not comprise the recombinant nucleic acid encoding an engineered aldehyde dehydrogenase described herein.

In some embodiments, a microbial organism described herein is in a substantially anaerobic culture medium.

In some embodiments, a microbial organism described herein is a species of bacteria, yeast, or fungus.

In some embodiments, provided herein is a method for producing 3-hydroxybutyraldehyde and/or 1,3-butanediol, or an ester or amide thereof. Such a method can include culturing a non-naturally occurring microbial organism described herein under conditions and for a sufficient period of time to produce the 3-hydroxybutyraldehyde and/or 1,3-butanediol, or an ester or amide thereof. In some embodiments, such a method further includes separating the 3-hydroxybutyraldehyde and/or 1,3-butanediol, or an ester or amide thereof from other components in the culture. Methods for performing such separating includes extraction, continuous liquid-liquid extraction, pervaporation, membrane filtration, membrane separation, reverse osmosis, electrodialysis, distillation, crystallization, centrifugation, extractive filtration, ion exchange chromatography, absorption chromatography, or ultrafiltration.

In some embodiments, provided herein is culture medium having the 3-hydroxybutyraldehyde and/or 1,3-butanediol, or an ester or amide thereof produced by a method provided herein, wherein the 3-hydroxybutyraldehyde and/or 1,3-butanediol, or an ester or amide thereof has a carbon-12, carbon-13 and carbon-14 isotope ratio that reflects an atmospheric carbon dioxide uptake source.

In some embodiments, provided herein is a 3-hydroxybutyraldehyde and/or 1,3-butanediol, or an ester or amide thereof produced according to a method described herein. Such a 3-hydroxybutyraldehyde and/or 1,3-butanediol, or an ester or amide, in some embodiments, has an Fm value of at least 80%, at least 85%, at least 90%, at least 95% or at least 98%.

In some embodiments, provided herein is a composition the 3-hydroxybutyraldehyde and/or 1,3-butanediol, or an ester or amide thereof described herein and a compound other than the 3-hydroxybutyraldehyde and/or 1,3-butanediol, or an ester or amide thereof. In some embodiments, the compound other than the 3-hydroxybutyraldehyde and/or 1,3-butanediol, or an ester or amide thereof is a trace amount of a cellular portion of a non-naturally occurring microbial organism having a pathway that produces 3-hydroxybutyraldehyde and/or 1,3-butanediol, or an ester or amide thereof. In some embodiments, provided herein is composition having the 3-hydroxybutyraldehyde and/or 1,3-butanediol, or an ester or amide thereof described herein, or a cell lysate or culture supernatant thereof.

In some embodiments, provided herein is a method for producing 4-hydroxybutyraldehyde and/or 1,4-butanediol, or an ester or amide thereof. Such a method can include culturing a non-naturally occurring microbial organism described herein under conditions and for a sufficient period of time to produce 4-hydroxybutyraldehyde and/or 1,4-butanediol, or an ester or amide thereof. In some embodiments, such a method further includes separating the 4-hydroxybutyraldehyde and/or 1,4-butanediol, or an ester or amide thereof from other components in the culture. Methods for performing such separating includes extraction, continuous liquid-liquid extraction, pervaporation, membrane filtration, membrane separation, reverse osmosis, electrodialysis, distillation, crystallization, centrifugation, extractive filtration, ion exchange chromatography, absorption chromatography, or ultrafiltration.

In some embodiments, provided herein is culture medium having the 4-hydroxybutyraldehyde and/or 1,4-butanediol, or an ester or amide thereof produced by a method provided herein, wherein the 4-hydroxybutyraldehyde and/or 1,4-butanediol, or an ester or amide thereof has a carbon-12, carbon-13 and carbon-14 isotope ratio that reflects an atmospheric carbon dioxide uptake source.

In some embodiments, provided herein is a 4-hydroxybutyraldehyde and/or 1,4-butanediol, or an ester or amide thereof produced according to a method described herein. Such a 4-hydroxybutyraldehyde and/or 1,4-butanediol, or an ester or amide, in some embodiments, has an Fm value of at least 80%, at least 85%, at least 90%, at least 95% or at least 98%.

In some embodiments, provided herein is a composition the 4-hydroxybutyraldehyde and/or 1,4-butanediol, or an ester or amide thereof described herein and a compound other than the 4-hydroxybutyraldehyde and/or 1,4-butanediol, or an ester or amide thereof. In some embodiments, the compound other than the 4-hydroxybutyraldehyde and/or 1,4-butanediol, or an ester or amide thereof is a trace amount of a cellular portion of a non-naturally occurring microbial organism having a pathway that produces 4-hydroxybutyraldehyde and/or 1,4-butanediol, or an ester or amide thereof. In some embodiments, provided herein is composition having the 4-hydroxybutyraldehyde and/or 1,4-butanediol, or an ester or amide thereof described herein, or a cell lysate or culture supernatant thereof.

In some embodiments, provided herein is the use of an engineered aldehyde dehydrogenase described herein as a biocatalyst.

In some embodiments, provided herein is a composition having the engineered aldehyde dehydrogenase described herein and at least one substrate for the engineered aldehyde dehydrogenase. As such, in some embodiments, the engineered aldehyde dehydrogenase can react with the substrate under in vitro conditions. In some embodiments, the substrate is a specific compound, such as 3-hydroxybutyryl-CoA, (R)-3-hydroxybutyryl-CoA, or 4-hydroxybutyryl-CoA.

### DETAILED DESCRIPTION OF THE INVENTION

The subject matter described herein relates to enzyme variants that have desirable properties and are useful for producing desired products (e.g., 3-hydroxybutyraldehyde, especially (R)-3-hydroxybutyraldehyde, 4-hydroxybutyraldehyde, 1,3-butanediol, 1,4-butanediol, or an ester or amide of 1,3-butanediol or 1,4-butanediol). In some embodiments, the subject matter described herein relates to engineered aldehyde dehydrogenases, which are enzyme variants that have markedly different structural and/or functional characteristics compared to a wild-type aldehyde dehydrogenase that occurs in nature. Thus, the engineered aldehyde dehydrogenases provided herein are not naturally occurring enzymes. Such engineered aldehyde dehydrogenases provided are useful in an engineered cell, such as a microbial organism, that has been engineered to produce a desired product (e.g., 3-hydroxybutyraldehyde, especially (R)-3-hydroxybutyraldehyde, 4-hydroxybutyraldehyde, 1,3-butanediol, 1,4-butanediol, or an ester or amide of 1,3-butanediol or 1,4-butanediol). For example, as disclosed herein, a cell, such as a microbial organism, having a metabolic pathway can produce a desired product (e.g., 3-hydroxybutyraldehyde, especially (R)-3-hydroxybutyraldehyde, 4-hydroxybutyraldehyde, 1,3-butanediol, 1,4-butanediol, or an ester or amide of 1,3-butanediol or 1,4-butanediol). Engineered aldehyde dehydrogenases having desirable characteristics as described herein can be introduced into a cell, such as microbial organism, that has a metabolic pathway that uses aldehyde dehydrogenase activity to produce a desired product (e.g., 3-hydroxybutyraldehyde, especially (R)-3-hydroxybutyraldehyde, 4-hydroxybutyraldehyde, 1,3-butanediol, 1,4-butanediol, or an ester or amide of 1,3-butanediol or 1,4-butanediol). Thus, the engineered aldehyde dehydrogenases provided herein can be utilized in engineered cells, such as microbial organisms, to produce a desired product. Such engineered aldehyde dehydrogenases are additionally useful as biocatalysts for carrying out desired reactions in vitro. Thus, the engineered aldehyde dehydrogenase provided herein can be utilized in engineered cells, such as microbial organisms, to produce a desired product or as an in vitro biocatalyst to produce a desired product.

### Conventions and Abbreviations

| Abbreviation | Convention |
|---|---|
| Ala; A | Alanine |
| Arg; R | Arginine |
| Asn; N | Asparagine |
| Asp; D | Aspartic acid |
| Cys; C | Cysteine |
| Glu; E | Glutamic acid |
| Gln; Q | Glutamine |
| Gly; G | Glycine |
| His; H | Histidine |
| Ile; I | Isoleucine |
| Leu; L | Leucine |
| Lys; K | Lysine |
| Met; M | Methionine |
| Phe; F | Phenylalanine |
| Pro; P | Proline |
| Ser; S | Serine |
| Thr; T | Threonine |
| Trp; W | Tryptophan |
| Tyr; Y | Tyrosine |
| Val; V | Valine |
| 1,3-BDO | 1,3-butanediol |
| 1,4-BDO | 1,4-butanediol |
| 3-HB | 3-hydroxybutyrate |
| 3-HBal | 3-hydroxybutyraldehyde |
| 3-HB-CoA | 3-hydroxybutyryl-CoA |
| 4-HB | 4-hydroxybutyrate |
| 4-HBal | 4-hydroxybutyraldehyde |
| 4-HB-CoA | 4-hydroxybutyryl-CoA |
| 4-OH-2-But | 4-hydroxy-2-butanone |
| AcCoA | acetyl-CoA |
| EtOH | ethanol |
| R-3-HB | (R)-3-hydroxybutyrate |
| R-3-HBal | (R)-3-hydroxybutyraldehyde |
| R-3-HB-CoA | (R)-3-hydroxybutyryl-CoA |
| S-3-HB | (S)-3-hydroxybutyrate |
| S-3-HBal | (S)-3-hydroxybutyraldehyde |
| S-3-HB-CoA | (S)-3-hydroxybutyryl-CoA |

As used herein the term “about” means±1000 of the stated value. The term “about” can mean rounded to the nearest significant digit. Thus, about 50% means 4.500 to 5.500. Additionality, about in reference to a specific number also includes that exact number. For example, about 500 also includes exact 500.

As used herein, the term “alteration” or grammatical equivalents thereof when used in reference to any peptide, polypeptide, protein, nucleic acid or polynucleotide described herein refers to a change in structure of an amino acid residue or nucleic acid base relative to the starting or reference residue or base. An alteration of an amino acid residue includes, for example, deletions, insertions and substituting one amino acid residue for a structurally different amino acid residue. Such substitutions can be a conservative substitution, a non-conservative substitution, a substitution to a specific sub-class of amino acids, or a combination thereof as described herein. An alteration of a nucleic acid base includes, for example, changing one naturally occurring base for a different naturally occurring base, such as changing an adenine to a thymine or a guanine to a cytosine or an adenine to a cytosine or a guanine to a thymine. An alteration of a nucleic acid base may result in an alteration of the encoding peptide, polypeptide or protein by changing the encoded amino acid residue or function of the peptide, polypeptide or protein. An alteration of a nucleic acid base may not result in an alteration of the amino acid sequence or function of encoded peptide, polypeptide or protein, also known as a silent mutation.

As used herein, the term “bioderived” means derived from or synthesized by a biological organism and can be considered a renewable resource since it can be generated by a biological organism. Such a biological organism, in particular the non-naturally occurring microbial organism disclosed herein, can utilize feedstock or biomass, such as, sugars (e.g., cellobiose, glucose, fructose, xylose, galactose (e.g., galactose from marine plant biomass), and sucrose), carbohydrates obtained from an agricultural, plant, bacterial, or animal source, and glycerol (e.g., crude glycerol by-product from biodiesel manufacturing) for synthesis of a desired bioderived compound.

As used herein, the term “conservative substitution” refers to the replacement of one amino acid for another such that the replacement takes place within a family of amino acids that are related in their side chains. Alternatively, the term “non-conservative substitution” refers to the replacement of one amino acid residue for another such that the replaced residue is going from one family of amino acids to a different family of residues. Genetically encoded amino acids can be divided into four families: (1) acidic (negatively charged)=Asp (D), Glu (G); (2) basic (positively charged)=Lys (K), Arg (R), His (H); (3) non-polar (hydrophobic)=Cys (C), Ala (A), Val (V), Leu (L), Ile (I), Pro (P), Phe (F), Met (M), Trp (W), Gly (G), Tyr (Y), with non-polar also being subdivided into: (i) strongly hydrophobic=Ala (A), Val (V), Leu (L), Ile (I), Met (M), Phe (F); and (ii) moderately hydrophobic=Gly (G), Pro (P), Cys (C), Tyr (Y), Trp (W); and (4) uncharged polar=Asn (N), Gln (Q), Ser (S), Thr (T). In alternative fashion, the amino acid repertoire can be grouped as (1) acidic (negatively charged)=Asp (D), Glu (G); (2) basic (positively charged)=Lys (K), Arg (R), His (H), and (3) aliphatic=Gly (G), Ala (A), Val (V), Leu (L), Ile (I), Ser (S), Thr (T), with Ser (S) and Thr (T) optionally being grouped separately as aliphatic-hydroxyl; (4) aromatic=Phe (F), Tyr (Y), Trp (W); (5) amide=Asn (N), Glu (Q); and (6) sulfur-containing=Cys (C) and Met (M) (see, for example, Biochemistry, 4th ed., Ed. by L. Stryer, WH Freeman and Co., 1995, which is incorporated by reference herein in its entirety).

As used herein, the term “culture medium,” “medium,” “growth medium” or grammatical equivalents thereof refers to a liquid or solid (e.g., gelatinous) substance containing nutrients that support the growth of a cell, including a microbial organism, such as the microbial organism described herein. Nutrients that support growth include, but are not limited to, the following: a substrate that supplies carbon, such as, but are not limited to, cellobiose, galactose, glucose, xylose, ethanol, acetate, arabinose, arabitol, sorbitol and glycerol; salts that provide essential elements including magnesium, nitrogen, phosphorus, and sulfur; a source for amino acids, such as peptone or tryptone; and a source for vitamin content, such as yeast extract. Culture medium can be a defined medium, in which quantities of all ingredients are known, or an undefined medium, in which the quantities of all ingredients are not known. Culture medium can also include substances other than nutrients needed for growth, such as a substance that only allows select cells to grow (e.g., antibiotic or antifungal), which are generally found in selective medium, or a substance that allows for differentiation of one microbial organism over another when grown on the same medium, which are generally found in differential or indicator medium. Such substances are well known to a person skilled in the art.

As used herein, the term “engineered” or “variant” when used in reference to any peptide, polypeptide, protein, nucleic acid or polynucleotide described herein refers to a sequence of amino acids or nucleic acids having at least one alteration at an amino acid residue or nucleic acid base as compared to a parent sequence. Such a sequence of amino acids or nucleic acids is not naturally occurring. The parent sequence of amino acids or nucleic acids can be, for example, a wild-type sequence or a homolog thereof, or a modified variant of a wild-type sequence or homolog thereof.

“Exogenous” as it is used herein is intended to mean that the referenced molecule or the referenced activity is introduced into the host microbial organism. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the microbial organism. When used in reference to a biosynthetic activity, the term refers to an activity that is introduced into the host reference organism. The source can be, for example, a homologous or heterologous encoding nucleic acid that expresses the referenced activity following introduction into the host microbial organism. Therefore, the term “endogenous” refers to a referenced molecule or activity that is present in the host. Similarly, the term when used in reference to expression of an encoding nucleic acid refers to expression of an encoding nucleic acid contained within the microbial organism. The term “heterologous” refers to a molecule or activity derived from a source other than the referenced species whereas “homologous” refers to a molecule or activity derived from the host microbial organism. Accordingly, exogenous expression of an encoding nucleic acid described herein can utilize either or both a heterologous or homologous encoding nucleic acid.

It is understood that, when more than one recombinant nucleic acid and/or exogenous nucleic acid is included into a microbial organism, the more than one recombinant nucleic acid and/or exogenous nucleic acid refers to the referenced encoding nucleic acid or biosynthetic activity, as discussed herein. It is further understood, as disclosed herein, that such more than one recombinant nucleic acids or exogenous nucleic acids can be introduced into the host microbial organism on separate nucleic acid molecules, on polycistronic nucleic acid molecules, or a combination thereof, and still be considered as more than one recombinant nucleic acid and/or exogenous nucleic acid. For example, as disclosed herein a microbial organism can be engineered to express two or more recombinant and/or exogenous nucleic acids encoding a desired pathway enzyme or protein. In the case where two recombinant and/or exogenous nucleic acids encoding an enzyme or protein having a desired activity are introduced into a host microbial organism, it is understood that the two recombinant and/or exogenous nucleic acids can be introduced as a single nucleic acid, for example, on a single plasmid, on separate plasmids, can be integrated into the host chromosome at a single site or multiple sites, and still be considered as two exogenous nucleic acids. Similarly, it is understood that more than two recombinant and/or exogenous nucleic acids can be introduced into a host organism in any desired combination, for example, on a single plasmid, on separate plasmids, can be integrated into the host chromosome at a single site or multiple sites, and still be considered as two or more recombinant or exogenous nucleic acids, for example three exogenous nucleic acids. Thus, the number of referenced recombinant or exogenous nucleic acids or biosynthetic activities refers to the number of encoding nucleic acids or the number of biosynthetic activities, not the number of separate nucleic acids introduced into the host organism.

The term “Fm value” or “Fraction Modern value” when used in reference to a compound is a ratio of carbon-14 (¹⁴C) to carbon-12 (¹²C). Specifically, Fm value is computed from the expression: Fm=(S−B)/(M−B), where B, S and M represent the ¹⁴C/¹²C ratios of the blank, the sample and the modern reference, respectively. Fm value is a measurement of the deviation of the ¹⁴C/¹²C ratio of a sample from “Modern.” Modern is defined as 95% of the radiocarbon concentration (in AD 1950) of National Bureau of Standards (NBS) Oxalic Acid I (i.e., standard reference materials (SRM) 4990b) normalized to δ¹³C_{VPDB}=−19 per mil (Olsson, The use of Oxalic acid as a Standard. in, Radiocarbon Variations and Absolute Chronology, Nobel Symposium, 12th Proc., John Wiley & Sons, New York (1970)). Mass spectrometry results, for example, measured by ASM, are calculated using the internationally agreed upon definition of 0.95 times the specific activity of NBS Oxalic Acid I (SRM 4990b) normalized to δ¹³C_{VPDB}=−19 per mil. This is equivalent to an absolute (AD 1950) ¹⁴C/¹²C ratio of 1.176±0.010×10⁻¹² (Karlen et al., Arkiv Geofysik, 4:465-471 (1968)). The standard calculations take into account the differential uptake of one isotope with respect to another, for example, the preferential uptake in biological systems of C¹² over C¹³ over C¹⁴, and these corrections are reflected as a Fm corrected for δ¹³. An Fm=0% represents the entire lack of carbon-14 atoms in a material, thus indicating a fossil (for example, petroleum based) carbon source, whereas a Fm=100%, after correction for the post-1950 injection of carbon-14 into the atmosphere from nuclear bomb testing, indicates an entirely modern carbon source. The percent modern carbon (pMC) can be greater than 100% because of the continuing but diminishing effects of the 1950s nuclear testing programs, which resulted in a considerable enrichment of carbon-14 in the atmosphere. Because all sample carbon-14 activities are referenced to a “pre-bomb” standard, and because nearly all new biobased products are produced in a post-bomb environment, all pMC values (after correction for isotopic fraction) must be multiplied by 0.95 (as of 2010) to better reflect the true biobased content of the sample. A biobased content that is greater than 103% suggests that either an analytical error has occurred, or that the source of biobased carbon is more than several years old. Applications of carbon-14 dating techniques to quantify bio-based content of materials are well known in the art (see, e.g., Currie et al., Nuclear Instruments and Methods in Physics Research B, 172:281-287 (2000), and Colonna et al., Green Chemistry, 13:2543-2548 (2011)).

As used herein, the term “functional fragment” when used in reference to a peptide, polypeptide or protein is intended to refer to a portion of the peptide, polypeptide or protein that retains some or all of the activity (e.g., catalyzing the conversion of 3-hydroxybutyryl-CoA to 3-hydroxybutyraldehyde or 4-hydroxybutyryl-CoA to 4-hydroxybutyraldehyde) of the original peptide, polypeptide or protein from which the fragment was derived. Such functional fragments include amino acid sequences that are about 200 to about 460, about 200 to about 450, about 200 to about 440, about 200 to about 430, about 200 to about 420, about 200 to about 410, about 200 to about 400, about 200 to about 390, about 200 to about 380, about 200 to about 370, about 200 to about 360, about 200 to about 350, about 300 to about 460, about 300 to about 450, about 300 to about 440, about 300 to about 430, about 300 to about 420, about 300 to about 410, about 300 to about 400, about 300 to about 390, about 300 to about 380, about 300 to about 370, about 300 to about 350, about 300 to about 340, about 300 to about 330, about 300 to about 320, about 300 to about 310, about 400 to about 460, about 400 to about 450, about 400 to about 440, about 400 to about 430, about 400 to about 420, about 400 to about 410, about 450 to about 460 amino acids in length. These functional fragments can, for example, be truncations (e.g., C-terminal or N-terminal truncations) of a peptide, polypeptide, or protein. Functional fragments can also include one or more amino acid alteration described herein, such as an amino acid alteration of an engineered peptide described herein.

As used herein, the term “isolated” when used in reference to a molecule (e.g., peptide, polypeptide, protein, nucleic acid, polynucleotide, vector) or a cell (e.g., a yeast cell) refers to a molecule or cell that is substantially free of at least one component with which the referenced molecule or cell is found in nature. The term includes a molecule or cell that is removed from some or all components with which it is found in its natural environment. Therefore, an isolated molecule or cell can be partly or completely separated from other substances with which it is found in nature or with which it is grown, stored or subsisted in non-naturally occurring environments.

As used herein, the terms “microbial,” “microbial organism” or “microorganism” are intended to mean any organism that exists as a microscopic cell that is included within the domains of archaea, bacteria or eukarya. Therefore, the term is intended to encompass prokaryotic or eukaryotic cells or organisms having a microscopic size and includes bacteria, archaea and eubacteria of all species as well as eukaryotic microorganisms such as yeast and fungi. The term also includes cell cultures of any species that can be cultured for the production of a biochemical.

As used herein, the term “non-naturally occurring” when used in reference to a microbial organism described herein is intended to mean that the microbial organism has at least one genetic alteration not normally found in a naturally occurring strain of the referenced species, including wild-type strains of the referenced species. Genetic alterations include, for example, modifications introducing expressible nucleic acids encoding metabolic polypeptides, other nucleic acid additions, nucleic acid deletions and/or other functional disruption of the microbial organism's genetic material. Such modifications include, for example, genetic alterations within coding regions and functional fragments thereof. Additional modifications include, for example, non-coding regulatory regions in which the modifications alter expression of a gene or operon. Exemplary metabolic polypeptides include enzymes or proteins within an acetyl-CoA or bioderived compound pathway described herein.

As use herein, the term “operatively linked” when used in reference to a nucleic acid encoding an engineered aldehyde dehydrogenase refers to connection of a nucleotide sequence encoding an engineered aldehyde dehydrogenase described herein to another nucleotide sequence (e.g., a promoter) is such a way as to allow for the connected nucleotide sequences to function (e.g., express the engineered aldehyde dehydrogenase in the microbial organism).

As used herein, the term “pathway” when used in reference to production of a desired product (e.g., 3-hydroxybutyraldehyde, especially (R)-3-hydroxybutyraldehyde, 4-hydroxybutyraldehyde, 1,3-butanediol, 1,4-butanediol, or an ester or amide of 1,3-butanediol or 1,4-butanediol) refers to one or more polypeptides (e.g., proteins or enzymes) that catalyze the conversion of a substrate compound to a product compound and/or produce a co-substrate for the conversion of a substrate compound to a product compound. Such a product compound can be one of the bioderived compounds described herein, or an intermediate compound that can lead to the bioderived compound upon further conversion by other proteins or enzymes of the metabolic pathway. Accordingly, a metabolic pathway can be comprised of a series of metabolic polypeptides (e.g., two, three, four, five, six, seven, eight, nine, ten or more) that act upon a substrate compound to convert it to a given product compound through a series of intermediate compounds. The metabolic polypeptides of a metabolic pathway can be encoded by an exogenous nucleic acid as described herein or produced naturally by the host microbial organism.

As used herein, the term “recombinant” with respect to a nucleic acid, such as a nucleic acid comprising a gene that encodes a protein or polypeptide (e.g., an engineered aldehyde dehydrogenase described herein), refers to: a nucleic acid that has been artificially supplied to a biological system; a nucleic acid that has been modified within a biological system, or a nucleic acid whose expression or regulation has been manipulated within a biological system. The recombinant nucleic acid can be supplied to the biological system, for example, by introduction of the nucleic acid into genetic material of a microbial organism, such as by integration into a microbial organism chromosome, or as non-chromosomal genetic material such as a plasmid. A recombinant nucleic acid that is introduced into or expressed in a microbial organism may be a nucleic acid that comes from a different organism or species from the microbial organism, or may be a synthetic nucleic acid, or may be a nucleic acid that is also endogenously expressed in the same organism or species as the microbial organism. A recombinant nucleic acid that is also endogenously expressed in the same organism or species as the microbial organism can be considered heterologous if: the sequence of the recombinant nucleic acid is modified relative to the endogenously expressed sequence, the sequence of a regulatory region such as a promoter that controls expression of the nucleic acid is modified relative to the regulatory region of the endogenously expressed sequence, the nucleic acid is expressed in an alternate location in the genome of the microbial organism relative to the endogenously expressed sequence, the nucleic acid is expressed in a different copy number in the microbial organism relative to the endogenously expressed sequence, and/or the nucleic acid is expressed as non-chromosomal genetic material such as a plasmid in the microbial organism.

As used herein, the term “promoter” when used in reference to a nucleic acid encoding an engineered aldehyde dehydrogenase refers to a nucleotide sequence where transcription of a linked open reading frame (e.g., a nucleotide sequence encoding an engineered aldehyde dehydrogenase) by an RNA polymerase begins. A promoter sequence can be located directly upstream or at the 5′ end of the transcription initiation site. RNA polymerase and the necessary transcription factors bind to a promoter sequence and initiate transcription. Promoter sequences define the direction of transcription and indicate which DNA strand will be transcribed, i.e. the sense strand.

As used herein, the term “substantially anaerobic” when used in reference to a culture or growth condition is intended to mean that the amount of dissolved oxygen in a liquid medium is less than about 10% of saturation. The term also is intended to include sealed chambers maintained with an atmosphere of less than about 1% oxygen that include liquid or solid medium.

As used herein, the term “vector” refers to a compound and/or composition that transduces, transforms, or infects a microbial organism, thereby causing the microbial organism to express nucleic acids and/or proteins other than those native to the microbial organism, or in a manner not native to the cell. Vectors can be constructed to include one or more biosynthetic pathway enzyme or protein, such as an engineered FDH described herein, encoded by a nucleotide sequence operably linked to expression control sequences (e.g., promoter) that are functional in the microbial organism (“expression vector”). Expression vectors applicable for use in the microbial organisms described herein include, for example, plasmids, phage vectors, viral vectors, episomes and artificial chromosomes, including vectors and selection sequences or markers operable for stable integration into a host chromosome. Additionally, the expression vectors can include one or more selectable marker genes and appropriate expression control sequences. Selectable marker genes also can be included that, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more recombinant or exogenous encoding nucleic acids are to be co-expressed, both nucleic acids can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The transformation of a recombinant or exogenous nucleic acid encoding an enzyme or protein involved in a metabolic or synthetic pathway can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid or its corresponding gene product (e.g., enzyme or protein). It is understood by those skilled in the art that the recombinant or exogenous nucleic acid is expressed in a sufficient amount to produce the desired product, and it is further understood that expression levels can be optimized to obtain sufficient expression using methods well known in the art and as disclosed herein.

Those skilled in the art will understand that the genetic alterations, including metabolic modifications exemplified herein, are described with reference to a suitable microbial organism such as E. coli and their corresponding metabolic reactions or a suitable source organism for desired genetic material such as genes for a desired metabolic pathway. However, given the complete genome sequencing of a wide variety of organisms and the high level of skill in the area of genomics, those skilled in the art will readily be able to apply the teachings and guidance provided herein to essentially all other organisms. For example, the E. coli metabolic alterations exemplified herein can readily be applied to other species by incorporating the same or analogous encoding nucleic acid from species other than the referenced species. Such genetic alterations include, for example, genetic alterations of species homologs, in general, and in particular, orthologs, paralogs or nonorthologous gene displacements.

An ortholog is a gene or genes that are related by vertical descent and are responsible for substantially the same or identical functions in different organisms. For example, mouse epoxide hydrolase and human epoxide hydrolase can be considered orthologs for the biological function of hydrolysis of epoxides. Genes are related by vertical descent when, for example, they share sequence similarity of sufficient amount to indicate they are homologous, or related by evolution from a common ancestor. Genes can also be considered orthologs if they share three-dimensional structure but not necessarily sequence similarity, of a sufficient amount to indicate that they have evolved from a common ancestor to the extent that the primary sequence similarity is not identifiable. Genes that are orthologous can encode proteins with sequence similarity of about 25% to 100% amino acid sequence identity. Genes encoding proteins sharing an amino acid similarity less that 25% can also be considered to have arisen by vertical descent if their three-dimensional structure also shows similarities. Members of the serine protease family of enzymes, including tissue plasminogen activator and elastase, are considered to have arisen by vertical descent from a common ancestor.

Orthologs include genes or their encoded gene products that through, for example, evolution, have diverged in structure or overall activity. For example, where one species encodes a gene product exhibiting two functions and where such functions have been separated into distinct genes in a second species, the three genes and their corresponding products are considered to be orthologs. For the production of a biochemical product, those skilled in the art will understand that the orthologous gene harboring the metabolic activity to be introduced or disrupted is to be chosen for construction of the non-naturally occurring microbial organism. An example of orthologs exhibiting separable activities is where distinct activities have been separated into distinct gene products between two or more species or within a single species. A specific example is the separation of elastase proteolysis and plasminogen proteolysis, two types of serine protease activity, into distinct molecules as plasminogen activator and elastase. A second example is the separation of mycoplasma 5′-3′ exonuclease and Drosophila DNA polymerase III activity. The DNA polymerase from the first species can be considered an ortholog to either or both of the exonuclease and the polymerase from the second species and vice versa.

In contrast, paralogs are homologs related by, for example, duplication followed by evolutionary divergence and have similar or common, but not identical functions. Paralogs can originate or derive from, for example, the same species or from a different species. For example, microsomal epoxide hydrolase (epoxide hydrolase I) and soluble epoxide hydrolase (epoxide hydrolase II) can be considered paralogs because they represent two distinct enzymes, co-evolved from a common ancestor, that catalyze distinct reactions and have distinct functions in the same species. Paralogs are proteins from the same species with significant sequence similarity to each other suggesting that they are homologous, or related through co-evolution from a common ancestor. Groups of paralogous protein families include HipA homologs, luciferase genes, peptidases, and others.

A nonorthologous gene displacement is a nonorthologous gene from one species that can substitute for a referenced gene function in a different species. Substitution includes, for example, being able to perform substantially the same or a similar function in the species of origin compared to the referenced function in the different species. Although generally, a nonorthologous gene displacement will be identifiable as structurally related to a known gene encoding the referenced function, less structurally related but functionally similar genes and their corresponding gene products nevertheless will still fall within the meaning of the term as it is used herein. Functional similarity requires, for example, at least some structural similarity in the active site or binding region of a nonorthologous gene product compared to a gene encoding the function sought to be substituted. Therefore, a nonorthologous gene includes, for example, a paralog or an unrelated gene.

Therefore, in identifying and constructing the non-naturally occurring microbial organisms described herein having biosynthetic capability for a desired product, those skilled in the art will understand with applying the teaching and guidance provided herein to a particular species that the identification of metabolic modifications can include identification and inclusion or inactivation of orthologs. To the extent that paralogs and/or nonorthologous gene displacements are present in the referenced microbial organism that encode an enzyme catalyzing a similar or substantially similar metabolic reaction, those skilled in the art also can utilize these evolutionally related genes. Similarly, for a gene disruption, evolutionally related genes can also be disrupted or deleted in a microbial organism to reduce or eliminate functional redundancy of enzymatic activities targeted for disruption.

Orthologs, paralogs and nonorthologous gene displacements can be determined by methods well known to those skilled in the art. For example, inspection of nucleic acid or amino acid sequences for two polypeptides will reveal sequence identity and similarities between the compared sequences. Based on such similarities, one skilled in the art can determine if the similarity is sufficiently high to indicate the proteins are related through evolution from a common ancestor. Algorithms well known to those skilled in the art, such as Align, BLAST, Clustal W and others compare and determine a raw sequence similarity or identity, and also determine the presence or significance of gaps in the sequence which can be assigned a weight or score. Such algorithms also are known in the art and are similarly applicable for determining nucleotide sequence similarity or identity. Parameters for sufficient similarity to determine relatedness are computed based on well known methods for calculating statistical similarity, or the chance of finding a similar match in a random polypeptide, and the significance of the match determined. A computer comparison of two or more sequences can, if desired, also be optimized visually by those skilled in the art. Related gene products or proteins can be expected to have a high similarity, for example, 25% to 100% sequence identity. Proteins that are unrelated can have an identity which is essentially the same as would be expected to occur by chance, if a database of sufficient size is scanned (about 5%). Sequences between 5% and 24% may or may not represent sufficient homology to conclude that the compared sequences are related. Additional statistical analysis to determine the significance of such matches given the size of the data set can be carried out to determine the relevance of these sequences.

Exemplary parameters for determining relatedness of two or more sequences using the BLAST algorithm, for example, can be as set forth below. Briefly, amino acid sequence alignments can be performed using BLASTP version 2.0.8 (Jan. 5, 1999) and the following parameters: Matrix: 0 BLOSUM62; gap open: 11; gap extension: 1; x_dropoff: 50; expect: 10.0; wordsize: 3; filter: on. Nucleotide sequence alignments can be performed using BLASTN version 2.0.6 (Sep. 16, 1998) and the following parameters: Match: 1; mismatch: −2; gap open: 5; gap extension: 2; x_dropoff: 50; expect: 10.0; wordsize: 11; filter: off. Those skilled in the art will know what modifications can be made to the above parameters to either increase or decrease the stringency of the comparison, for example, and determine the relatedness of two or more sequences.

An engineered aldehyde dehydrogenase described herein converts an acyl-CoA to its corresponding aldehyde. Such an enzyme can also be referred to as an oxidoreductase that converts an acyl-CoA to its corresponding aldehyde. Such an engineered aldehyde dehydrogenase described herein can be classified as a reaction 1.2.1.b, oxidoreductase (acyl-CoA to aldehyde), where the first three digits correspond to the first three Enzyme Commission number digits which denote the general type of transformation independent of substrate specificity. Exemplary enzymatic conversions of an engineered aldehyde dehydrogenase provided herein include, but are not limited to, the conversion of 3-hydroxybutyryl-CoA to 3-hydroxybutyraldehyde and the conversion of 4-hydroxybutyryl-CoA to 4-hydroxybutyraldehyde. An aldehyde dehydrogenase described herein can be used to produce desired products, such as 3-hydroxybutyraldehyde, 1,3-BDO, 4-hydroxybutyraldehyde, 1,4-BDO, or other desired products such as a downstream product, including an ester or amide thereof, in a cell, such as a microbial organism, containing a suitable metabolic pathway, or in vitro. For example, 1,3-BDO can be reacted with an acid, either in vivo or in vitro, to convert to an ester using, for example, a lipase. Such esters can have nutraceutical, medical and food uses, and are advantaged when R-form of 1,3-BDO is used since that is the form (compared to S-form or the racemic mixture that is made from petroleum or from ethanol by the acetaldehyde chemical synthesis route) best utilized by both animals and humans as an energy source (e.g., a ketone ester, such as (R)-3-hydroxybutyl-R-1,3-butanediol monoester (which has Generally Recognized As Safe (GRAS) approval in the United States) and (R)-3-hydroxybutyrate glycerol monoester or diester). The ketone esters can be delivered orally, and the ester releases R-1,3-BDO that is used by the body (see, for example, WO 2013/150153). Thus, an aldehyde dehydrogenase described herein is particularly useful to provide an improved enzymatic route and microorganism to provide an improved composition of 1,3-BDO, namely R-1,3-BDO, highly enriched or essentially enantiomerically pure, and further having improved purity qualities with respect to by-products.

In some embodiments, provided herein is an engineered aldehyde dehydrogenase that is a variant of a wild-type aldehyde dehydrogenase (SEQ ID NO: 1) or a parent aldehyde dehydrogenase (SEQ ID NO: 3). Such an engineered aldehyde dehydrogenase includes one or more alterations at a position described in TABLE 2, and has higher catalytic activity relative to the wild-type aldehyde dehydrogenase (SEQ ID NO: 1) or the parent aldehyde dehydrogenase (SEQ ID NO: 3) as described herein. In some embodiments, an engineered aldehyde dehydrogenase provided herein is capable of catalyzing the conversion of: 1) 3-hydroxybutyryl-CoA to 3-hydroxybutyraldehyde; 2) (R)-3-hydroxybutyryl-CoA to (R)-3-hydroxybutyraldehyde, and/or 3) 4-hydroxybutyryl-CoA to 4-hydroxybutyraldehyde. Accordingly, in some embodiments, an engineered aldehyde dehydrogenase provided herein is capable of catalyzing the conversion of 3-hydroxybutyryl-CoA to 3-hydroxybutyraldehyde. In some embodiments, an engineered aldehyde dehydrogenase provided herein is capable of catalyzing the conversion of (R)-3-hydroxybutyryl-CoA to (R)-3-hydroxybutyraldehyde. In some embodiments, an engineered aldehyde dehydrogenase provided herein is capable of catalyzing the conversion of 4-hydroxybutyryl-CoA to 4-hydroxybutyraldehyde.

In some embodiments, an engineered aldehyde dehydrogenase as described herein has higher catalytic activity in the conversion of select substrates over other substrates. For example, in some embodiments, an engineered aldehyde dehydrogenase as described herein has: 1) higher specificity for conversion of 3-hydroxybutyryl-CoA to 3-hydroxybutyraldehyde over conversion of acetyl-CoA to acetaldehyde; 2) higher specificity for conversion of (R)-3-hydroxybutyryl-CoA to (R)-3-hydroxybutyraldehyde over conversion of (S)-3-hydroxybutyryl-CoA to (S)-3-hydroxybutyraldehyde; and/or 3) higher specificity for conversion of 4-hydroxybutyryl-CoA to 4-hydroxybutyraldehyde over conversion of acetyl-CoA to acetaldehyde. Accordingly, in some embodiments, an engineered aldehyde dehydrogenase provided herein has higher specificity for conversion of 3-hydroxybutyryl-CoA to 3-hydroxybutyraldehyde over conversion of acetyl-CoA to acetaldehyde. In some embodiments, an engineered aldehyde dehydrogenase provided herein has higher specificity for conversion of (R)-3-hydroxybutyryl-CoA to (R)-3-hydroxybutyraldehyde over conversion of (S)-3-hydroxybutyryl-CoA to (S)-3-hydroxybutyraldehyde. In some embodiments, an engineered aldehyde dehydrogenase provided herein has higher specificity for conversion of 4-hydroxybutyryl-CoA to 4-hydroxybutyraldehyde over conversion of acetyl-CoA to acetaldehyde.

Exemplary enzymatic reactions catalyzed by an engineered aldehyde dehydrogenase described herein is represented by:

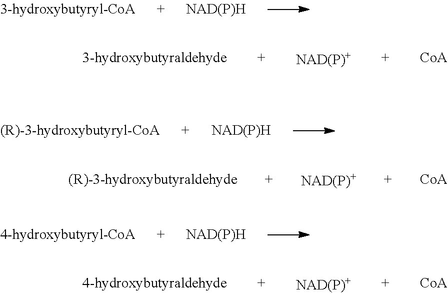

In some embodiments, provided herein is an engineered aldehyde dehydrogenase comprising a variant of amino acid sequence SEQ ID NO: 3, wherein the engineered aldehyde dehydrogenase comprises one or more alterations at a position described in TABLE 2. In some embodiments, such an engineered aldehyde dehydrogenase is capable of catalyzing the conversion of: 1) 3-hydroxybutyryl-CoA to 3-hydroxybutyraldehyde; 2) (R)-3-hydroxybutyryl-CoA to (R)-3-hydroxybutyraldehyde, and/or 3) 4-hydroxybutyryl-CoA to 4-hydroxybutyraldehyde. Accordingly, in some embodiments, such an engineered aldehyde dehydrogenase comprising a variant of amino acid sequence SEQ ID NO: 3 is capable of catalyzing the conversion of 3-hydroxybutyryl-CoA to 3-hydroxybutyraldehyde. In some embodiments, such an engineered aldehyde dehydrogenase comprising a variant of amino acid sequence SEQ ID NO: 3 is capable of catalyzing the conversion of (R)-3-hydroxybutyryl-CoA to (R)-3-hydroxybutyraldehyde. In some embodiments, such an engineered aldehyde dehydrogenase comprising a variant of amino acid sequence SEQ ID NO: 3 is capable of catalyzing the conversion of 4-hydroxybutyryl-CoA to 4-hydroxybutyraldehyde.

It is understood that the engineered aldehyde dehydrogenases as described herein can carry out a similar enzymatic reaction as the wild-type aldehyde dehydrogenase (SEQ ID NO: 1) or the parent aldehyde dehydrogenase (SEQ ID NO: 3) as discussed above. It is further understood that the variants of the aldehyde dehydrogenase enzyme can include alterations that provide a beneficial characteristic to the engineered aldehyde dehydrogenase, including but not limited to, increased activity (e.g., ability to catalyze a reaction described herein and/or selectivity for a substrate, such as 3-hydroxybutyryl-CoA, (R)-3-hydroxybutyryl-CoA, or 4-hydroxybutyryl-CoA) as described herein (see, e.g., Examples II and III). In some embodiments, the engineered aldehyde dehydrogenase can exhibit an activity that is at least the same or higher than the wild-type aldehyde dehydrogenase (SEQ ID NO: 1) or the parent aldehyde dehydrogenase (SEQ ID NO: 3), that is, it has activity that is the same or higher than an aldehyde dehydrogenase without the variant at the same amino acid position(s). In some embodiments, the engineered aldehyde dehydrogenase can exhibit two or more activities (e.g., ability to catalyze a reaction described herein and selectivity for a substrate, such as 3-hydroxybutyryl-CoA, (R)-3-hydroxybutyryl-CoA, or 4-hydroxybutyryl-CoA) that are at least the same or higher than the wild-type aldehyde dehydrogenase (SEQ ID NO: 1) or the parent aldehyde dehydrogenase (SEQ ID NO: 3), that is, it has two or more activities that are the same or higher than an aldehyde dehydrogenase without the variant at the same amino acid position(s). For example, the engineered aldehyde dehydrogenases provided here can have one or more activity that is at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, or at least 200% higher over a wild-type or parent aldehyde dehydrogenase (see, e.g., Examples II and III). In some embodiments, an engineered aldehyde dehydrogenase provided herein has an activity that is at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, or at least 200% higher than the activity of an aldehyde dehydrogenase consisting of the amino acid sequence of SEQ ID NO: 1. In some embodiments, an engineered aldehyde dehydrogenase provided herein has an activity that is at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, or at least 200% higher than the activity of an aldehyde dehydrogenase consisting of the amino acid sequence of SEQ ID NO: 3. In some embodiments, an engineered aldehyde dehydrogenase provided herein has an activity that is at least 10% higher. In some embodiments, an engineered aldehyde dehydrogenase provided herein has an activity that is at least 20% higher. In some embodiments, an engineered aldehyde dehydrogenase provided herein has an activity that is at least 30% higher. In some embodiments, an engineered aldehyde dehydrogenase provided herein has an activity that is at least 40% higher. In some embodiments, an engineered aldehyde dehydrogenase provided herein has an activity that is at least 50% higher. In some embodiments, an engineered aldehyde dehydrogenase provided herein has an activity that is at least 60% higher. In some embodiments, an engineered aldehyde dehydrogenase provided herein has an activity that is at least 70% higher. In some embodiments, an engineered aldehyde dehydrogenase provided herein has an activity that is at least 80% higher. In some embodiments, an engineered aldehyde dehydrogenase provided herein has an activity that is at least 90% higher. In some embodiments, an engineered aldehyde dehydrogenase provided herein has an activity that is at least 100% higher. In some embodiments, an engineered aldehyde dehydrogenase provided herein has an activity that is at least 110% higher. In some embodiments, an engineered aldehyde dehydrogenase provided herein has an activity that is at least 120% higher. In some embodiments, an engineered aldehyde dehydrogenase provided herein has an activity that is at least 130% higher. In some embodiments, an engineered aldehyde dehydrogenase provided herein has an activity that is at least 140% higher. In some embodiments, an engineered aldehyde dehydrogenase provided herein has an activity that is at least 150% higher. In some embodiments, an engineered aldehyde dehydrogenase provided herein has an activity that is at least 160% higher. In some embodiments, an engineered aldehyde dehydrogenase provided herein has an activity that is at least 170% higher. In some embodiments, an engineered aldehyde dehydrogenase provided herein has an activity that is at least 180% higher. In some embodiments, an engineered aldehyde dehydrogenase provided herein has an activity that is at least 190% higher. In some embodiments, an engineered aldehyde dehydrogenase provided herein has an activity that is at least 200% higher. It is understood that activity refers to the ability of an engineered aldehyde dehydrogenase described herein to convert a substrate to a product relative to a wild-type aldehyde dehydrogenase (SEQ ID NO: 1) or a parent aldehyde dehydrogenase (SEQ ID NO: 3) under the same assay conditions, such as those described herein (see, e.g., Examples II and III).

In some embodiments, the activity of an aldehyde dehydrogenase described herein is measured as the catalytic constant (k_{cat}) value or turnover number. In some embodiments, the k_{cat} is at least 0.1 s⁻¹, at least 0.2 s⁻¹, at least 0.3 s⁻¹, at least 0.4 s⁻¹, at least 0.5 s⁻¹, at least 0.6 s⁻¹, at least 0.7 s⁻¹, at least 0.8 s⁻¹, at least 0.9 s⁻¹, at least 1 s⁻¹, at least 2 s⁻¹, at least 3 s⁻¹, at least 4 s⁻¹, at least 5 s⁻¹, at least 6 s⁻¹, at least 7 s⁻¹, at least 8 s⁻¹, at least 9 s⁻¹, at least 10 s⁻¹, at least 11 s⁻¹, at least 12 s⁻¹, at least 13 s⁻¹, at least 14 s⁻¹, at least 15 s⁻¹, at least 16 s⁻¹, at least 17 s⁻¹, at least 18 s⁻¹, at least 19 s⁻¹, at least 20 s⁻¹, at least 21 s⁻¹, at least 22 s⁻¹, at least 23 s⁻¹, at least 24 s⁻¹, at least 25 s⁻¹, at least 26 s⁻¹, at least 27 s⁻¹, at least 28 s⁻¹, at least 29 s⁻¹, at least 30 s⁻¹, at least 31 s⁻¹, at least 32 s⁻¹, at least 33 s⁻¹, at least 34 s⁻¹, at least 35 s⁻¹, at least 36 s⁻¹, at least 37 s⁻¹, at least 38 s⁻¹, at least 39 s⁻¹, at least 40 s⁻¹, at least 41 s⁻¹, at least 42 s⁻¹, at least 43 s⁻¹, at least 44 s⁻¹, at least 45 s⁻¹, at least 46 s⁻¹, at least 47 s⁻¹, at least 48 s⁻¹, at least 49 s⁻¹, at least 50 s⁻¹, at least 51 s⁻¹, at least 52 s⁻¹, at least 53 s⁻¹, at least 54 s⁻¹, at least 55 s⁻¹, at least 56 s⁻¹, at least 57 s⁻¹, at least 58 s⁻¹, at least 59 s⁻¹, at least 60 s⁻¹, at least 61 s⁻¹, at least 62 s⁻¹, at least 63 s⁻¹, at least 64 s⁻¹, at least 65 s⁻¹, at least 66 s⁻¹, at least 67 s⁻¹, at least 68 s⁻¹, at least 69 s⁻¹, at least 70 s⁻¹, at least 71 s⁻¹, at least 72 s⁻¹, at least 73 s⁻¹, at least 74 s⁻¹, at least 75 s⁻¹, at least 76 s⁻¹, at least 77 s⁻¹, at least 78 s⁻¹, at least 79 s⁻¹, at least 80 s⁻¹, at least 81 s⁻¹, at least 82 s⁻¹, at least 83 s⁻¹, at least 84 s⁻¹, at least 85 s⁻¹, at least 86 s⁻¹, at least 87 s⁻¹, at least 88 s⁻¹, at least 89 s⁻¹, at least 90 s⁻¹, at least 91 s⁻¹, at least 92 s⁻¹, at least 93 s⁻¹, at least 94 s⁻¹, at least 95 s⁻¹, at least 96 s⁻¹, at least 97 s⁻¹, at least 98 s⁻¹, at least 99 s⁻¹, at least 100 s⁻¹, at least 500 s⁻¹, or at least 1000 s⁻¹. In some embodiments, the Keat is between 1 s⁻¹ and 100 s⁻¹, between 5 s⁻¹ and 50 s⁻¹, or between 10 s⁻¹ and 50 s⁻¹.

In some embodiments, the activity of an aldehyde dehydrogenase described herein is measured as the Michaelis constant (Kₘ). In some embodiments, the Kₘ is less than 0.1 μM, less than 0.2 μM, less than 0.3 μM, less than 0.4 μM, less than 0.5 μM, less than 0.6 μM, less than 0.7 μM, less than 0.8 μM, less than 0.9 μM, less than 1 μM, less than 2 μM, less than 3 μM, less than 4 μM, less than 5 μM, less than 6 μM, less than 7 μM, less than 8 μM, less than 9 μM, less than 10 μM, less than 11 μM, less than 12 μM, less than 13 μM, less than 14 μM, less than 15 μM, less than 16 μM, less than 17 μM, less than 18 μM, less than 19 μM, less than 20 μM, less than 21 μM, less than 22 μM, less than 23 μM, less than 24 μM, less than 25 μM, less than 26 μM, less than 27 μM, less than 28 μM, less than 29 μM, less than 30 μM, less than 31 μM, less than 32 μM, less than 33 μM, less than 34 μM, less than 35 μM, less than 36 μM, less than 37 μM, less than 38 μM, less than 39 μM, less than 40 μM, less than 41 μM, less than 42 μM, less than 43 μM, less than 44 μM, less than 45 μM, less than 46 μM, less than 47 μM, less than 48 μM, less than 49 μM, less than 50 μM, less than 51 μM, less than 52 μM, less than 53 μM, less than 54 μM, less than 55 μM, less than 56 μM, less than 57 μM, less than 58 μM, less than 59 μM, less than 60 μM, less than 61 μM, less than 62 μM, less than 63 μM, less than 64 μM, less than 65 μM, less than 66 μM, less than 67 μM, less than 68 μM, less than 69 μM, less than 70 μM, less than 71 μM, less than 72 μM, less than 73 μM, less than 74 μM, less than 75 μM, less than 76 μM, less than 77 μM, less than 78 μM, less than 79 μM, less than 80 μM, less than 81 μM, less than 82 μM, less than 83 μM, less than 84 μM, less than 85 μM, less than 86 μM, less than 87 μM, less than 88 μM, less than 89 μM, less than 90 μM, less than 91 μM, less than 92 μM, less than 93 μM, less than 94 μM, less than 95 μM, less than 96 μM, less than 97 μM, less than 98 μM, less than 99 μM, less than 100 μM, less than 200 μM, less than 300 μM, less than 400 μM, less than 500 μM, less than 600 μM, less than 700 μM, less than 800 μM, less than 900 μM, less than 1000 μM, less than 1100 μM, less than 1200 μM, less than 1300 μM, less than 1400 μM, less than 1500 μM, less than 1600 μM, less than 1700 μM, less than 1800 μM, less than 1900 μM, or less than 2000 μM. In some embodiments, the Kₘ is between 0.1 μM and 2000 μM, between 1 μM and 1000 μM, between 1 μM and 100 μM, between 0.1 μM and 1000 μM, between 100 μM and 2000 μM, between 100 μM and 1000 μM, between 1000 μM and 2000 μM, between 500 μM and 1500 μM, between 500 μM and 1500 μM, or between 0.1 μM and 1000 μM.

In some embodiments, the activity of an aldehyde dehydrogenase described herein is measured as the catalytic efficiency (k_{cat}/kₘ). In some embodiments, the catalytic efficiency is measured in units of s⁻¹ mM⁻¹. In some embodiments, the catalytic efficiency is greater than 0.1, greater than 0.2, greater than 0.3, greater than 0.4, greater than 0.5, greater than 0.6, greater than 0.7, greater than 0.8, greater than 0.9, greater than 1, greater than 2, greater than 3, greater than 4, greater than 5, greater than 6, greater than 7, greater than 8, greater than 9, greater than 10, greater than 11, greater than 12, greater than 13, greater than 14, greater than 15, greater than 16, greater than 17, greater than 18, greater than 19, greater than 20, greater than 21, greater than 22, greater than 23, greater than 24, greater than 25, greater than 26, greater than 27, greater than 28, greater than 29, greater than 30, greater than 31, greater than 32, greater than 33, greater than 34, greater than 35, greater than 36, greater than 37, greater than 38, greater than 39, greater than 40, greater than 41, greater than 42, greater than 43, greater than 44, greater than 45, greater than 46, greater than 47, greater than 48, greater than 49, greater than 50, greater than 51, greater than 52, greater than 53, greater than 54, greater than 55, greater than 56, greater than 57, greater than 58, greater than 59, greater than 60, greater than 61, greater than 62, greater than 63, greater than 64, greater than 65, greater than 66, greater than 67, greater than 68, greater than 69, greater than 70, greater than 71, greater than 72, greater than 73, greater than 74, greater than 75, greater than 76, greater than 77, greater than 78, greater than 79, greater than 80, greater than 81, greater than 82, greater than 83, greater than 84, greater than 85, greater than 86, greater than 87, greater than 88, greater than 89, greater than 90, greater than 91, greater than 92, greater than 93, greater than 94, greater than 95, greater than 96, greater than 97, greater than 98, greater than 99, greater than 100, greater than 500, greater than 1000 s⁻¹ mM⁻¹. In some embodiments, the catalytic efficiency (k_{cat}/kₘ) is between 1 and 30 s⁻¹ mM⁻¹, between 5 and 30 s⁻¹ mM⁻¹, between 1 and 10 s⁻¹ mM⁻¹, between 10 and 30 s⁻¹ mM⁻¹, or between 20 and 30 s⁻¹ mM⁻¹.

In some embodiments, an engineered aldehyde dehydrogenase provided herein is a variant of a reference polypeptide, wherein the reference polypeptide has an amino acid sequence of SEQ ID NO: 3, and the engineered aldehyde dehydrogenase has one or more alterations at a position described in TABLE 2 relative to SEQ ID NO: 3. Accordingly, in some embodiments, an engineered aldehyde dehydrogenase provided herein includes one or more amino acid alterations at a residue corresponding to position 142, 243, 277, 401, 435, or 442, or a combination thereof, in SEQ ID NO: 3. In some embodiments, an engineered aldehyde dehydrogenase provided herein includes one or more amino acid alterations at a residue corresponding to position 435 or 442, or a combination thereof, in SEQ ID NO: 3.

In some embodiments, an engineered aldehyde dehydrogenase provided herein includes one or more alterations at a position described in TABLE 2, wherein the one or more amino acid alterations are conservative amino acid substitutions. In some embodiments, an engineered aldehyde dehydrogenase provided herein includes one or more conservative amino acid substitutions relative to an alteration described in TABLE 2. As a non-limiting example, a conservative amino acid substitution relative to the A277C substitution in SEQ ID NO: 3 may include substitution of A277 for another non-polar (hydrophobic) (e.g., Val (V), Leu (L), Ile (I), Pro (P), Phe (F), Met (M), Trp (W), Gly (G), or Tyr (Y)). In some embodiments, an engineered aldehyde dehydrogenase provided herein includes one or more alterations at a position described in TABLE 2, wherein the one or more amino acid alterations are non-conservative amino acid substitutions. In some embodiments, an engineered aldehyde dehydrogenase provided herein includes a conservative amino acid substitution and/or non-conservative amino acid substitution in 1 to 7 amino acid positions as set forth in TABLE 2.

In some embodiments, an engineered aldehyde dehydrogenase provided herein can further include a conservative amino acid substitution in from 1 to 50 amino acid positions, or alternatively from 2 to 50 amino acid positions, or alternatively from 3 to 50 amino acid positions, or alternatively from 4 to 50 amino acid positions, or alternatively from 5 to 50 amino acid positions, or alternatively from 6 to 50 amino acid positions, or alternatively from 7 to 50 amino acid positions, or alternatively from 8 to 50 amino acid positions, or alternatively from 9 to 50 amino acid positions, or alternatively from 10 to 50 amino acid positions, or alternatively from 15 to 50 amino acid positions, or alternatively from 20 to 50 amino acid positions, or alternatively from 30 to 50 amino acid positions, or alternatively from 40 to 50 amino acid positions, or alternatively from 45 to 50 amino acid positions, or any integer therein, wherein the positions are other than the variant amino acid positions set forth in TABLE 2. In some aspects, such a conservative amino acid sequence is a chemically conservative or an evolutionary conservative amino acid substitution. Methods of identifying conservative amino acids are well known to one of skill in the art, any one of which can be used to generate the isolated engineered aldehyde dehydrogenases described herein.

An engineered aldehyde dehydrogenase provided herein may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, or 250 alterations relative to a wild-type aldehyde dehydrogenase (SEQ ID NO: 1) or parent aldehyde dehydrogenase (SEQ ID NO: 3). An engineered aldehyde dehydrogenase provided herein may comprise at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 21, at most 22, at most 23, at most 24, at most 25, at most 26, at most 27, at most 28, at most 29, at most 30, at most 31, at most 32, at most 33, at most 34, at most 35, at most 36, at most 37, at most 38, at most 39, at most 40, at most 41, at most 42, at most 43, at most 44, at most 45, at most 46, at most 47, at most 48, at most 49, at most 50, at most 51, at most 52, at most 53, at most 54, at most 55, at most 56, at most 57, at most 58, at most 59, at most 60, at most 61, at most 62, at most 63, at most 64, at most 65, at most 66, at most 67, at most 68, at most 69, at most 70, at most 71, at most 72, at most 73, at most 74, at most 75, at most 76, at most 77, at most 78, at most 79, at most 80, at most 81, at most 82, at most 83, at most 84, at most 85, at most 86, at most 87, at most 88, at most 89, at most 90, at most 91, at most 92, at most 93, at most 94, at most 95, at most 96, at most 97, at most 98, at most 99, at most 100, at most 101, at most 102, at most 103, at most 104, at most 105, at most 106, at most 107, at most 108, at most 109, at most 110, at most 111, at most 112, at most 113, at most 114, at most 115, at most 116, at most 117, at most 118, at most 119, at most 120, at most 121, at most 122, at most 123, at most 124, at most 125, at most 126, at most 127, at most 128, at most 129, at most 130, at most 131, at most 132, at most 133, at most 134, at most 135, at most 136, at most 137, at most 138, at most 139, at most 140, at most 141, at most 142, at most 143, at most 144, at most 145, at most 146, at most 147, at most 148, at most 149, at most 150, at most 151, at most 152, at most 153, at most 154, at most 155, at most 156, at most 157, at most 158, at most 159, at most 160, at most 161, at most 162, at most 163, at most 164, at most 165, at most 166, at most 167, at most 168, at most 169, at most 170, at most 171, at most 172, at most 173, at most 174, at most 175, at most 176, at most 177, at most 178, at most 179, at most 180, at most 181, at most 182, at most 183, at most 184, at most 185, at most 186, at most 187, at most 188, at most 189, at most 190, at most 191, at most 192, at most 193, at most 194, at most 195, at most 196, at most 197, at most 198, at most 199, at most 200, at most 201, at most 202, at most 203, at most 204, at most 205, at most 206, at most 207, at most 208, at most 209, at most 210, at most 211, at most 212, at most 213, at most 214, at most 215, at most 216, at most 217, at most 218, at most 219, at most 220, at most 221, at most 222, at most 223, at most 224, at most 225, at most 226, at most 227, at most 228, at most 229, at most 230, at most 231, at most 232, at most 233, at most 234, at most 235, at most 236, at most 237, at most 238, at most 239, at most 240, at most 241, at most 242, at most 243, at most 244, at most 245, at most 246, at most 247, at most 248, at most 249, or at most 250 alterations relative to a wild-type aldehyde dehydrogenase (SEQ ID NO: 1) or a parent aldehyde dehydrogenase (SEQ ID NO: 3). The one or more alterations may be located at one or more positions corresponding to the one or more positions described in TABLE 2. The one or more alterations may be located at one or more positions corresponding to one or more positions in SEQ ID NO: 3. As used herein, the phrase “a residue corresponding to position X in SEQ ID NO: Y” refers to a residue at a corresponding position following an alignment of two sequences. For example, the residue in SEQ ID NO: 1 corresponding to position 370 in SEQ ID NO: 3 is the residue at position 370 in SEQ ID NO: 1. In some embodiments, a reference sequence is an aldehyde dehydrogenase that is not SEQ ID NO: 1 or 3.

An engineered aldehyde dehydrogenase provided herein can include any combination of the alterations set forth in TABLE 2. One alteration alone, or in combination, can produce an engineered aldehyde dehydrogenase that retains or improves the activity as described herein relative to a reference polypeptide, for example, the wild-type aldehyde dehydrogenase (SEQ ID NO: 1) or the parent aldehyde dehydrogenase (SEQ ID NO: 3). In some embodiments, an engineered aldehyde dehydrogenase provided herein includes at least 2, 3, 4, 5, 6, or 7 alterations as set forth in TABLE 2, including up to an alteration at all of the positions identified in TABLE 2. In some embodiments, an engineered aldehyde dehydrogenase provided herein includes at least 2 alterations as set forth in TABLE 2. In some embodiments, an engineered aldehyde dehydrogenase provided herein includes at least 3 alterations as set forth in TABLE 2. In some embodiments, an engineered aldehyde dehydrogenase provided herein includes at least 4 alterations as set forth in TABLE 2. In some embodiments, an engineered aldehyde dehydrogenase provided herein includes at least 5 alterations as set forth in TABLE 2. In some embodiments, an engineered aldehyde dehydrogenase provided herein includes at least 6 alterations as set forth in TABLE 2. In some embodiments, an engineered aldehyde dehydrogenase provided herein includes at least 7 alterations as set forth in TABLE 2.

In some embodiments, the one or more amino acid alterations of the engineered aldehyde dehydrogenase is an alteration described in TABLE 2. For example, in some embodiments, the one or more amino acid alternations result in an engineered aldehyde dehydrogenase having: a) I or V at a residue corresponding to position 142 in SEQ ID NO: 3; b) C at a residue corresponding to position 146 in SEQ ID NO: 3; c) E at a residue corresponding to position 243 in SEQ ID NO: 3; d) C at a residue corresponding to position 277 in SEQ ID NO: 3; e) F at a residue corresponding to position 401 in SEQ ID NO: 3; f) H, M, Q, or R at a residue corresponding to position 435 in SEQ ID NO: 3; and/or g) F at a residue corresponding to position 442 in SEQ ID NO: 3.

In some embodiments, the one or more amino acid alterations of the engineered aldehyde dehydrogenase is an alteration described in TABLE 3, which results in the engineered aldehyde dehydrogenase producing 1,3-BDO when the engineered aldehyde dehydrogenase is expressed in an organism having a pathway for production of 1,3-BDO and assayed under conditions as described in Example III. For example, in some embodiments, the one or more amino acid alternations result in an engineered aldehyde dehydrogenase having: a) M or R at a residue corresponding to position 435 in SEQ ID NO: 3; and/or b) F at a residue corresponding to position 442 in SEQ ID NO: 3.

In some embodiments, an engineered aldehyde dehydrogenase described herein has: I at a residue corresponding to position 142 in SEQ ID NO: 3.

In some embodiments, an engineered aldehyde dehydrogenase described herein has: V at a residue corresponding to position 142 in SEQ ID NO: 3.

In some embodiments, an engineered aldehyde dehydrogenase described herein has: C at a residue corresponding to position 146 in SEQ ID NO: 3.

In some embodiments, an engineered aldehyde dehydrogenase described herein has: E at a residue corresponding to position 243 in SEQ ID NO: 3.

In some embodiments, an engineered aldehyde dehydrogenase described herein has: C at a residue corresponding to position 277 in SEQ ID NO: 3.

In some embodiments, an engineered aldehyde dehydrogenase described herein has: F at a residue corresponding to position 401 in SEQ ID NO: 3.

In some embodiments, an engineered aldehyde dehydrogenase described herein has: H at a residue corresponding to position 435 in SEQ ID NO: 3.

In some embodiments, an engineered aldehyde dehydrogenase described herein has: M at a residue corresponding to position 435 in SEQ ID NO: 3.

In some embodiments, an engineered aldehyde dehydrogenase described herein has: Q at a residue corresponding to position 435 in SEQ ID NO: 3.

In some embodiments, an engineered aldehyde dehydrogenase described herein has: R at a residue corresponding to position 435 in SEQ ID NO: 33.

In some embodiments, an engineered aldehyde dehydrogenase described herein has: F at a residue corresponding to position 442 in SEQ ID NO: 3.

In some embodiments, an engineered aldehyde dehydrogenase provided herein has an amino acid sequence that is a variant of SEQ ID NO: 3 that includes one or more alterations as described in TABLE 2, wherein the portion, other than the one or more alterations described in TABLE 2, of the engineered aldehyde dehydrogenase has at least 65%, 70%, 75%, 80%, 85%, 90%, 95%, 98% or 99% sequence identity, or is identical, to an amino acid sequence referenced as SEQ ID NO: 3. Accordingly, in some embodiments, an engineered aldehyde dehydrogenase provided herein has an amino acid sequence that includes one or more alterations as described in TABLE 2 and the portion, other than the alteration described in TABLE 2, of the engineered aldehyde dehydrogenase has at least 65% identical to SEQ ID NO: 3. In some embodiments, an engineered aldehyde dehydrogenase provided herein has an amino acid sequence that includes one or more alterations as described in TABLE 2 and the portion, other than the alteration described in TABLE 2 or the combination of alterations described in, of the engineered aldehyde dehydrogenase has at least 70% identical to SEQ ID NO: 3. In some embodiments, an engineered aldehyde dehydrogenase provided herein has an amino acid sequence that includes one or more alterations as described in TABLE 2 and the portion, other than the alteration described in TABLE 2, of the engineered aldehyde dehydrogenase has at least 75% identical to SEQ ID NO: 3. In some embodiments, an engineered aldehyde dehydrogenase provided herein has an amino acid sequence that includes one or more alterations as described in TABLE 2 and the portion, other than the alteration described in TABLE 2, of the engineered aldehyde dehydrogenase has at least 80% identical to SEQ ID NO: 3. In some embodiments, an engineered aldehyde dehydrogenase provided herein has an amino acid sequence that includes one or more alterations as described in TABLE 2 and the portion, other than the alteration described in TABLE 2, of the engineered aldehyde dehydrogenase has at least 85% identical to SEQ ID NO: 3. In some embodiments, an engineered aldehyde dehydrogenase provided herein has an amino acid sequence that includes one or more alterations as described in TABLE 2 and the portion, other than the alteration described in TABLE 2, of the engineered aldehyde dehydrogenase has at least 90% identical to SEQ ID NO: 3. In some embodiments, an engineered aldehyde dehydrogenase provided herein has an amino acid sequence that includes one or more alterations as described in TABLE 2 and the portion, other than the alteration described in TABLE 2, of the engineered aldehyde dehydrogenase has at least 95% identical to SEQ ID NO: 3. In some embodiments, an engineered aldehyde dehydrogenase provided herein has an amino acid sequence that includes one or more alterations as described in TABLE 2 and the portion, other than the alteration described in TABLE 2, of the engineered aldehyde dehydrogenase has at least 98% identical to SEQ ID NO: 3. In some embodiments, an engineered aldehyde dehydrogenase provided herein has an amino acid sequence that includes one or more alterations as described in TABLE 2 and the portion, other than the alteration described in TABLE 2, of the engineered aldehyde dehydrogenase has at least 99% identical to SEQ ID NO: 3. In some embodiments, an engineered aldehyde dehydrogenase provided herein has an amino acid sequence that includes one or more alterations as described in TABLE 2 and the portion, other than the alteration described in TABLE 2, of the engineered aldehyde dehydrogenase is identical to SEQ ID NO: 3.

Sequence identity, homology or similarity refers to sequence similarity between two polypeptides or between two nucleic acid molecules. Identity can be determined by comparing a position in each sequence which may be aligned for purposes of comparison. When a position in the compared sequence is occupied by the same base or amino acid, then the molecules are identical at that position. A degree of homology between sequences is a function of the number of matching or homologous positions shared by the sequences. A polypeptide or polypeptide region (or a polynucleotide or polynucleotide region) has a certain percentage (for example, 65%, 70%, 75%, 80%, 85%, 90%, 95%, 98% or 99%) of “sequence identity” to another sequence means that, when aligned, that percentage of amino acids (or nucleotide bases) are the same in comparing the two sequences. The alignment of two sequences to determine their percent sequence identity can be done using software programs known in the art, such as, for example, those described in Ausubel et al., Current Protocols in Molecular Biology, John Wiley and Sons, Baltimore, MD (1999). Preferably, default parameters are used for the alignment. One alignment program well known in the art that can be used is BLAST set to default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by =HIGH SCORE; Databases=non-redundant, GenBank+EMBL+DDBJ+PDB+GenBank CDS translations+SwissProtein+SPupdate+PIR. Details of these programs can be found at the National Center for Biotechnology Information (see also Altschul et al., “J. Mol. Biol. 215:403-410 (1990)).

Methods of generating and assaying the engineered aldehyde dehydrogenases described herein are well known to one of skill in the art. Examples of such methods are described in the Examples provided herein. Any of a variety of methods can be used to generate an engineered aldehyde dehydrogenase disclosed herein. Such methods include, but are not limited to, site-directed mutagenesis, random mutagenesis, combinatorial libraries, and other mutagenesis methods described herein (see, e.g., Sambrook et al., Molecular Cloning: A Laboratory Manual, Third Ed., Cold Spring Harbor Laboratory, New York (2001); Ausubel et al., Current Protocols in Molecular Biology, John Wiley and Sons, Baltimore, MD (1999); Gillman et al., Directed Evolution Library Creation: Methods and Protocols (Methods in Molecular Biology) Springer, 2nd ed (2014)). One non-limiting example of a method for preparing an engineered aldehyde dehydrogenase is to express recombinant nucleic acids encoding the engineered aldehyde dehydrogenase in a suitable microbial organism, such as a bacterial cell, a yeast cell, or other suitable cell, using methods well known in the art.

In some embodiments, an engineered aldehyde dehydrogenase provided herein is an isolated aldehyde dehydrogenase. An isolated engineered aldehyde dehydrogenase provided herein can be isolated by a variety of methods well-known in the art, for example, recombinant expression systems, precipitation, gel filtration, ion-exchange, reverse-phase and affinity chromatography, and the like. Other well-known methods are described in Deutscher et al., Guide to Protein Purification: Methods in Enzymology, Vol. 182, (Academic Press, (1990)). Alternatively, the isolated polypeptides of the present disclosure can be obtained using well-known recombinant methods (see, e.g., Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, MD (1999)). The methods and conditions for biochemical purification of a polypeptide described herein can be chosen by those skilled in the art, and purification monitored, for example, by a functional assay.

In some embodiments, the provided herein is a recombinant nucleic acid that has a nucleotide sequence encoding an engineered aldehyde dehydrogenase described herein. Accordingly, in some embodiments, provided herein is a recombinant nucleic acid selected from (a) a nucleic acid molecule encoding an engineered aldehyde dehydrogenase that is a variant of a wild-type aldehyde dehydrogenase (SEQ ID NO: 1) or a parent aldehyde dehydrogenase (SEQ ID NO: 3), such as an engineered aldehyde dehydrogenase having one or more alterations at a position described in TABLE 2; (b) a recombinant nucleic acid that hybridizes to an isolated nucleic acid of (a) under highly stringent hybridization conditions; and (c) a recombinant nucleic acid that is complementary to (a) or (b).

In some embodiments, provided herein is a recombinant nucleic acid encoding an engineered aldehyde dehydrogenase that is a variant of a reference polypeptide, wherein the reference polypeptide has an amino acid sequence of SEQ ID NO: 3, and the engineered aldehyde dehydrogenase has one or more alterations at a position described in TABLE 2 relative to SEQ ID NO: 3. In some embodiments, the recombinant nucleic acid encodes an engineered aldehyde dehydrogenase that includes one or more amino acid alterations at a residue corresponding to position 142, 243, 277, 401, 435, or 442, or a combination thereof, in SEQ ID NO: 3. In some embodiments, the recombinant nucleic acid encodes an engineered aldehyde dehydrogenase that includes one or more amino acid alterations at a residue corresponding to position 435 or 442, or a combination thereof, in SEQ ID NO: 3.

In some embodiments, the recombinant nucleic acid encodes an engineered aldehyde dehydrogenase having one or more alterations described in TABLE 2. Accordingly, in some embodiments, the recombinant nucleic acid encodes an engineered aldehyde dehydrogenase having: a) I or V at a residue corresponding to position 142 in SEQ ID NO: 3; b) C at a residue corresponding to position 146 in SEQ ID NO: 3; c) E at a residue corresponding to position 243 in SEQ ID NO: 3; d) C at a residue corresponding to position 277 in SEQ ID NO: 3; e) F at a residue corresponding to position 401 in SEQ ID NO: 3; f) H, M, Q, or R at a residue corresponding to position 435 in SEQ ID NO: 3; and/or g) F at a residue corresponding to position 442 in SEQ ID NO: 3.

In some embodiments, the recombinant nucleic acid encodes an engineered aldehyde dehydrogenase having: I at a residue corresponding to position 142 in SEQ ID NO: 3.

In some embodiments, the recombinant nucleic acid encodes an engineered aldehyde dehydrogenase having: V at a residue corresponding to position 142 in SEQ ID NO: 3.

In some embodiments, the recombinant nucleic acid encodes an engineered aldehyde dehydrogenase having: C at a residue corresponding to position 146 in SEQ ID NO: 3.

In some embodiments, the recombinant nucleic acid encodes an engineered aldehyde dehydrogenase having: E at a residue corresponding to position 243 in SEQ ID NO: 3.

In some embodiments, the recombinant nucleic acid encodes an engineered aldehyde dehydrogenase having: C at a residue corresponding to position 277 in SEQ ID NO: 3.

In some embodiments, the recombinant nucleic acid encodes an engineered aldehyde dehydrogenase having: F at a residue corresponding to position 401 in SEQ ID NO: 3.

In some embodiments, the recombinant nucleic acid encodes an engineered aldehyde dehydrogenase having: H at a residue corresponding to position 435 in SEQ ID NO: 3.

In some embodiments, the recombinant nucleic acid encodes an engineered aldehyde dehydrogenase having: M at a residue corresponding to position 435 in SEQ ID NO: 3.

In some embodiments, the recombinant nucleic acid encodes an engineered aldehyde dehydrogenase having: Q at a residue corresponding to position 435 in SEQ ID NO: 3.

In some embodiments, the recombinant nucleic acid encodes an engineered aldehyde dehydrogenase having: R at a residue corresponding to position 435 in SEQ ID NO: 33.

In some embodiments, the recombinant nucleic acid encodes an engineered aldehyde dehydrogenase having: F at a residue corresponding to position 442 in SEQ ID NO: 3.

In some embodiments, provided herein is a recombinant nucleic acid that hybridizes under highly stringent hybridization conditions to an isolated nucleic acid encoding an engineered aldehyde dehydrogenase described herein. Accordingly, in some embodiments, the recombinant nucleic acid is an isolated nucleic acid that hybridizes under highly stringent hybridization conditions to a nucleic acid that encodes an engineered aldehyde dehydrogenase that is a variant of a wild-type aldehyde dehydrogenase (SEQ ID NO: 1) or a parent aldehyde dehydrogenase (SEQ ID NO: 3), such as an engineered aldehyde dehydrogenase having one or more alterations at a position described in TABLE 2.

In some embodiments, provided herein is a recombinant nucleic acid encoding an engineered aldehyde dehydrogenase provided herein has an amino acid sequence that is a variant of SEQ ID NO: 3 that includes one or more alterations as described in TABLE 2, wherein the portion, other than the one or more alterations described in TABLE 2, of the engineered aldehyde dehydrogenase has at least 65%, 70%, 75%, 80%, 85%, 90%, 95%, 98% or 99% sequence identity, or is identical, to an amino acid sequence referenced as SEQ ID NO: 3. Accordingly, in some embodiments, a recombinant nucleic acid encodes an engineered aldehyde dehydrogenase that has an amino acid sequence that includes one or more alterations as described in TABLE 2 and the portion, other than the alteration described in TABLE 2, of the engineered aldehyde dehydrogenase has at least 65% identical to SEQ ID NO: 3. In some embodiments, a recombinant nucleic acid encodes an engineered aldehyde dehydrogenase that has an amino acid sequence that includes one or more alterations as described in TABLE 2 and the portion, other than the alteration described in TABLE 2, of the engineered aldehyde dehydrogenase has at least 70% identical to SEQ ID NO: 3. In some embodiments, a recombinant nucleic acid encodes an engineered aldehyde dehydrogenase that has an amino acid sequence that includes one or more alterations as described in TABLE 2 and the portion, other than the alteration described in TABLE 2, of the engineered aldehyde dehydrogenase has at least 75% identical to SEQ ID NO: 3. In some embodiments, a recombinant nucleic acid encodes an engineered aldehyde dehydrogenase that has an amino acid sequence that includes one or more alterations as described in TABLE 2 and the portion, other than the alteration described in TABLE 2, of the engineered aldehyde dehydrogenase has at least 80% identical to SEQ ID NO: 3. In some embodiments, a recombinant nucleic acid encodes an engineered aldehyde dehydrogenase that has an amino acid sequence that includes one or more alterations as described in TABLE 2 and the portion, other than the alteration described in TABLE 2, of the engineered aldehyde dehydrogenase has at least 85% identical to SEQ ID NO: 3. In some embodiments, a recombinant nucleic acid encodes an engineered aldehyde dehydrogenase that has an amino acid sequence that includes one or more alterations as described in TABLE 2 and the portion, other than the alteration described in TABLE 2, of the engineered aldehyde dehydrogenase has at least 90% identical to SEQ ID NO: 3. In some embodiments, a recombinant nucleic acid encodes an engineered aldehyde dehydrogenase that has an amino acid sequence that includes one or more alterations as described in TABLE 2 and the portion, other than the alteration described in TABLE 2, of the engineered aldehyde dehydrogenase has at least 95% identical to SEQ ID NO: 3. In some embodiments, a recombinant nucleic acid encodes an engineered aldehyde dehydrogenase that has an amino acid sequence that includes one or more alterations as described in TABLE 2 and the portion, other than the alteration described in TABLE 2, of the engineered aldehyde dehydrogenase has at least 98% identical to SEQ ID NO: 3. In some embodiments, a recombinant nucleic acid encodes an engineered aldehyde dehydrogenase that has an amino acid sequence that includes one or more alterations as described in TABLE 2 and the portion, other than the alteration described in TABLE 2, of the engineered aldehyde dehydrogenase has at least 99% identical to SEQ ID NO: 3. In some embodiments, a recombinant nucleic acid encodes an engineered aldehyde dehydrogenase that has an amino acid sequence that includes one or more alterations as described in TABLE 2 and the portion, other than the alteration described in TABLE 2, of the engineered aldehyde dehydrogenase is identical to SEQ ID NO: 3.

In some embodiments, provided herein is a recombinant nucleic acid that includes a nucleotide sequence encoding an engineered aldehyde dehydrogenase described herein that is operatively linked to a promoter. Such a promoter can express the engineered aldehyde dehydrogenase in a microbial organism as described herein.

In some embodiments, provided herein is a vector containing a recombinant nucleic acid described herein. In some embodiments, the vector is an expression vector. In some embodiments, the vector comprises double stranded DNA.

A recombinant nucleic acid encoding an engineered aldehyde dehydrogenase described herein also includes a nucleic acid that hybridizes to a nucleic acid disclosed herein or a nucleic acid that hybridizes to a nucleic acid that encodes an amino acid sequence disclosed. Hybridization conditions can include highly stringent, moderately stringent, or low stringency hybridization conditions that are well known to one of skill in the art such as those described herein. Similarly, a recombinant nucleic acid that can be used in the compositions and methods described herein can be described as having a certain percent sequence identity to a nucleic acid disclosed herein or a nucleic acid that hybridizes to a nucleic acid molecule that encodes an amino acid sequence disclosed herein. For example, the nucleic acid can have at least 65%, 70%, 75%, 80%, 85%, 90%, 91%, 92%, 93%, 94%, 95%, 96%, 97%, 98% or 99% sequence identity, or be identical, to a nucleotide described herein.

Stringent hybridization refers to conditions under which hybridized polynucleotides are stable. As known to those of skill in the art, the stability of hybridized polynucleotides is reflected in the melting temperature (Tₘ) of the hybrids. In general, the stability of hybridized polynucleotides is a function of the salt concentration, for example, the sodium ion concentration, and temperature. A hybridization reaction can be performed under conditions of lower stringency, followed by washes of varying, but higher, stringency. Reference to hybridization stringency relates to such washing conditions. Highly stringent hybridization includes conditions that permit hybridization of only those nucleotide sequences that form stable hybridized polynucleotides in 0.018M NaCl at 65° C., for example, if a hybrid is not stable in 0.018M NaCl at 65° C., it will not be stable under high stringency conditions, as contemplated herein. High stringency conditions can be provided, for example, by hybridization in 50% formamide, 5× Denhart's solution, 5×SSPE, 0.2% SDS at 42° C., followed by washing in 0.1×SSPE, and 0.1% SDS at 65° C. Hybridization conditions other than highly stringent hybridization conditions can also be used to describe the nucleotide sequences disclosed herein. For example, the phrase moderately stringent hybridization refers to conditions equivalent to hybridization in 50% formamide, 5× Denhart's solution, 5×SSPE, 0.2% SDS at 42° C., followed by washing in 0.2×SSPE, 0.2% SDS, at 42° C. The phrase low stringency hybridization refers to conditions equivalent to hybridization in 10% formamide, 5× Denhart's solution, 6×SSPE, 0.2% SDS at 22° C., followed by washing in 1×SSPE, 0.2% SDS, at 37° C. Denhart's solution contains 1% Ficoll, 1% polyvinylpyrolidone, and 1% bovine serum albumin (BSA). 20×SSPE (sodium chloride, sodium phosphate, ethylene diamine tetraacetic acid (EDTA)) contains 3M sodium chloride, 0.2M sodium phosphate, and 0.025 M (EDTA). Other suitable low, moderate and high stringency hybridization buffers and conditions are well known to those of skill in the art and are described, for example, in Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, MD (1999).

A recombinant nucleic acid encoding an engineered aldehyde dehydrogenase described herein can have at least a certain sequence identity to a nucleotide sequence disclosed herein. Accordingly, in some aspects described herein, a recombinant nucleic acid encoding an engineered aldehyde dehydrogenase has a nucleotide sequence of at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity, or is identical, to a nucleic acid disclosed herein or a nucleic acid that hybridizes to a nucleic acid that encodes an amino acid sequence disclosed herein.

It is understood that a recombinant nucleic acid described herein or an engineered aldehyde dehydrogenase described here can exclude a wild-type parental sequence, for example a parental sequence, such as SEQ ID NO: 1. One skilled in the art will readily understand the meaning of a parental wild-type sequence based on what is well known in the art. It is further understood that such a recombinant nucleic acid described herein can exclude a nucleotide sequence encoding a naturally occurring amino acid sequence as found in nature. Similarly, an engineered aldehyde dehydrogenase described herein can exclude an amino acid sequence as found in nature. Thus, in some embodiments, the recombinant nucleic acid or engineered aldehyde dehydrogenase described herein is as set forth herein, with the proviso that the encoded amino acid sequence is not the wild-type parental sequence or a naturally occurring amino acid sequence and/or that the nucleotide sequence is not a wild-type or naturally occurring nucleotide sequence. A naturally occurring amino acid or nucleotide sequence is understood by those skilled in the art as relating to a sequence that is found in a naturally occurring organism as found in nature. Thus, a nucleotide or amino acid sequence that is not found in the same state or having the same nucleotide or encoded amino acid sequence as in a naturally occurring organism is included within the meaning of a recombinant nucleotide and/or amino acid sequence described herein. For example, a nucleotide or amino acid sequence that has been altered at one or more nucleotide or amino acid positions from a parent sequence, including variants as described herein, are included within the meaning of a nucleotide or amino acid sequence described herein that is not naturally occurring. A recombinant nucleic acid described herein excludes a naturally occurring chromosome that contains the nucleotide sequence, and can further exclude other molecules, as found in a naturally occurring cell, such as DNA binding proteins, for example, proteins such as histones that bind to chromosomes within a eukaryotic cell.

Thus, a recombinant nucleic acid described here has physical and chemical differences compared to a naturally occurring nucleic acid. A recombinant or non-naturally occurring nucleic acid described herein does not contain or does not necessarily have some or all of the chemical bonds, either covalent or non-covalent bonds, of a naturally occurring nucleic acid as found in nature. A recombinant nucleic acid described herein thus differs from a naturally occurring nucleic acid, for example, by having a different chemical structure than a naturally occurring nucleic acid as found in a chromosome. A different chemical structure can occur, for example, by cleavage of phosphodiester bonds that release a recombinant nucleic acid from a naturally occurring chromosome. A recombinant nucleic acid described herein can also differ from a naturally occurring nucleic acid by isolating or separating the nucleic acid from proteins that bind to chromosomal DNA in either prokaryotic or eukaryotic cells, thereby differing from a naturally occurring nucleic acid by different non-covalent bonds. With respect to nucleic acids of prokaryotic origin, a non-naturally occurring nucleic acid described herein does not necessarily have some or all of the naturally occurring chemical bonds of a chromosome, for example, binding to DNA binding proteins such as polymerases or chromosome structural proteins, or is not in a higher order structure such as being supercoiled. With respect to nucleic acids of eukaryotic origin, a non-naturally occurring nucleic acid described herein also does not contain the same internal nucleic acid chemical bonds or chemical bonds with structural proteins as found in chromatin. For example, a non-naturally occurring nucleic acid described herein is not chemically bonded to histones or scaffold proteins and is not contained in a centromere or telomere. Thus, the non-naturally occurring nucleic acids described herein are chemically distinct from a naturally occurring nucleic acid because they either lack or contain different van der Waals interactions, hydrogen bonds, ionic or electrostatic bonds, and/or covalent bonds from a nucleic acid as found in nature. Such differences in bonds can occur either internally within separate regions of the nucleic acid (that is cis) or such difference in bonds can occur in trans, for example, interactions with chromosomal proteins. In the case of a nucleic acid of eukaryotic origin, a cDNA is considered to be a recombinant or non-naturally occurring nucleic acid since the chemical bonds within a cDNA differ from the covalent bonds, that is the sequence, of a gene on chromosomal DNA. Thus, it is understood by those skilled in the art that recombinant or non-naturally occurring nucleic acid is distinct from a naturally occurring nucleic acid.

In some embodiments, provided herein is a method of constructing a host strain that can include, among other steps, introducing a vector disclosed herein into a microbial organism, for example, that is capable of expressing an amino acid sequence encoded by the vector and/or is capable of fermentation. Vectors described herein can be introduced stably or transiently into a microbial organism using techniques well known in the art including, but not limited to, conjugation, electroporation, chemical transformation, transduction, transfection, and ultrasound transformation. Additional methods are disclosed herein, any one of which can be used in the method described herein.

In some embodiments, provided herein is a microbial organism, in particular a non-naturally occurring microbial organism, that expresses an engineered aldehyde dehydrogenase described herein, that is, an engineered aldehyde dehydrogenase described herein. Thus, provided herein is a non-naturally occurring microbial organism having a recombinant nucleic acid encoding an engineered aldehyde dehydrogenase described herein. Accordingly, in some embodiments, provided herein is microbial organism (e.g., host microbial organism) that has a recombinant nucleic acid encoding an engineered aldehyde dehydrogenase that is a variant of a wild-type aldehyde dehydrogenase (SEQ ID NO: 1) or a parent aldehyde dehydrogenase (SEQ ID NO: 3), such as an engineered aldehyde dehydrogenase having one or more alterations at a position described in TABLE 2.

In some embodiments, provided herein is microbial organism (e.g., host microbial organism) that has a recombinant nucleic acid encoding an engineered aldehyde dehydrogenase that is a variant of a reference polypeptide, wherein the reference polypeptide has an amino acid sequence of SEQ ID NO: 3, and the engineered aldehyde dehydrogenase has one or more alterations at a position described in TABLE 2 relative to SEQ ID NO: 3. In some embodiments, the microbial organism has a recombinant nucleic acid that encodes an engineered aldehyde dehydrogenase that includes one or more amino acid alterations at a residue corresponding to position 142, 243, 277, 401, 435, or 442, or a combination thereof, in SEQ ID NO: 3. In some embodiments, the microbial organism has a recombinant nucleic acid that encodes an engineered aldehyde dehydrogenase that includes one or more amino acid alterations at a residue corresponding to position 435 or 442, or a combination thereof, in SEQ ID NO: 3.

In some embodiments, provided herein is microbial organism (e.g., host microbial organism) that has a recombinant nucleic acid encoding an engineered aldehyde dehydrogenase having one or more alterations described in TABLE 2. Accordingly, in some embodiments, the microbial organism has a recombinant nucleic acid that encodes an engineered aldehyde dehydrogenase having: a) I or V at a residue corresponding to position 142 in SEQ ID NO: 3; b) C at a residue corresponding to position 146 in SEQ ID NO: 3; c) E at a residue corresponding to position 243 in SEQ ID NO: 3; d) C at a residue corresponding to position 277 in SEQ ID NO: 3; e) F at a residue corresponding to position 401 in SEQ ID NO: 3; f) H, M, Q, or R at a residue corresponding to position 435 in SEQ ID NO: 3; and/or g) F at a residue corresponding to position 442 in SEQ ID NO: 3

In some embodiments, the microbial organism has a recombinant nucleic acid that encodes an engineered aldehyde dehydrogenase having: I at a residue corresponding to position 142 in SEQ ID NO: 3.

In some embodiments, the microbial organism has a recombinant nucleic acid that encodes an engineered aldehyde dehydrogenase having: V at a residue corresponding to position 142 in SEQ ID NO: 3.

In some embodiments, the microbial organism has a recombinant nucleic acid that encodes an engineered aldehyde dehydrogenase having: C at a residue corresponding to position 146 in SEQ ID NO: 3.

In some embodiments, the microbial organism has a recombinant nucleic acid that encodes an engineered aldehyde dehydrogenase having: E at a residue corresponding to position 243 in SEQ ID NO: 3.

In some embodiments, the microbial organism has a recombinant nucleic acid that encodes an engineered aldehyde dehydrogenase having: C at a residue corresponding to position 277 in SEQ ID NO: 3.

In some embodiments, the microbial organism has a recombinant nucleic acid that encodes an engineered aldehyde dehydrogenase having: F at a residue corresponding to position 401 in SEQ ID NO: 3.

In some embodiments, the microbial organism has a recombinant nucleic acid that encodes an engineered aldehyde dehydrogenase having: H at a residue corresponding to position 435 in SEQ ID NO: 3.

In some embodiments, the microbial organism has a recombinant nucleic acid that encodes an engineered aldehyde dehydrogenase having: M at a residue corresponding to position 435 in SEQ ID NO: 3.

In some embodiments, the microbial organism has a recombinant nucleic acid that encodes an engineered aldehyde dehydrogenase having: Q at a residue corresponding to position 435 in SEQ ID NO: 3.

In some embodiments, the microbial organism has a recombinant nucleic acid that encodes an engineered aldehyde dehydrogenase having: R at a residue corresponding to position 435 in SEQ ID NO: 33.

In some embodiments, the microbial organism has a recombinant nucleic acid that encodes an engineered aldehyde dehydrogenase having: F at a residue corresponding to position 442 in SEQ ID NO: 3.

In some embodiments, provided herein is microbial organism (e.g., host microbial organism) that has a recombinant nucleic acid encoding an engineered aldehyde dehydrogenase an engineered aldehyde dehydrogenase provided herein has an amino acid sequence that is a variant of SEQ ID NO: 3 that includes one or more alterations as described in TABLE 2, wherein the portion, other than the one or more alterations described in TABLE 2, of the engineered aldehyde dehydrogenase has at least 65%, 70%, 75%, 80%, 85%, 90%, 95%, 98% or 99% sequence identity, or is identical, to an amino acid sequence referenced as SEQ ID NO: 3. Accordingly, in some embodiments, provided herein is microbial organism (e.g., host microbial organism) that has a recombinant nucleic acid encoding an engineered aldehyde dehydrogenase that has an amino acid sequence that includes one or more alterations as described in TABLE 2 and the portion, other than the alteration described in TABLE 2, of the engineered aldehyde dehydrogenase has at least 65% identical to SEQ ID NO: 3. In some embodiments, provided herein is microbial organism (e.g., host microbial organism) that has a recombinant nucleic acid encodes an engineered aldehyde dehydrogenase that has an amino acid sequence that includes one or more alterations as described in TABLE 2 and the portion, other than the alteration described in TABLE 2, of the engineered aldehyde dehydrogenase has at least 70% identical to SEQ ID NO: 3. In some embodiments, provided herein is microbial organism (e.g., host microbial organism) that has a recombinant nucleic acid encodes an engineered aldehyde dehydrogenase that has an amino acid sequence that includes one or more alterations as described in TABLE 2 and the portion, other than the alteration described in TABLE 2, of the engineered aldehyde dehydrogenase has at least 75% identical to SEQ ID NO: 3. In some embodiments, provided herein is microbial organism (e.g., host microbial organism) that has a recombinant nucleic acid encodes an engineered aldehyde dehydrogenase that has an amino acid sequence that includes one or more alterations as described in TABLE 2 and the portion, other than the alteration described in TABLE 2, of the engineered aldehyde dehydrogenase has at least 80% identical to SEQ ID NO: 3. In some embodiments, provided herein is microbial organism (e.g., host microbial organism) that has a recombinant nucleic acid encodes an engineered aldehyde dehydrogenase that has an amino acid sequence that includes one or more alterations as described in TABLE 2 and the portion, other than the alteration described in TABLE 2, of the engineered aldehyde dehydrogenase has at least 85% identical to SEQ ID NO: 3. In some embodiments, provided herein is microbial organism (e.g., host microbial organism) that has a recombinant nucleic acid encodes an engineered aldehyde dehydrogenase that has an amino acid sequence that includes one or more alterations as described in TABLE 2 and the portion, other than the alteration described in TABLE 2, of the engineered aldehyde dehydrogenase has at least 90% identical to SEQ ID NO: 3. In some embodiments, provided herein is microbial organism (e.g., host microbial organism) that has a recombinant nucleic acid encodes an engineered aldehyde dehydrogenase that has an amino acid sequence that includes one or more alterations as described in TABLE 2 and the portion, other than the alteration described in TABLE 2, of the engineered aldehyde dehydrogenase has at least 95% identical to SEQ ID NO: 3. In some embodiments, provided herein is microbial organism (e.g., host microbial organism) that has a recombinant nucleic acid encodes an engineered aldehyde dehydrogenase that has an amino acid sequence that includes one or more alterations as described in TABLE 2 and the portion, other than the alteration described in TABLE 2, of the engineered aldehyde dehydrogenase has at least 98% identical to SEQ ID NO: 3. In some embodiments, provided herein is microbial organism (e.g., host microbial organism) that has a recombinant nucleic acid encodes an engineered aldehyde dehydrogenase that has an amino acid sequence that includes one or more alterations as described in TABLE 2 and the portion, other than the alteration described in TABLE 2, of the engineered aldehyde dehydrogenase has at least 99% identical to SEQ ID NO: 3. In some embodiments, provided herein is microbial organism (e.g., host microbial organism) that has a recombinant nucleic acid encodes an engineered aldehyde dehydrogenase that has an amino acid sequence that includes one or more alterations as described in TABLE 2 and the portion, other than the alteration described in TABLE 2, of the engineered aldehyde dehydrogenase is identical to SEQ ID NO: 3.

In one embodiment, the cell comprises a pathway that produces 3-hydroxybutyraldehyde (3-HBal) and/or 1,3-butanediol (1,3-BDO), or an ester or amide thereof. In another embodiment, the cell comprises a pathway that produces 4-hydroxybutyraldehyde (4-HBal) and/or 1,4-butanediol (1,4-BDO), or an ester or amide thereof. In one embodiment, the cell is capable of fermentation. In one embodiment, the cell further includes at least one substrate for the engineered aldehyde dehydrogenase described herein present or produced in the cell. In some embodiments, the substrate is 3-hydroxybutyryl-CoA (3-HB-CoA). In some embodiments, the substrate is (R)-3-hydroxybutyryl-CoA (R-3-HB-CoA). In some embodiments, the cell has higher activity for R-3-HB-CoA over (S)-3-hydroxybutyryl-CoA (S-3-HB-CoA). In some embodiments, the substrate is 4-hydroxybutyryl-CoA (4-HB-CoA). Also provided herein is a culture medium comprising a cell described herein.

The engineered aldehyde dehydrogenase described herein can be utilized in a pathway that converts an acyl-CoA to its corresponding aldehyde. Exemplary pathways for 3-HBal and/or 1,3-BDO that comprise an aldehyde dehydrogenase have been described, for example, in WO 2010/127319, WO 2013/036764, U.S. Pat. No. 9,017,983, US 2013/0066035, each of which is incorporated herein by reference.

Exemplary 3-HBal and/or 1,3-BDO pathways are described in WO 2010/127319, WO 2013/036764, U.S. Pat. No. 9,017,983 and US 2013/0066035. Such a 3-HBal and/or 1,3-BDO pathway that comprises an aldehyde dehydrogenase includes, for example, (G) acetoacetyl-CoA reductase (ketone reducing); (H) 3-hydroxybutyryl-CoA reductase (aldehyde forming), also referred to herein as 3-hydroxybutyraldehyde dehydrogenase, an aldehyde dehydrogenase (ALD); and (C) 3-hydroxybutyraldehyde reductase, also referred to herein as a 1,3-BDO dehydrogenase. Acetoacetyl-CoA can be formed by converting two molecules of acetyl-CoA into one molecule of acetoacetyl-CoA employing a thiolase. Acetoacetyl-CoA thiolase converts two molecules of acetyl-CoA into one molecule each of acetoacetyl-CoA and CoA (see, e.g., WO 2013/036764 and US 2013/0066035).

An exemplary 1,3-BDO pathway is shown in FIG. 2 of WO 2010/127319. Briefly, acetoacetyl-CoA can be converted to 3-hydroxybutyryl-CoA by acetoacetyl-CoA reductase (ketone reducing) (EC 1.1.1.a) (step G of FIG. 2). 3-Hydroxybutyryl-CoA can be converted to 3-hydroxybutyraldehyde by 3-hydroxybutyryl-CoA reductase (aldehyde forming) (EC 1.2.1.b), also referred to herein as 3-hydroxybutyraldehyde dehydrogenase, including an engineered aldehyde dehydrogenase provided herein (step H of FIG. 2). 3-Hydroxybutyraldehyde can be converted to 1,3-butanediol by 3-hydroxybutyraldehyde reductase (EC 1.1.1.a), also referred to herein as 1,3-BDO dehydrogenase (step C of FIG. 2).

As disclosed herein, an engineered aldehyde dehydrogenase described herein can function in a pathway to convert 3-hydroxybutyryl-CoA to 3-hydroxybutyraldehyde. In the pathway described above that includes an aldehyde dehydrogenase that converts 3-hydroxybutyryl-CoA to 3-hydroxybutyraldehyde, the pathway converts acetoacetyl-CoA to 3-hydroxybutyryl-CoA. An engineered aldehyde dehydrogenase described herein can also be used in other 3-HBal and/or 1,3-BDO pathways that comprise 3-hydroxybutyryl-CoA as a substrate/product in the pathway. One skilled in the art can readily utilize an engineered aldehyde dehydrogenase described herein to convert 3-hydroxybutyryl-CoA to 3-hydroxybutyraldehyde in any desired pathway that comprises such a reaction.

Exemplary 4-HBal and/or 1,4-BDO pathways are described in WO 2008/115840, WO 2010/030711, WO 2010/141920, WO 2011/047101, WO 2013/184602, WO 2014/176514, U.S. Pat. Nos. 8,067,214, 7,858,350, 8,129,169, 8,377,666, US 2013/0029381, US 2014/0030779, US 2015/0148513 and US 2014/0371417. Such a 4-HBal and/or 1,4-BDO pathway that comprises an aldehyde dehydrogenase includes, for example, (1) succinyl-CoA synthetase; (2) CoA-independent succinic semialdehyde dehydrogenase; (3) α-ketoglutarate dehydrogenase; (4) glutamate:succinate semialdehyde transaminase; (5) glutamate decarboxylase; (6) CoA-dependent succinic semialdehyde dehydrogenase; (7) 4-hydroxybutanoate dehydrogenase; (8) α-ketoglutarate decarboxylase; (9) 4-hydroxybutyryl CoA:acetyl-CoA transferase; (10) butyrate kinase (also referred to as 4-hydroxybutyrate kinase); (11) phosphotransbutyrylase (also referred to as phospho-trans-4-hydroxybutyrylase); (12) aldehyde dehydrogenase (also referred to as 4-hydroxybutyryl-CoA reductase); (13) alcohol dehydrogenase, such as 1,4-butanediol dehydrogenase (also referred to as 4-hydroxybutanal reductase or 4-hydroxybutyraldehyde reductase) (see FIG. 2 of WO 2008/115840).

Similar to FIG. 2 of WO 2008/115840, exemplary 1,4-BDO pathways are shown in FIG. 8A of WO 2010/141920. Briefly, succinyl-CoA can be converted to succinic semialdehyde by succinyl-CoA reductase (or succinate semialdehyde dehydrogenase) (EC 1.2.1.b). Succinate semialdehyde can be converted to 4-hydroxybutyrate by 4-hydroxybutyrate dehydrogenase (EC 1.1.1.a). Alternatively, succinyl-CoA can be converted to 4-hydroxybutyrate by succinyl-CoA reductase (alcohol forming) (EC 1.1.1.c). 4-Hydroxybutyrate can be converted to 4-hydroxybutyryl-CoA by 4-hydroxybutyryl-CoA transferase (EC 2.8.3.a), by 4-hydroxybutyryl-CoA hydrolase (EC 3.1.2.a) or by 4-hydroxybutyryl-CoA ligase (or 4-hydroxybutyryl-CoA synthetase) (EC 6.2.1.a). Alternatively, 4-hydroxybutyrate can be converted to 4-hydroxybutyryl-phosphate by 4-hydroxybutyrate kinase (EC 2.7.2.a). 4-Hydroxybutyryl-phosphate can be converted to 4-hydroxybutyryl-CoA by phosphotrans-4-hydroxybutyrylase (EC 2.3.1.a). Alternatively, 4-hydroxybutyryl-phosphate can be converted to 4-hydroxybutanal by 4-hydroxybutanal dehydrogenase (phosphorylating) (EC 1.2.1.d). 4-Hydroxybutyryl-CoA can be converted to 4-hydroxybutanal by 4-hydroxybutyryl-CoA reductase (or 4-hydroxybutanal dehydrogenase) (EC 1.2.1.b), including by an aldehyde dehydrogenase variant provided herein. Alternatively, 4-hydroxybutyryl-CoA can be converted to 1,4-butanediol by 4-hydroxybutyryl-CoA reductase (alcohol forming) (EC 1.1.1.c). 4-Hydroxybutanal can be converted to 1,4-butanediol by 1,4-butanediol dehydrogenase (EC 1.1.1.a).

Exemplary 1,4-BDO pathways are also shown in FIG. 8B of WO 2010/141920. Briefly, alpha-ketoglutarate can be converted to succinic semialdehyde by alpha-ketoglutarate decarboxylase (EC 4.1.1.a). Alternatively, alpha-ketoglutarate can be converted to glutamate by glutamate dehydrogenase (EC 1.4.1.a). 4-Aminobutyrate can be converted to succinic semialdehyde by 4-aminobutyrate oxidoreductase (deaminating) (EC 1.4.1.a) or 4-aminobutyrate transaminase (EC 2.6.1.a). Glutamate can be converted to 4-aminobutyrate by glutamate decarboxylase (EC 4.1.1.a). Succinate semialdehyde can be converted to 4-hydroxybutyrate by 4-hydroxybutyrate dehydrogenase (EC 1.1.1.a). 4-Hydroxybutyrate can be converted to 4-hydroxybutyryl-CoA by 4-hydroxybutyryl-CoA transferase (EC 2.8.3.a), by 4-hydroxybutyryl-CoA hydrolase (EC 3.1.2.a), or by 4-hydroxybutyryl-CoA ligase (or 4-hydroxybutyryl-CoA synthetase) (EC 6.2.1.a). 4-Hydroxybutyrate can be converted to 4-hydroxybutyryl-phosphate by 4-hydroxybutyrate kinase (EC 2.7.2.a). 4-Hydroxybutyryl-phosphate can be converted to 4-hydroxybutyryl-CoA by phosphotrans-4-hydroxybutyrylase (EC 2.3.1.a). Alternatively, 4-hydroxybutyryl-phosphate can be converted to 4-hydroxybutanal by 4-hydroxybutanal dehydrogenase (phosphorylating) (EC 1.2.1.d). 4-Hydroxybutyryl-CoA can be converted to 4-hydroxybutanal by 4-hydroxybutyryl-CoA reductase (or 4-hydroxybutanal dehydrogenase) (EC 1.2.1.b), including by an engineered aldehyde dehydrogenase provided herein. 4-Hydroxybutyryl-CoA can be converted to 1,4-butanediol by 4-hydroxybutyryl-CoA reductase (alcohol forming) (EC 1.1.1.c). 4-Hydroxybutanal can be converted to 1,4-butanediol by 1,4-butanediol dehydrogenase (EC 1.1.1.a).

As disclosed herein, an engineered aldehyde dehydrogenase provided herein can function in a pathway to convert 4-hydroxybutyryl-CoA to 4-hydroxybutyraldehyde. In the pathways described above that comprise an aldehyde dehydrogenase that converts 4-hydroxybutyryl-CoA to 4-hydroxybutyraldehyde, the pathways convert 4-hydroxybutyrate to 4-hydroxybutyryl-CoA or 4-hydroxybutyryl phosphate to 4-hydroxybutyryl-CoA (see FIG. 2 of WO 2008/115840). An engineered aldehyde dehydrogenase provided herein can also be used in other 4-HBal and/or 1,4-BDO pathways that comprise 4-hydroxybutyryl-CoA as a substrate/product in the pathway. One skilled in the art can readily utilize an engineered aldehyde dehydrogenase provided herein to convert 4-hydroxybutyryl-CoA to 4-hydroxybutyraldehyde in any desired pathway that comprises such a reaction. For example, 4-oxobutyryl-CoA can be converted to 4-hydroxybutyryl-CoA as described and shown in WO 2010/141290, FIG. 9A. In addition, 5-hydroxy-2-oxopentanoic acid can be converted to 4-hydroxybutyryl-CoA as described and shown in WO 2010/141290, FIGS. 10 and 11. Also, acetoacetyl-CoA, 3-hydroxybutyryl-CoA, crotonyl-CoA and/or vinylacetyl-CoA can be converted to 4-hydroxybutyryl-CoA as described and shown in WO 2010/141290, FIG. 12. Additionally, 4-hydroxybut-2-enoyl-CoA can be converted to 4-hydroxybutyryl-CoA as described and shown in WO 2010/141290, FIG. 13. Thus, one skilled in the art will readily understand how to use an engineered aldehyde dehydrogenase provided herein in a 4-HBal and/or 1,4-BDO pathway that comprises conversion of 4-hydroxybutyryl-CoA to 4-hydroxybutyraldehyde, as desired.

Enzyme types required to convert common central metabolic intermediates into 1,3-BDO or 1,4-BDO are indicated above with representative Enzyme Commission (EC) numbers (see also WO 2010/127319, WO 2013/036764, WO 2008/115840, WO 2010/030711, WO 2010/141920, WO 2011/047101, WO 2013/184602, WO 2014/176514, U.S. Pat. Nos. 9,017,983, 8,067,214, 7,858,350, 8,129,169, 8,377,666, US 2013/0066035, US 2013/0029381, US 2014/0030779, US 2015/0148513, and US 2014/0371417). The first three digits of each label correspond to the first three Enzyme Commission number digits which denote the general type of transformation independent of substrate specificity. Exemplary enzymes include: 1.1.1.a, Oxidoreductase (ketone to hydroxyl or aldehyde to alcohol); 1.1.1.c, Oxidoreductase (2 step, acyl-CoA to alcohol); 1.2.1.b, Oxidoreductase (acyl-CoA to aldehyde); 1.2.1.c, Oxidoreductase (2-oxo acid to acyl-CoA, decarboxylation); 1.2.1.d, Oxidoreductase (phosphorylating/dephosphorylating); 1.3.1.a, Oxidoreductase operating on CH-CH donors; 1.4.1.a, Oxidoreductase operating on amino acids (deaminating); 2.3.1.a, Acyltransferase (transferring phosphate group); 2.6.1.a, Aminotransferase; 2.7.2.a, Phosphotransferase, carboxyl group acceptor; 2.8.3.a, Coenzyme-A transferase; 3.1.2.a, Thiolester hydrolase (CoA specific); 4.1.1.a, Carboxy-lyase; 4.2.1.a, Hydro-lyase; 4.3.1.a, Ammonia-lyase; 5.3.3.a, Isomerase; 5.4.3.a, Aminomutase; and 6.2.1.a, Acid-thiol ligase.

An engineered aldehyde dehydrogenase described herein can be utilized in a cell or in vitro to convert an acyl-CoA to its corresponding aldehyde. As disclosed herein, the engineered aldehyde dehydrogenases described herein have beneficial and useful properties, including but not limited to increased specificity for the R enantiomer of 3-hydroxybutyryl-CoA over the S enantiomer, increased specificity for 3-hydroxybutyryl-CoA and/or 4-hydroxybutyryl-CoA over acetyl-CoA, increased activity, decreased by-product production, and the like. Engineered aldehyde dehydrogenases described herein can be used to produce the R-form of 1,3-butanediol (also referred to as (R)-1,3-butanediol), by enzymatically converting the product of an engineered aldehyde dehydrogenase described herein, (R)-3-hydroxybutyraldehyde, to (R)-1,3-butanediol using a 1,3-butanediol dehydrogenase.

The bio-derived R-form of 1,3-butanediol can be utilized for production of downstream products for which the R-form is preferred. In some embodiments, the R-form can be utilized as a pharmaceutical and/or nutraceutical (see, e.g., WO 2014/190251). For example, (R)-1,3-butanediol can be used to produce (3R)-hydroxybutyl (3R)-hydroxybutyrate, which can have beneficial effects such as increasing the level of ketone bodies in the blood. Increasing the level of ketone bodies can lead to various clinical benefits, including an enhancement of physical and cognitive performance and treatment of cardiovascular conditions, diabetes and treatment of mitochondrial dysfunction disorders and in treating muscle fatigue and impairment (see WO 2014/190251). The bio-derived R-form of 1,3-butanediol can be utilized for production of downstream products in which a non-petroleum based product is desired, for example, by substituting petroleum-derived racemate 1,3-butanediol, its S-form or its R-form, with the bio-derived R-form.

In some embodiments, provided herein is 3-HBal or 1,3-BDO, or downstream products related thereto, such as an ester or amide thereof, enantiomerically enriched for the R form of the compound. In some embodiments, the 3-HBal or 1,3-BDO is a racemate enriched in R-enantiomer, that is, includes more R-enantiomer than S-enantiomer. For example, the 3-HBal or 1,3-BDO racemate can include 55% or more R-enantiomer and 45% or less S-enantiomer. For example, the 3-HBal or 1,3-BDO racemate can include 60% or more R-enantiomer and 40% or less S-enantiomer. For example, the 3-HBal or 1,3-BDO racemate can include 65% or more R-enantiomer and 35% or less S-enantiomer. For example, the 3-HBal or 1,3-BDO racemate can include 70% or more R-enantiomer and 30% or less S-enantiomer. For example, the 3-HBal or 1,3-BDO racemate can include 75% or more R-enantiomer and 25% or less S-enantiomer. For example, the 3-HBal or 1,3-BDO racemate can include 80% or more R-enantiomer and 20% or less S-enantiomer. For example, the 3-HBal or 1,3-BDO racemate can include 85% or more R-enantiomer and 15% or less S-enantiomer. For example, the 3-HBal or 1,3-BDO racemate can include 90% or more R-enantiomer and 10% or less S-enantiomer. For example, the 3-HBal or 1,3-BDO racemate can include 95% or more R-enantiomer and 5% or less S-enantiomer. In some embodiments, the 3-HBal or 1,3-BDO, or downstream products related thereto such as an ester or amide thereof, is greater than 90% R form, for example, greater than 95%, 96%, 97%, 98%, 99% or 99.9% R form. In one embodiment, the 3-HBal and/or 1,3-BDO, or downstream products related thereto, such as an ester or amide thereof, is ≥55% R-enantiomer, ≥60% R-enantiomer, ≥65% R-enantiomer, ≥70% R-enantiomer, ≥75% R-enantiomer, ≥80% R-enantiomer, ≥85% R-enantiomer, ≥90% R-enantiomer, or ≥95% R-enantiomer, and can be highly chemically pure, e.g., ≥99%, for example, ≥95%, ≥96%, ≥97%, ≥98%, ≥99%, ≥99.1%, ≥99.2%, ≥99.3%, ≥99.4%, ≥99.5%, ≥99.6%, ≥99.7%, 99.8% or ≥99.9% R-enantiomer.

In one embodiment, a petroleum-derived racemic mixture of a precursor of 3-HBal and/or 1,3-BDO, in particular a racemic mixture of 3-hydroxybutyryl-CoA, is used as a substrate for an engineered aldehyde dehydrogenase provided herein, which exhibits increased specificity for the R form over the S form, to produce 3-HBal or 1,3-BDO, or a downstream product related thereto such as an ester or amide thereof, that is enantiomerically enriched for the R form. Such a reaction can be carried out by feeding a petroleum-derived precursor to a cell that expresses an engineered aldehyde dehydrogenase provided herein, in particular a cell that can convert the precursor to 3-hydroxybutyryl-CoA, or can be carried out in vitro using one or more enzymes to convert the petroleum-derived precursor to 3-hydroxybutyryl-CoA, or a combination of in vivo and in vitro reactions. A reaction to produce 4-hydroxybutyryl-CoA with an engineered aldehyde dehydrogenase provided herein can similarly be carried out by feeding a petroleum-derived precursor to a cell that expresses an engineered aldehyde dehydrogenase provided herein, in particular a cell that can convert the precursor to 4-hydroxybutyryl-CoA, or can be carried out in vitro using one or more enzymes to convert the petroleum-derived precursor to 4-hydroxybutyryl-CoA, or a combination of in vivo and in vitro reactions.

While generally described herein as a cell that contains a 3-HBal, 1,3-BDO, 4-HBal or 1,4-BDO pathway comprising an engineered aldehyde dehydrogenase provided herein, it is understood that provided herein is a cell comprising at least one recombinant nucleic acid encoding an engineered aldehyde dehydrogenase provided herein. The aldehyde dehydrogenase can be expressed in a sufficient amount to produce a desired product, such a product of a 3-HBal, 1,3-BDO, 4-HBal or 1,4-BDO pathway, or a downstream product related thereto such as an ester or amide thereof. Exemplary 3-HBal, 1,3-BDO, 4-HBal or 1,4-BDO pathways are described herein.

It is understood that any of the pathways disclosed herein, as described in the Examples, including the pathways described herein, can be utilized to generate a cell that produces any pathway intermediate or product, as desired, in particular a pathway that utilizes an engineered aldehyde dehydrogenase provided herein. As disclosed herein, such a cell that produces an intermediate can be used in combination with another cell expressing one or more upstream or downstream pathway enzymes to produce a desired product. However, it is understood that a cell that produces a 3-HBal, 1,3-BDO, 4-HBal or 1,4-BDO pathway intermediate can be utilized to produce the intermediate as a desired product.

The subject matter described herein includes general reference to the metabolic reaction, reactant or product thereof, or with specific reference to one or more nucleic acids or genes encoding an enzyme associated with or catalyzing, or a protein associated with, the referenced metabolic reaction, reactant or product. Unless otherwise expressly stated herein, those skilled in the art will understand that reference to a reaction also constitutes reference to the reactants and products of the reaction. Similarly, unless otherwise expressly stated herein, reference to a reactant or product also references the reaction, and reference to any of these metabolic constituents also references the gene or genes encoding the enzymes that catalyze or proteins involved in the referenced reaction, reactant or product. Likewise, given the well known fields of metabolic biochemistry, enzymology and genomics, reference herein to a gene or encoding nucleic acid also constitutes a reference to the corresponding encoded enzyme and the reaction it catalyzes or a protein associated with the reaction as well as the reactants and products of the reaction.

The cells provided herein can be produced by introducing an expressible nucleic acid encoding an engineered aldehyde dehydrogenase provided herein, and optionally expressible nucleic acids encoding one or more of the enzymes or proteins participating in one or more 3-HBal, 1,3-BDO, 4-HBal or 1,4-BDO biosynthetic pathways, and further optionally a nucleic acid encoding an enzyme that produces a downstream product related to 3-HBal, 1,3-BDO, 4-HBal or 1,4-BDO such as an ester or amide thereof. Depending on the host cell chosen, nucleic acids for some or all of a particular 3-HBal, 1,3-BDO, 4-HBal or 1,4-BDO biosynthetic pathway, or downstream product, can be expressed. For example, if a chosen host is deficient in one or more enzymes or proteins for a desired biosynthetic pathway, then expressible nucleic acids for the deficient enzyme(s) or protein(s) are introduced into the host for subsequent exogenous expression. Alternatively, if the chosen host exhibits endogenous expression of some pathway genes, but is deficient in others, then an encoding nucleic acid is included for the deficient enzyme(s) or protein(s) to achieve 3-HBal, 1,3-BDO, 4-HBal or 1,4-BDO biosynthesis, or exogenous expression of endogenously expressed genes can be provided to increase expression of pathway enzymes, if desired. Thus, a cell provided herein can be produced by introducing an engineered aldehyde dehydrogenase provided herein, and optionally exogenous enzyme or protein activities to obtain a desired biosynthetic pathway, or by introducing one or more exogenous enzyme or protein activities, including an engineered aldehyde dehydrogenase provided herein that, together with one or more endogenous enzymes or proteins, produces a desired product such as 3-HBal, 1,3-BDO, 4-HBal or 1,4-BDO, or a downstream product related thereto such as an ester or amide thereof.

Host cells can be selected from, and the non-naturally cells expressing an engineered aldehyde dehydrogenase provided herein generated in, for example, bacteria, yeast, fungus or any of a variety of microorganisms applicable or suitable to fermentation processes. In some embodiments, the microbial organism is a species of bacteria, yeast or fungus. In some embodiments, the microbial organism is a species of bacteria. In some embodiments, the microbial organism is a species of yeast. In some embodiments, the microbial organism is a species of fungus. Exemplary bacteria include any species selected from the order Enterobacteriales, family Enterobacteriaceae, including the genera Escherichia and Klebsiella; the order Aeromonadales, family Succinivibrionaceae, including the genus Anaerobiospirillum; the order Pasteurellales, family Pasteurellaceae, including the genera Actinobacillus and Mannheimia; the order Rhizobiales, family Bradyrhizobiaceae, including the genus Rhizobium; the order Bacillales, family Bacillaceae, including the genus Bacillus; the order Actinomycetales, families Corynebacteriaceae and Streptomycetaceae, including the genus Corynebacterium and the genus Streptomyces, respectively; order Rhodospirillales, family Acetobacteraceae, including the genus Gluconobacter; the order Sphingomonadales, family Sphingomonadaceae, including the genus Zymomonas; the order Lactobacillales, families Lactobacillaceae and Streptococcaceae, including the genus Lactobacillus and the genus Lactococcus, respectively; the order Clostridiales, family Clostridiaceae, genus Clostridium; and the order Pseudomonadales, family Pseudomonadaceae, including the genus Pseudomonas. Non-limiting species of host bacteria include Escherichia coli, Klebsiella oxytoca, Anaerobiospirillum succiniciproducens, Actinobacillus succinogenes, Mannheimia succiniciproducens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, Clostridium acetobutylicum, Pseudomonas fluorescens, and Pseudomonas putida. E. coli is a particularly useful host organism since it is a well characterized microbial organism suitable for genetic engineering.

Similarly, exemplary species of yeast or fungi species include any species selected from the order Saccharomycetales, family Saccaromycetaceae, including the genera Saccharomyces, Kluyveromyces and Pichia; the order Saccharomycetales, family Dipodascaceae, including the genus Yarrowia; the order Schizosaccharomycetales, family Schizosaccaromycetaceae, including the genus Schizosaccharomyces; the order Eurotiales, family Trichocomaceae, including the genus Aspergillus; and the order Mucorales, family Mucoraceae, including the genus Rhizopus. Non-limiting species of host yeast or fungi include Saccharomyces cerevisiae, Schizosaccharomyces pombe, Kluyveromyces lactis, Kluyveromyces marxianus, Aspergillus terreus, Aspergillus niger, Pichia pastoris, Rhizopus arrhizus, Rhizopus oryzae, Yarrowia lipolytica, and the like. A particularly useful host organism that is a yeast includes Saccharomyces cerevisiae.

Although generally described herein as utilizing a cell that is a microbial organism as a host cell, particularly for producing 3-HBal, 1,3-BDO, 4-HBal or 1,4-BDO, or a downstream product related thereto such as an ester or amide thereof, it is understood that a host cell can be a cell line of a higher eukaryote, such as a mammalian cell line or insect cell line. Thus, it is understood that reference herein to a host cell that is a microbial organism can alternatively utilize a higher eukaryotic cell line to produce a desired product. Exemplary higher eukaryotic cell lines include, but are not limited to, Chinese hamster ovary (CHO), human (Hela, Human Embryonic Kidney (HEK) 293, Jurkat), mouse (3T3), primate (Vero), insect (Sf9), and the like. Such cell lines are commercially available (see, for example, the American Type Culture Collection (ATCC; Manassas VA); Life Technologies, Carlsbad CA). It is understood that any suitable host cell can be used to introduce an engineered aldehyde dehydrogenase provided herein, and optionally metabolic and/or genetic modifications to produce a desired product.

Depending on the 3-HBal, 1,3-BDO, 4-HBal or 1,4-BDO biosynthetic pathway constituents of a selected host cell, the non-naturally occurring cells provided herein will include at least one exogenously expressed 3-HBal, 1,3-BDO, 4-HBal or 1,4-BDO pathway-encoding nucleic acid and up to all encoding nucleic acids for one or more 3-HBal, 1,3-BDO, 4-HBal or 1,4-BDO biosynthetic pathways, or a downstream product related thereto such as an ester or amide thereof, including an engineered aldehyde dehydrogenase provided herein. For example, 3-HBal, 1,3-BDO, 4-HBal or 1,4-BDO biosynthesis can be established in a host deficient in a pathway enzyme or protein through exogenous expression of the corresponding encoding nucleic acid, including an engineered aldehyde dehydrogenase provided herein. In a host deficient in all enzymes or proteins of a 3-HBal, 1,3-BDO, 4-HBal or 1,4-BDO pathway, or a downstream product related thereto such as an ester or amide thereof, exogenous expression of all enzyme or proteins in the pathway can be included, although it is understood that all enzymes or proteins of a pathway can be expressed even if the host contains at least one of the pathway enzymes or proteins. For example, exogenous expression of all enzymes or proteins in a pathway for production of 3-HBal, 1,3-BDO, 4-HBal or 1,4-BDO pathway, or a downstream product related thereto such as an ester or amide thereof, can be included, including an engineered aldehyde dehydrogenase provided herein.

Given the teachings and guidance provided herein, those skilled in the art will understand that the number of encoding nucleic acids to introduce in an expressible form will, at least, parallel the 3-HBal, 1,3-BDO, 4-HBal or 1,4-BDO pathway deficiencies of the selected host cell if a 3-HBal, 1,3-BDO, 4-HBal or 1,4-BDO pathway is to be included in the cell. Therefore, a non-naturally occurring cell provided herein can have one, two, three, four, five, six, seven, eight, and so forth, depending on the particular pathway, up to all nucleic acids encoding the enzymes or proteins constituting a 3-HBal, 1,3-BDO, 4-HBal or 1,4-BDO biosynthetic pathway disclosed herein. In some embodiments, the non-naturally occurring cells also can include other genetic modifications that facilitate or optimize 3-HBal, 1,3-BDO, 4-HBal or 1,4-BDO biosynthesis or that confer other useful functions onto the host cell. One such other functionality can include, for example, augmentation of the synthesis of one or more of the 3-HBal, 1,3-BDO, 4-HBal or 1,4-BDO pathway precursors such acetyl-CoA or acetoacetyl-CoA.

Generally, a host cell is selected such that it can express an engineered aldehyde dehydrogenase provided herein, and optionally produces the precursor of a 3-HBal, 1,3-BDO, 4-HBal or 1,4-BDO pathway, in a cell containing such a pathway, either as a naturally produced molecule or as an engineered product that either provides de novo production of a desired precursor or increased production of a precursor naturally produced by the host cell. A host organism can be engineered to increase production of a precursor, as disclosed herein. In addition, a cell that has been engineered to produce a desired precursor can be used as a host organism and further engineered to express enzymes or proteins of a 3-HBal, 1,3-BDO, 4-HBal or 1,4-BDO pathway, or a downstream product related thereto such as an ester or amide thereof, if desired.

In some embodiments, a non-naturally occurring cell provided herein is generated from a host that contains the enzymatic capability to synthesize 3-HBal, 1,3-BDO, 4-HBal or 1,4-BDO, or a downstream product related thereto such as an ester or amide thereof. In this specific embodiment it can be useful to increase the synthesis or accumulation of a 3-HBal, 1,3-BDO, 4-HBal or 1,4-BDO pathway product to, for example, drive 3-HBal, 1,3-BDO, 4-HBal or 1,4-BDO pathway reactions toward 3-HBal, 1,3-BDO, 4-HBal or 1,4-BDO production, or a downstream product related thereto such as an ester or amide thereof. Increased synthesis or accumulation can be accomplished by, for example, overexpression of nucleic acids encoding one or more of the above-described 3-HBal, 1,3-BDO, 4-HBal or 1,4-BDO pathway enzymes or proteins, including an engineered aldehyde dehydrogenase provided herein. Overexpression of the enzyme or enzymes and/or protein or proteins of the 3-HBal, 1,3-BDO, 4-HBal or 1,4-BDO pathway can occur, for example, through exogenous expression of the endogenous gene or genes, or through exogenous expression of the heterologous gene or genes, including exogenous expression of an engineered aldehyde dehydrogenase provided herein. Therefore, naturally occurring organisms can be readily converted to non-naturally occurring cells provided herein, for example, producing 3-HBal, 1,3-BDO, 4-HBal or 1,4-BDO or a downstream product related thereto such as an ester or amide thereof, through overexpression of one, two, three, four, five, six, seven, eight, or more, depending on the 3-HBal, 1,3-BDO, 4-HBal or 1,4-BDO pathway, that is, up to all nucleic acids encoding 3-HBal, 1,3-BDO, 4-HBal or 1,4-BDO biosynthetic pathway enzymes or proteins, or enzymes that produce a downstream product related thereto such as an ester or amide thereof. In addition, a non-naturally occurring organism can be generated by mutagenesis of an endogenous gene that results in an increase in activity of an enzyme in the 3-HBal, 1,3-BDO, 4-HBal or 1,4-BDO biosynthetic pathway, or a downstream product related thereto such as an ester or amide thereof.

In some embodiments, provided herein is a non-naturally occurring microbial organism that is a capable of producing more 3-hydroxybutyraldehyde and/or 1,3-butanediol, or an ester or amide thereof compared to a control microbial organism that does not having a recombinant nucleic acid that encodes an engineered aldehyde dehydrogenase described herein. Such a microbial organism, in some embodiments, is capable of producing at least 10% more 3-hydroxybutyraldehyde and/or 1,3-butanediol, or an ester or amide thereof compared to the control microbial organism. In some embodiments, the microbial organism is capable of producing at least 20% more 3-hydroxybutyraldehyde and/or 1,3-butanediol, or an ester or amide thereof compared to the control microbial organism. In some embodiments, the microbial organism is capable of producing at least 30% more 3-hydroxybutyraldehyde and/or 1,3-butanediol, or an ester or amide thereof compared to the control microbial organism. In some embodiments, the microbial organism is capable of producing at least 40% more 3-hydroxybutyraldehyde and/or 1,3-butanediol, or an ester or amide thereof compared to the control microbial organism. In some embodiments, the microbial organism is capable of producing at least 50% more 3-hydroxybutyraldehyde and/or 1,3-butanediol, or an ester or amide thereof compared to the control microbial organism. In some embodiments, the microbial organism is capable of producing at least 60% more 3-hydroxybutyraldehyde and/or 1,3-butanediol, or an ester or amide thereof compared to the control microbial organism. In some embodiments, the microbial organism is capable of producing at least 70% more 3-hydroxybutyraldehyde and/or 1,3-butanediol, or an ester or amide thereof compared to the control microbial organism. In some embodiments, the microbial organism is capable of producing at least 80% more 3-hydroxybutyraldehyde and/or 1,3-butanediol, or an ester or amide thereof compared to the control microbial organism. In some embodiments, the microbial organism is capable of producing at least 90% more 3-hydroxybutyraldehyde and/or 1,3-butanediol, or an ester or amide thereof compared to the control microbial organism. In some embodiments, the microbial organism is capable of producing at least 1 fold more 3-hydroxybutyraldehyde and/or 1,3-butanediol, or an ester or amide thereof compared to the control microbial organism. In some embodiments, the microbial organism is capable of producing at least 1.1 fold more 3-hydroxybutyraldehyde and/or 1,3-butanediol, or an ester or amide thereof compared to the control microbial organism. In some embodiments, the microbial organism is capable of producing at least 1.2 fold more 3-hydroxybutyraldehyde and/or 1,3-butanediol, or an ester or amide thereof compared to the control microbial organism. In some embodiments, the microbial organism is capable of producing at least 1.3 fold more 3-hydroxybutyraldehyde and/or 1,3-butanediol, or an ester or amide thereof compared to the control microbial organism. In some embodiments, the microbial organism is capable of producing at least 1.4 fold more 3-hydroxybutyraldehyde and/or 1,3-butanediol, or an ester or amide thereof compared to the control microbial organism. In some embodiments, the microbial organism is capable of producing at least 1.5 fold more 3-hydroxybutyraldehyde and/or 1,3-butanediol, or an ester or amide thereof compared to the control microbial organism. In some embodiments, the microbial organism is capable of producing at least 1.6 fold more 3-hydroxybutyraldehyde and/or 1,3-butanediol, or an ester or amide thereof compared to the control microbial organism. In some embodiments, the microbial organism is capable of producing at least 1.7 fold more 3-hydroxybutyraldehyde and/or 1,3-butanediol, or an ester or amide thereof compared to the control microbial organism. In some embodiments, the microbial organism is capable of producing at least 1.8 fold more 3-hydroxybutyraldehyde and/or 1,3-butanediol, or an ester or amide thereof compared to the control microbial organism. In some embodiments, the microbial organism is capable of producing at least 1.9 fold more 3-hydroxybutyraldehyde and/or 1,3-butanediol, or an ester or amide thereof compared to the control microbial organism. In some embodiments, the microbial organism is capable of producing at least 2 fold more 3-hydroxybutyraldehyde and/or 1,3-butanediol, or an ester or amide thereof compared to the control microbial organism.

In some embodiments, provided herein is a non-naturally occurring microbial organism that is a capable of producing more 4-hydroxybutyraldehyde and/or 1,4-butanediol, or an ester or amide thereof compared to a control microbial organism that does not having a recombinant nucleic acid that encodes an engineered aldehyde dehydrogenase described herein. Such a microbial organism, in some embodiments, is capable of producing at least 10% more 4-hydroxybutyraldehyde and/or 1,4-butanediol, or an ester or amide thereof compared to the control microbial organism. In some embodiments, the microbial organism is capable of producing at least 20% more 4-hydroxybutyraldehyde and/or 1,4-butanediol, or an ester or amide thereof compared to the control microbial organism. In some embodiments, the microbial organism is capable of producing at least 30% more 4-hydroxybutyraldehyde and/or 1,4-butanediol, or an ester or amide thereof compared to the control microbial organism. In some embodiments, the microbial organism is capable of producing at least 40% more 4-hydroxybutyraldehyde and/or 1,4-butanediol, or an ester or amide thereof compared to the control microbial organism. In some embodiments, the microbial organism is capable of producing at least 50% more 4-hydroxybutyraldehyde and/or 1,4-butanediol, or an ester or amide thereof compared to the control microbial organism. In some embodiments, the microbial organism is capable of producing at least 60% more 4-hydroxybutyraldehyde and/or 1,4-butanediol, or an ester or amide thereof compared to the control microbial organism. In some embodiments, the microbial organism is capable of producing at least 70% more 4-hydroxybutyraldehyde and/or 1,4-butanediol, or an ester or amide thereof compared to the control microbial organism. In some embodiments, the microbial organism is capable of producing at least 80% more 4-hydroxybutyraldehyde and/or 1,4-butanediol, or an ester or amide thereof compared to the control microbial organism. In some embodiments, the microbial organism is capable of producing at least 90% more 4-hydroxybutyraldehyde and/or 1,4-butanediol, or an ester or amide thereof compared to the control microbial organism. In some embodiments, the microbial organism is capable of producing at least 1 fold more 4-hydroxybutyraldehyde and/or 1,4-butanediol, or an ester or amide thereof compared to the control microbial organism. In some embodiments, the microbial organism is capable of producing at least 1.1 fold more 4-hydroxybutyraldehyde and/or 1,4-butanediol, or an ester or amide thereof compared to the control microbial organism. In some embodiments, the microbial organism is capable of producing at least 1.2 fold more 4-hydroxybutyraldehyde and/or 1,4-butanediol, or an ester or amide thereof compared to the control microbial organism. In some embodiments, the microbial organism is capable of producing at least 1.3 fold more 4-hydroxybutyraldehyde and/or 1,4-butanediol, or an ester or amide thereof compared to the control microbial organism. In some embodiments, the microbial organism is capable of producing at least 1.4 fold more 4-hydroxybutyraldehyde and/or 1,4-butanediol, or an ester or amide thereof compared to the control microbial organism. In some embodiments, the microbial organism is capable of producing at least 1.5 fold more 4-hydroxybutyraldehyde and/or 1,4-butanediol, or an ester or amide thereof compared to the control microbial organism. In some embodiments, the microbial organism is capable of producing at least 1.6 fold more 4-hydroxybutyraldehyde and/or 1,4-butanediol, or an ester or amide thereof compared to the control microbial organism. In some embodiments, the microbial organism is capable of producing at least 1.7 fold more 4-hydroxybutyraldehyde and/or 1,4-butanediol, or an ester or amide thereof compared to the control microbial organism. In some embodiments, the microbial organism is capable of producing at least 1.8 fold more 4-hydroxybutyraldehyde and/or 1,4-butanediol, or an ester or amide thereof compared to the control microbial organism. In some embodiments, the microbial organism is capable of producing at least 1.9 fold more 4-hydroxybutyraldehyde and/or 1,4-butanediol, or an ester or amide thereof compared to the control microbial organism. In some embodiments, the microbial organism is capable of producing at least 2 fold more 4-hydroxybutyraldehyde and/or 1,4-butanediol, or an ester or amide thereof compared to the control microbial organism.

In some embodiments, provided herein is a non-naturally occurring microbial organism that produces a decreased amount of a by-product as compared to a control microbial organism that does not having a recombinant nucleic acid that encodes an engineered aldehyde dehydrogenase described herein. By-products that can be decreased by using an engineered aldehyde dehydrogenase provided herein in a non-naturally occurring microbial organism include any product that is not the desired product the engineered aldehyde dehydrogenase is intended to catalyze the production of or a product that represents poor aldehyde dehydrogenase activity. Non-limiting examples of such a by-product include ethanol, 4-hydroxy-2-butanone, 3-hydroxybutyrate or 4-hydroxybutyrate. Accordingly, in some embodiments, the microbial organism provided herein is capable of producing at least 10% less by-product. In some embodiments, the microbial organism provided herein is capable of producing at least 20% less by-product. In some embodiments, the microbial organism provided herein is capable of producing at least 30% less by-product. In some embodiments, the microbial organism provided herein is capable of producing at least 40% less by-product. In some embodiments, the microbial organism provided herein is capable of producing at least 50% less by-product. In some embodiments, the microbial organism provided herein is capable of producing at least 60% less by-product. In some embodiments, the microbial organism provided herein is capable of producing at least 70% less by-product. In some embodiments, the microbial organism provided herein is capable of producing at least 80% less by-product. In some embodiments, the microbial organism provided herein is capable of producing at least 90% less by-product. In some embodiments, the microbial organism provided herein is capable of producing at least 95% less by-product. In some embodiments, the microbial organism provided herein is capable of producing at least 98% less by-product. In some embodiments, the microbial organism provided herein is capable of producing at least 99% less by-product.

In particularly useful embodiments, exogenous expression of the encoding nucleic acids is employed. Exogenous expression confers the ability to custom tailor the expression and/or regulatory elements to the host and application to achieve a desired expression level that is controlled by the user. However, endogenous expression also can be utilized in other embodiments such as by removing a negative regulatory effector or induction of the gene's promoter when linked to an inducible promoter or other regulatory element. Thus, an endogenous gene having a naturally occurring inducible promoter can be up-regulated by providing the appropriate inducing agent, or the regulatory region of an endogenous gene can be engineered to incorporate an inducible regulatory element, thereby allowing the regulation of increased expression of an endogenous gene at a desired time. Similarly, an inducible promoter can be included as a regulatory element for an exogenous gene introduced into a non-naturally occurring cell.

It is understood that, in methods described herein, any of the one or more recombinant and/or exogenous nucleic acids can be introduced into a cell to produce a non-naturally occurring cell provided herein. The nucleic acids can be introduced so as to confer, for example, a 3-HBal, 1,3-BDO, 4-HBal or 1,4-BDO, or a downstream product related thereto such as an ester or amide thereof, biosynthetic pathway onto the cell, including introducing a nucleic acid encoding an engineered aldehyde dehydrogenase provided herein. Alternatively, encoding nucleic acids can be introduced to produce a cell having the biosynthetic capability to catalyze some of the required reactions to confer 3-HBal, 1,3-BDO, 4-HBal or 1,4-BDO biosynthetic capability to produce an intermediate. For example, a non-naturally occurring cell having a 3-HBal, 1,3-BDO, 4-HBal or 1,4-BDO biosynthetic pathway can comprise at least two exogenous nucleic acids encoding desired enzymes or proteins, including an engineered aldehyde dehydrogenase provided herein. Thus, it is understood that any combination of two or more enzymes or proteins of a biosynthetic pathway can be included in a non-naturally occurring cell provided herein, including an engineered aldehyde dehydrogenase provided herein. Similarly, it is understood that any combination of three or more enzymes or proteins of a biosynthetic pathway can be included in a non-naturally occurring cell provided herein, as desired, so long as the combination of enzymes and/or proteins of the desired biosynthetic pathway results in production of the corresponding desired product. Similarly, any combination of five, six, seven, eight, nine, ten, eleven, twelve or more enzymes or proteins of a biosynthetic pathway as disclosed herein can be included in a non-naturally occurring cell provided herein, as desired, so long as the combination of enzymes and/or proteins of the desired biosynthetic pathway results in production of the corresponding desired product.

In addition to the biosynthesis of 3-HBal, 1,3-BDO, 4-HBal or 1,4-BDO, or a downstream product related thereto such as an ester or amide thereof, as described herein, the non-naturally occurring cells and methods provided herein also can be utilized in various combinations with each other and/or with other cells and methods well known in the art to achieve product biosynthesis by other routes. For example, one alternative to produce 3-HBal, 1,3-BDO, 4-HBal or 1,4-BDO other than use of the 3-HBal, 1,3-BDO, 4-HBal or 1,4-BDO producers is through addition of another cell capable of converting a 3-HBal, 1,3-BDO, 4-HBal or 1,4-BDO pathway intermediate to 3-HBal, 1,3-BDO, 4-HBal or 1,4-BDO. One such procedure includes, for example, the fermentation of a cell that produces a 3-HBal, 1,3-BDO, 4-HBal or 1,4-BDO pathway intermediate. The 3-HBal, 1,3-BDO, 4-HBal or 1,4-BDO pathway intermediate can then be used as a substrate for a second cell that converts the 3-HBal, 1,3-BDO, 4-HBal or 1,4-BDO pathway intermediate to 3-HBal, 1,3-BDO, 4-HBal or 1,4-BDO. The 3-HBal, 1,3-BDO, 4-HBal or 1,4-BDO pathway intermediate can be added directly to another culture of the second organism or the original culture of the 3-HBal, 1,3-BDO, 4-HBal or 1,4-BDO pathway intermediate producers can be depleted of these cells by, for example, cell separation, and then subsequent addition of the second organism to the fermentation broth can be utilized to produce the final product without intermediate purification steps. A cell that produces a downstream product related to 3-HBal, 1,3-BDO, 4-HBal or 1,4-BDO such as an ester or amide thereof, can optionally be included to produce such a downstream product.

Alternatively, such enzymatic conversions can be carried out in vitro, with a combination of enzymes or sequential exposure of substrates to enzymes that result in conversion of a substrate to a desired product. As another alternative, a combination of cell-based conversions and in vitro enzymatic conversions can be used, if desired.

In other embodiments, the non-naturally occurring cells and methods provided herein can be assembled in a wide variety of subpathways to achieve biosynthesis of, for example, 3-HBal, 1,3-BDO, 4-HBal or 1,4-BDO or a downstream product related thereto such as an ester or amide thereof. In these embodiments, biosynthetic pathways for a desired product provided herein can be segregated into different cells, and the different cells can be co-cultured to produce the final product. In such a biosynthetic scheme, the product of one cell is the substrate for a second cell until the final product is synthesized. For example, the biosynthesis of 3-HBal, 1,3-BDO, 4-HBal or 1,4-BDO, or a downstream product related thereto such as an ester or amide thereof, can be accomplished by constructing a cell that contains biosynthetic pathways for conversion of one pathway intermediate to another pathway intermediate or the product. Alternatively, 3-HBal, 1,3-BDO, 4-HBal or 1,4-BDO also can be biosynthetically produced from cells through co-culture or co-fermentation using two different cells in the same vessel, where the first cell produces a 3-HBal, 1,3-BDO, 4-HBal or 1,4-BDO intermediate and the second cell converts the intermediate to 3-HBal, 1,3-BDO, 4-HBal or 1,4-BDO, or a downstream product related thereto such as an ester or amide thereof.

Given the teachings and guidance provided herein, those skilled in the art will understand that a wide variety of combinations and permutations exist for the non-naturally occurring cells and methods provided herein together with other cells, with the co-culture of other non-naturally occurring cells having subpathways and with combinations of other chemical and/or biochemical procedures well known in the art to produce 3-HBal, 1,3-BDO, 4-HBal or 1,4-BDO, or a downstream product related thereto such as an ester or amide thereof.

Similarly, it is understood by those skilled in the art that a host organism can be selected based on desired characteristics for introduction of one or more gene disruptions to increase synthesis or production of 3-HBal, 1,3-BDO, 4-HBal or 1,4-BDO, or a downstream product related thereto such as an ester or amide thereof. Thus, it is understood that, if a genetic modification is to be introduced into a host organism to disrupt a gene, any homologs, orthologs or paralogs that catalyze similar, yet non-identical metabolic reactions can similarly be disrupted to ensure that a desired metabolic reaction is sufficiently disrupted. Because certain differences exist among metabolic networks between different organisms, those skilled in the art will understand that the actual genes disrupted in a given organism may differ between organisms. However, given the teachings and guidance provided herein, those skilled in the art also will understand that the methods described herein can be applied to any suitable host microorganism to identify the cognate metabolic alterations needed to construct an organism in a species of interest that will increase biosynthesis of 3-HBal, 1,3-BDO, 4-HBal or 1,4-BDO, or a downstream product related thereto such as an ester or amide thereof. In a particular embodiment, the increased production couples biosynthesis of 3-HBal, 1,3-BDO, 4-HBal or 1,4-BDO, or a downstream product related thereto such as an ester or amide thereof to growth of the organism, and can obligatorily couple production of 3-HBal, 1,3-BDO, 4-HBal or 1,4-BDO, or a downstream product related thereto such as an ester or amide thereof to growth of the organism if desired and as disclosed herein.

Sources of encoding nucleic acids for a 3-HBal, 1,3-BDO, 4-HBal or 1,4-BDO pathway enzyme or protein, or a downstream product related thereto such as an ester or amide thereof, can include, for example, any species where the encoded gene product is capable of catalyzing the referenced reaction. Such species include both prokaryotic and eukaryotic organisms including, but not limited to, bacteria, including archaea and eubacteria, and eukaryotes, including yeast, plant, insect, animal, and mammal, including human. Exemplary species for such sources include, for example, Escherichia coli, Saccharomyces cerevisiae, Saccharomyces kluyveri, Clostridium kluyveri, Clostridium acetobutylicum, Clostridium beijerinckii, Clostridium saccharoperbutylacetonicum, Clostridium perfringens, Clostridium difficile, Clostridium botulinum, Clostridium tyrobutyricum, Clostridium tetanomorphum, Clostridium tetani, Clostridium propionicum, Clostridium aminobutyricum, Clostridium subterminale, Clostridium sticklandii, Ralstonia eutropha, Mycobacterium bovis, Mycobacterium tuberculosis, Porphyromonas gingivalis, Arabidopsis thaliana, Thermus thermophilus, Pseudomonas species, including Pseudomonas aeruginosa, Pseudomonas putida, Pseudomonas stutzeri, Pseudomonas fluorescens, Homo sapiens, Oryctolagus cuniculus, Rhodobacter spaeroides, Thermoanaerobacter brockii, Metallosphaera sedula, Leuconostoc mesenteroides, Chloroflexus aurantiacus, Roseiflexus castenholzii, Erythrobacter, Simmondsia chinensis, Acinetobacter species, including Acinetobacter calcoaceticus and Acinetobacter baylyi, Porphyromonas gingivalis, Sulfolobus tokodaii, Sulfolobus solfataricus, Sulfolobus acidocaldarius, Bacillus subtilis, Bacillus cereus, Bacillus megaterium, Bacillus brevis, Bacillus pumilus, Rattus norvegicus, Klebsiella pneumonia, Klebsiella oxytoca, Euglena gracilis, Treponema denticola, Moorella thermoacetica, Thermotoga maritima, Halobacterium salinarum, Geobacillus stearothermophilus, Aeropyrum pernix, Sus scrofa, Caenorhabditis elegans, Corynebacterium glutamicum, Acidaminococcus fermentans, Lactococcus lactis, Lactobacillus plantarum, Streptococcus thermophilus, Enterobacter aerogenes, Candida, Aspergillus terreus, Pedicoccus pentosaceus, Zymomonas mobilus, Acetobacter pasteurians, Kluyveromyces lactis, Eubacterium barkeri, Bacteroides capillosus, Anaerotruncus colihominis, Natranaerobius thermophilusm, Campylobacter jejuni, Haemophilus influenzae, Serratia marcescens, Citrobacter amalonaticus, Myxococcus xanthus, Fusobacterium nuleatum, Penicillium chrysogenum, marine gamma proteobacterium, butyrate-producing bacterium, Nocardia iowensis, Nocardia farcinica, Streptomyces griseus, Schizosaccharomyces pombe, Geobacillus thermoglucosidasius, Salmonella typhimurium, Vibrio cholera, Heliobacter pylori, Nicotiana tabacum, Oryza sativa, Haloferax mediterranei, Agrobacterium tumefaciens, Achromobacter denitrificans, Fusobacterium nucleatum, Streptomyces clavuligenus, Acinetobacter baumanii, Mus musculus, Lachancea kluyveri, Trichomonas vaginalis, Trypanosoma brucei, Pseudomonas stutzeri, Bradyrhizobium japonicum, Mesorhizobium loti, Bos taurus, Nicotiana glutinosa, Vibrio vulniicus, Selenomonas ruminantium, Vibrio parahaemolyticus, Archaeoglobus fulgidus, Haloarcula marismortui, Pyrobaculum aerophilum, Mycobacterium smegmatis MC2 155, Mycobacterium avium subsp. paratuberculosis K-10, Mycobacterium marinum M, Tsukamurella paurometabola DSM 20162, Cyanobium PCC7001, Dictyostelium discoideum AX4, Acidaminococcus fermentans, Acinetobacter baylyi, Acinetobacter calcoaceticus, Aquifex aeolicus, Arabidopsis thaliana, Archaeoglobus fulgidus, Aspergillus niger, Aspergillus terreus, Bacillus subtilis, Bos Taurus, Candida albicans, Candida tropicalis, Chlamydomonas reinhardtii, Chlorobium tepidum, Citrobacter koseri, Citrus junos, Clostridium acetobutylicum, Clostridium kluyveri, Clostridium saccharoperbutylacetonicum, Cyanobium PCC7001, Desulfati bacillum alkenivorans, Dictyostelium discoideum, Fusobacterium nucleatum, Haloarcula marismortui, Homo sapiens, Hydrogenobacter thermophilus, Klebsiella pneumoniae, Kluyveromyces lactis, Lactobacillus brevis, Leuconostoc mesenteroides, Metallosphaera sedula, Methanothermobacter thermautotrophicus, Mus musculus, Mycobacterium avium, Mycobacterium bovis, Mycobacterium marinum, Mycobacterium smegmatis, Nicotiana tabacum, Nocardia iowensis, Oryctolagus cuniculus, Penicillium chrysogenum, Pichia pastoris, Porphyromonas gingivalis, Porphyromonas gingivalis, Pseudomonas aeruginos, Pseudomonas putida, Pyrobaculum aerophilum, Ralstonia eutropha, Rattus norvegicus, Rhodobacter sphaeroides, Saccharomyces cerevisiae, Salmonella enteric, Salmonella typhimurium, Schizosaccharomyces pombe, Sulfolobus acidocaldarius, Sulfolobus solfataricus, Sulfolobus tokodaii, Thermoanaerobacter tengcongensis, Thermus thermophilus, Trypanosoma brucei, Tsukamurella paurometabola, Yarrowia lipolytica, Zoogloea ramigera and Zymomonas mobilis, Clostridum species, including but no limited to Clostridium saccharoperbutylacetonicum, Clostridium beijerinckii, Clostridium saccharobutylicum, Clostridium botulinum, Clostridium methylpentosum, Clostridium sticklandii, Clostridium phytofermentans, Clostridium saccharolyticum, Clostridium asparagiforme, Clostridium celatum, Clostridium carboxidivorans, Clostridium clostridioforme, Clostridium bolteae, Caldalkalibacillus thermarum, Clostridium botulinum, Pelosinus fermentans, Thermoanaerobacterium thermosaccharolyticum, Desulfosporosinus speices, Thermoanaerobacterium species, including but not limited to Thermoanaerobacterium saccharolyticum, Thermoanaerobacterium xylanolyticum, Acetonema longum, Geobacillus species, including but not limited to Geobacillus thermoglucosidans, Bacillus azotoformans, Thermincola potens, Fusobacterium species, including but not limited to Fusobacterium nucleatum, Fusobacterium ulcerans, Fusobacterium varium, Ruminococcus species, including but not limited to Ruminococcus gnavus, Ruminococcus obeum, Lachnospiraceae bacterium, Flavonifractor plautii, Roseburia inulinivorans, Acetobacterium woodii, Eubacterium species, including but not limited to Eubacterium plexicaudatum, Eubacterium hallii, Eubacterium limosum, Eubacteriuml yurii, Eubacteriaceae bacterium, Thermosediminibacter oceani, Ilyobacter polytropus, Shuttleworthia satelles, Halanaerobium saccharolyticum, Thermoanaerobacter ethanolicus, Rhodospirillum rubrum, Vibrio, Propionibacterium propionicum as well as other exemplary species disclosed herein or available as source organisms for corresponding genes. However, with the complete genome sequence available for now more than 550 species (with more than half of these available on public databases such as the NCBI), including 395 microorganism genomes and a variety of yeast, fungi, plant, and mammalian genomes, the identification of genes encoding the 3-HBal, 1,3-BDO, 4-HBal or 1,4-BDO biosynthetic activity for one or more genes in related or distant species, including for example, homologues, orthologs, paralogs and nonorthologous gene displacements of known genes, and the interchange of genetic alterations between organisms is routine and well known in the art. Accordingly, the metabolic alterations allowing biosynthesis of 3-HBal, 1,3-BDO, 4-HBal or 1,4-BDO, or a downstream product related thereto such as an ester or amide thereof, including expression of an engineered aldehyde dehydrogenase provided herein, described herein with reference to a particular organism such as E. coli can be readily applied to other cells such as microorganisms, including prokaryotic and eukaryotic organisms alike. Given the teachings and guidance provided herein, those skilled in the art will know that a metabolic alteration exemplified in one organism can be applied equally to other organisms.

In some instances, such as when an alternative 3-HBal, 1,3-BDO, 4-HBal or 1,4-BDO biosynthetic pathway exists in an unrelated species, 3-HBal, 1,3-BDO, 4-HBal or 1,4-BDO biosynthesis can be conferred onto the host species by, for example, exogenous expression of a paralog or paralogs from the unrelated species that catalyzes a similar, yet non-identical metabolic reaction to replace the referenced reaction. Because certain differences among metabolic networks exist between different organisms, those skilled in the art will understand that the actual gene usage between different organisms may differ. However, given the teachings and guidance provided herein, those skilled in the art also will understand that the teachings and methods provided herein can be applied to all cells using the cognate metabolic alterations to those exemplified herein to construct a cell in a species of interest that will synthesize 3-HBal, 1,3-BDO, 4-HBal or 1,4-BDO, or a downstream product related thereto such as an ester or amide thereof, if desired, including introducing an engineered aldehyde dehydrogenase provided herein.

Methods for constructing and testing the expression levels of a non-naturally occurring host producing 3-HBal, 1,3-BDO, 4-HBal or 1,4-BDO, or a downstream product related thereto such as an ester or amide thereof, including an engineered aldehyde dehydrogenase provided herein, can be performed, for example, by recombinant and detection methods well known in the art. Such methods can be found described in, for example, Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, MD (1999).

A recombinant nucleic acid encoding an engineered aldehyde dehydrogenase provided herein, and optionally exogenous nucleic acid sequences involved in a pathway for production of 3-HBal, 1,3-BDO, 4-HBal or 1,4-BDO, or a downstream product related thereto such as an ester or amide thereof, can be introduced stably or transiently into a host cell using techniques well known in the art including, but not limited to, conjugation, electroporation, chemical transformation, transduction, transfection, and ultrasound transformation. For exogenous expression in E. coli or other prokaryotic cells, some nucleic acid sequences in the genes or cDNAs of eukaryotic nucleic acids can encode targeting signals such as an N-terminal mitochondrial or other targeting signal, which can be removed before transformation into prokaryotic host cells, if desired. For example, removal of a mitochondrial leader sequence led to increased expression in E. coli (Hoffmeister et al., J. Biol. Chem. 280:4329-4338 (2005)). For exogenous expression in yeast or other eukaryotic cells, genes can be expressed in the cytosol without the addition of leader sequence, or can be targeted to mitochondrion or other organelles, or targeted for secretion, by the addition of a suitable targeting sequence such as a mitochondrial targeting or secretion signal suitable for the host cells. Thus, it is understood that appropriate modifications to a nucleic acid sequence to remove or include a targeting sequence can be incorporated into an exogenous nucleic acid sequence to impart desirable properties. Furthermore, genes can be subjected to codon optimization with techniques well known in the art to achieve optimized expression of the proteins.

An expression vector or vectors can be constructed to include a recombinant nucleic acid encoding an engineered aldehyde dehydrogenase provided herein, and/or optionally and/or an exogenous nucleic acid encoding a 3-HBal, 1,3-BDO, 4-HBal or 1,4-BDO biosynthetic pathway, or nucleic acids encoding an enzyme that produces a downstream product related to 3-HBal, 1,3-BDO, 4-HBal or 1,4-BDO such as an ester or amide thereof, as exemplified herein operably linked to expression control sequences functional in the host organism. Expression vectors applicable for use in the host cells provided herein include, for example, plasmids, phage vectors, viral vectors, episomes and artificial chromosomes, including vectors and selection sequences or markers operable for stable integration into a host chromosome. Additionally, the expression vectors can include one or more selectable marker genes and appropriate expression control sequences. Selectable marker genes also can be included that, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more exogenous encoding nucleic acids are to be co-expressed, both nucleic acids can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The transformation of exogenous nucleic acid sequences encoding an engineered aldehyde dehydrogenase provided herein or encoding polypeptides involved in a metabolic or synthetic pathway can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the exogenous nucleic acid is expressed in a sufficient amount to produce the desired product, and it is further understood that expression levels can be optimized to obtain sufficient expression using methods well known in the art and as disclosed herein.

A vector or expression vector can also be used to express an encoded nucleic acid to produce an encoded polypeptide by in vitro transcription and translation. Such a vector or expression vector will comprise at least a promoter, and includes the vectors described herein above. Such a vector for in vitro transcription and translation generally is double stranded DNA. Methods of in vitro transcription and translation are well known to those skilled in the art (see Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, MD (1999)). Kits for in vitro transcription and translation are also commercially available (see, for example, Promega, Madison, WI; New England Biolabs, Ipswich, MA; Thermo Fisher Scientific, Carlsbad, CA).

In some embodiments, provided herein is a non-naturally occurring microbial organism having a vector described herein comprising a nucleic acid described herein. Also provided a non-naturally occurring microbial organism having a nucleic acid described herein. In some embodiments, the nucleic acid is integrated into a chromosome of the organism. In some embodiments, the integration is site-specific. In an embodiment described herein, the nucleic acid is expressed. In some embodiments, provided herein is a non-naturally occurring microbial organism having an engineered aldehyde dehydrogenase described herein.

In some embodiments, provided herein is a method for producing 3-hydroxybutyraldehyde (3-HBal) and/or 1,3-butanediol (1,3-BDO), or an ester or amide thereof, comprising culturing a cell provided herein under conditions and for a sufficient period of time to produce 3-HBal and/or 1,3-BDO, or an ester or amide thereof. Such a cell expresses an engineered aldehyde dehydrogenase provided herein. In some embodiments, provided herein is a method for producing 4-hydroxybutyraldehyde (4-HBal) and/or 1,4-butanediol (1,4-BDO), or an ester or amide thereof, comprising culturing a cell provided herein under conditions and for a sufficient period of time to produce 4-HBal and/or 1,4-BDO, or an ester or amide thereof. In one embodiment, the cell is in a substantially anaerobic culture medium. In some embodiments, the method can further comprise isolating or purifying the 3-HBal and/or 1,3-BDO, or the 4-HBal and/or 1,4-BDO, or ester or amide thereof. In a particular embodiment, the isolating or purifying comprises distillation.

In one embodiment, provided herein a process for producing a product provided herein, comprising chemically reacting the 3-HBal and/or 1,3-BDO, or the 4-HBal and/or 1,4-BDO, with itself or another compound in a reaction that produces the product.

In one embodiment, provided herein is a method for producing 3-hydroxybutyraldehyde (3-HBal) and/or 1,3-butanediol (1,3-BDO), or an ester or amide thereof, comprising providing a substrate to an engineered aldehyde dehydrogenase provided herein and converting the substrate to 3-HBal and/or 1,3-BDO, wherein the substrate is a racemic mixture of 1,3-hydroxybutyryl-CoA. In one embodiment, the 3-HBal and/or 1,3-BDO is enantiomerically enriched for the R form. In one embodiment, provided herein is a method for producing 4-hydroxybutyraldehyde (4-HBal) and/or 1,4-butanediol (1,4-BDO), or an ester or amide thereof, comprising providing a substrate to an engineered aldehyde dehydrogenase provided herein and converting the substrate to 4-HBal and/or 1,4-BDO, wherein the substrate is 1,4-hydroxybutyryl-CoA. In one embodiment, the engineered aldehyde dehydrogenase is present in a cell, in a cell lysate, or is isolated from a cell or cell lysate.

In one embodiment, provided herein is a method for producing 3-HBal and/or 1,3-BDO, or 4-HBal and/or 1,4-BDO, comprising incubating a lysate of a cell provided herein to produce 3-HBal and/or 1,3-BDO, or 4-HBal and/or 1,4-BDO. In one embodiment, the cell lysate is mixed with a second cell lysate, wherein the second cell lysate comprises an enzymatic activity to produce a substrate of an engineered aldehyde dehydrogenase provided herein, or a downstream product of 3-HBal and/or 1,3-BDO, or 4-HBal and/or 1,4-BDO.

Also provided herein is a method for producing an engineered aldehyde dehydrogenase provided herein, comprising expressing the engineered aldehyde dehydrogenase in a cell. Still further provided herein is a method for producing an engineered aldehyde dehydrogenase provided herein, comprising in vitro transcribing and translating a nucleic acid provided herein or a vector provided herein to produce the engineered aldehyde dehydrogenase.

As described herein, a cell can be used to express an engineered aldehyde dehydrogenase provided herein, and optionally the cell can include a metabolic pathway that utilizes an engineered aldehyde dehydrogenase provided herein to produce a desired product, such as 3-HBal and/or 1,3-BDO, or 4-HBal and/or 1,4-BDO. Such methods for expressing a desired product are described herein. Alternatively, an engineered aldehyde dehydrogenase provided herein can be expressed, and/or a desired product produced, in a cell lysate, for example, a cell lysate of a cell expressing an engineered aldehyde dehydrogenase provided herein, or a cell expressing an engineered aldehyde dehydrogenase provided herein and a metabolic pathway to produce a desired product, as described herein. In another embodiment, an engineered aldehyde dehydrogenase provided herein can be expressed by in vitro transcription and translation, in which the aldehyde dehydrogenase is produced in a cell free system. The aldehyde dehydrogenase expressed by in vitro transcription and translation can be used to carry out a reaction in vitro. Optionally, other enzymes, or cell lysate(s) containing such enzymes, can be used to convert the product of the aldehyde dehydrogenase enzymatic reaction to a desired downstream product in vitro.

Suitable purification and/or assays to test for the expression of an aldehyde dehydrogenase, or for production of 3-HBal, 1,3-BDO, 4-HBal or 1,4-BDO, or a downstream product related thereto such as an ester or amide thereof, including assays to test for aldehyde dehydrogenase activity, can be performed using well known methods (see also Example). Suitable replicates such as triplicate cultures can be grown for each engineered strain to be tested. For example, product and byproduct formation in the engineered production host can be monitored. The final product and intermediates, and other organic compounds, can be analyzed by methods such as HPLC (High Performance Liquid Chromatography), GC-MS (Gas Chromatography-Mass Spectroscopy) and LC-MS (Liquid Chromatography-Mass Spectroscopy) or other suitable analytical methods using routine procedures well known in the art. The release of product in the fermentation broth can also be tested with the culture supernatant. Byproducts and residual glucose can be quantified by HPLC using, for example, a refractive index detector for glucose and alcohols, and a UV detector for organic acids (Lin et al., Biotechnol. Bioeng. 90:775-779 (2005)), or other suitable assay and detection methods well known in the art. The individual enzyme or protein activities from the exogenous DNA sequences can also be assayed using methods well known in the art (see also Example).

The 3-HBal, 1,3-BDO, 4-HBal or 1,4-BDO, or other desired product, such as a downstream product related thereto such as an ester or amide thereof, can be separated from other components in the culture using a variety of methods well known in the art. Such separation methods include, for example, extraction procedures as well as methods that include continuous liquid-liquid extraction, pervaporation, membrane filtration, membrane separation, reverse osmosis, electrodialysis, distillation, crystallization, centrifugation, extractive filtration, ion exchange chromatography, size exclusion chromatography, adsorption chromatography, or ultrafiltration. All of the above methods are well known in the art.

Any of the non-naturally occurring cells expressing an engineered aldehyde dehydrogenase provided herein described herein can be cultured to produce and/or secrete the biosynthetic products provided herein. For example, the cells that produce 3-HBal, 1,3-BDO, 4-HBal or 1,4-BDO, or a downstream product related thereto such as an ester or amide thereof, can be cultured for the biosynthetic production of 3-HBal, 1,3-BDO, 4-HBal or 1,4-BDO, or a downstream product related thereto such as an ester or amide thereof. Accordingly, in some embodiments, provided herein is a culture medium containing the 3-HBal, 1,3-BDO, 4-HBal or 1,4-BDO, or a downstream product related thereto such as an ester or amide thereof, or 3-HBal, 1,3-BDO, 4-HBal or 1,4-BDO pathway intermediate described herein. In some aspects, the culture medium can also be separated from the non-naturally occurring cells provided herein that produced the 3-HBal, 1,3-BDO, 4-HBal or 1,4-BDO, or a downstream product related thereto such as an ester or amide thereof, or 3-HBal, 1,3-BDO, 4-HBal or 1,4-BDO pathway intermediate. Methods for separating a cell from culture medium are well known in the art. Exemplary methods include filtration, flocculation, precipitation, centrifugation, sedimentation, and the like.

For the production of an engineered aldehyde dehydrogenase provided herein, or of 3-HBal, 1,3-BDO, 4-HBal or 1,4-BDO, or a downstream product related thereto such as an ester or amide thereof, in a cell expressing an engineered aldehyde dehydrogenase provided herein, the recombinant strains are cultured in a medium with carbon source and other essential nutrients. It is sometimes desirable and can be highly desirable to maintain anaerobic conditions in the fermenter to reduce the cost of the overall process. Such conditions can be obtained, for example, by first sparging the medium with nitrogen and then sealing the flasks with a septum and crimp-cap. For strains where growth is not observed anaerobically, microaerobic or substantially anaerobic conditions can be applied by perforating the septum with a small hole for limited aeration. Exemplary anaerobic conditions have been described previously and are well-known in the art. Exemplary aerobic and anaerobic conditions are described, for example, in United States publication 2009/0047719, filed Aug. 10, 2007. Fermentations can be performed in a batch, fed-batch or continuous manner, as disclosed herein. Fermentations can also be conducted in two phases, if desired. The first phase can be aerobic to allow for high growth and therefore high productivity, followed by an anaerobic phase of high yields of a desired product such as 3-HBal, 1,3-BDO, 4-HBal or 1,4-BDO, or a downstream product related thereto such as an ester or amide thereof.

If desired, the pH of the medium can be maintained at a desired pH, in particular neutral pH, such as a pH of around 7 by addition of a base, such as NaOH or other bases, or acid, as needed to maintain the culture medium at a desirable pH. The growth rate can be determined by measuring optical density using a spectrophotometer (600 nm), and the glucose uptake rate by monitoring carbon source depletion over time.

The growth medium can include, for example, any carbohydrate source which can supply a source of carbon to the non-naturally occurring cell. Such sources include, for example: sugars such as glucose, xylose, arabinose, galactose, mannose, fructose, sucrose and starch; or glycerol, and it is understood that a carbon source can be used alone as the sole source of carbon or in combination with other carbon sources described herein or known in the art. In one embodiment, the carbon source is a sugar. In one embodiment, the carbon source is a sugar-containing biomass. In some embodiments, the sugar is glucose. In one embodiment, the sugar is xylose. In another embodiment, the sugar is arabinose. In one embodiment, the sugar is galactose. In another embodiment, the sugar is fructose. In other embodiments, the sugar is sucrose. In one embodiment, the sugar is starch. In certain embodiments, the carbon source is glycerol. In some embodiments, the carbon source is crude glycerol. In one embodiment, the carbon source is crude glycerol without treatment. In other embodiments, the carbon source is glycerol and glucose. In another embodiment, the carbon source is methanol and glycerol. In one embodiment, the carbon source is carbon dioxide. In one embodiment, the carbon source is formate. In one embodiment, the carbon source is methane. In one embodiment, the carbon source is methanol. In certain embodiments, methanol is used alone as the sole source of carbon or in combination with other carbon sources described herein or known in the art. In a specific embodiment, the methanol is the only (sole) carbon source. In one embodiment, the carbon source is chemoelectro-generated carbon (see, e.g., Liao et al. (2012) Science 335:1596). In one embodiment, the chemoelectro-generated carbon is methanol. In one embodiment, the chemoelectro-generated carbon is formate. In one embodiment, the chemoelectro-generated carbon is formate and methanol. In one embodiment, the carbon source is a carbohydrate and methanol. In one embodiment, the carbon source is a sugar and methanol. In another embodiment, the carbon source is a sugar and glycerol. In other embodiments, the carbon source is a sugar and crude glycerol. In yet other embodiments, the carbon source is a sugar and crude glycerol without treatment. In one embodiment, the carbon source is a sugar-containing biomass and methanol. In another embodiment, the carbon source is a sugar-containing biomass and glycerol. In other embodiments, the carbon source is a sugar-containing biomass and crude glycerol. In yet other embodiments, the carbon source is a sugar-containing biomass and crude glycerol without treatment. In some embodiments, the carbon source is a sugar-containing biomass, methanol and a carbohydrate. Other sources of carbohydrate include, for example, renewable feedstocks and biomass. Exemplary types of biomasses that can be used as feedstocks in the methods provided herein include cellulosic biomass, hemicellulosic biomass and lignin feedstocks or portions of feedstocks. Such biomass feedstocks contain, for example, carbohydrate substrates useful as carbon sources such as glucose, xylose, arabinose, galactose, mannose, fructose and starch. Given the teachings and guidance provided herein, those skilled in the art will understand that renewable feedstocks and biomass other than those exemplified above also can be used for culturing the cells provided herein for the expression of an engineered aldehyde dehydrogenase provided herein, and optionally production of 3-HBal, 1,3-BDO, 4-HBal or 1,4-BDO, or a downstream product thereof, such as an ester or amide thereof.

In addition to renewable feedstocks such as those exemplified above, the cells provided herein that produce 3-HBal, 1,3-BDO, 4-HBal or 1,4-BDO or a downstream product thereof, such as an ester or amide thereof, also can be modified for growth on syngas as its source of carbon. In this specific embodiment, one or more proteins or enzymes are expressed in the 3-HBal, 1,3-BDO, 4-HBal or 1,4-BDO producing organisms to provide a metabolic pathway for utilization of syngas or other gaseous carbon source.

Synthesis gas, also known as syngas or producer gas, is the major product of gasification of coal and of carbonaceous materials such as biomass materials, including agricultural crops and residues. Syngas is a mixture primarily of H₂ and CO and can be obtained from the gasification of any organic feedstock, including but not limited to coal, coal oil, natural gas, biomass, and waste organic matter. Gasification is generally carried out under a high fuel to oxygen ratio. Although largely H₂ and CO, syngas can also include CO₂ and other gases in smaller quantities. Thus, synthesis gas provides a cost effective source of gaseous carbon such as CO and, additionally, CO₂.

The Wood-Ljungdahl pathway catalyzes the conversion of CO and H₂ to acetyl-CoA and other products such as acetate. Organisms capable of utilizing CO and syngas also generally have the capability of utilizing CO₂ and CO₂/H₂ mixtures through the same basic set of enzymes and transformations encompassed by the Wood-Ljungdahl pathway. H₂-dependent conversion of CO₂ to acetate by microorganisms was recognized long before it was revealed that CO also could be used by the same organisms and that the same pathways were involved. Many acetogens have been shown to grow in the presence of CO₂ and produce compounds such as acetate as long as hydrogen is present to supply the necessary reducing equivalents (see for example, Drake, Acetogenesis, pp. 3-60 Chapman and Hall, New York, (1994)). This can be summarized by the following equation:

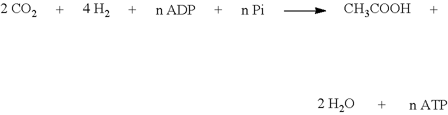

Hence, non-naturally occurring microorganisms possessing the Wood-Ljungdahl pathway can utilize CO₂ and H₂ mixtures as well for the production of acetyl-CoA and other desired products.

The Wood-Ljungdahl pathway is well known in the art and consists of 12 reactions which can be separated into two branches: (1) methyl branch and (2) carbonyl branch. The methyl branch converts syngas to methyl-tetrahydrofolate (methyl-THF) whereas the carbonyl branch converts methyl-TiF to acetyl-CoA. The reactions in the methyl branch are catalyzed in order by the following enzymes or proteins: ferredoxin oxidoreductase, formate dehydrogenase, formyltetrahydrofolate synthetase, methenyltetrahydrofolate cyclodehydratase, methylenetetrahydrofolate dehydrogenase and methylenetetrahydrofolate reductase. The reactions in the carbonyl branch are catalyzed in order by the following enzymes or proteins: methyltetrahydrofolate:corrinoid protein methyltransferase (for example, AcsE), corrinoid iron-sulfur protein, nickel-protein assembly protein (for example, AcsF), ferredoxin, acetyl-CoA synthase, carbon monoxide dehydrogenase and nickel-protein assembly protein (for example, CooC)(see WO2009/094485). Following the teachings and guidance provided herein for introducing a sufficient number of encoding nucleic acids to generate a 3-HBal, 1,3-BDO, 4-HBal or 1,4-BDO pathway, or a downstream product related thereto such as an ester or amide thereof, including a nucleic acid encoding an engineered aldehyde dehydrogenase provided herein, those skilled in the art will understand that the same engineering design also can be performed with respect to introducing at least the nucleic acids encoding the Wood-Ljungdahl enzymes or proteins absent in the host organism. Therefore, introduction of one or more encoding nucleic acids into the cells provided herein such that the modified organism contains the complete Wood-Ljungdahl pathway will confer syngas utilization ability.

Additionally, the reductive (reverse) tricarboxylic acid cycle coupled with carbon monoxide dehydrogenase and/or hydrogenase activities can also be used for the conversion of CO, CO₂ and/or H₂ to acetyl-CoA and other products such as acetate. Organisms capable of fixing carbon via the reductive TCA pathway can utilize one or more of the following enzymes: ATP citrate-lyase, citrate lyase, aconitase, isocitrate dehydrogenase, alpha-ketoglutarate:ferredoxin oxidoreductase, succinyl-CoA synthetase, succinyl-CoA transferase, fumarate reductase, fumarase, malate dehydrogenase, NAD(P)H:ferredoxin oxidoreductase, carbon monoxide dehydrogenase, and hydrogenase. Specifically, the reducing equivalents extracted from CO and/or H₂ by carbon monoxide dehydrogenase and hydrogenase are utilized to fix CO₂ via the reductive TCA cycle into acetyl-CoA or acetate. Acetate can be converted to acetyl-CoA by enzymes such as acetyl-CoA transferase, acetate kinase/phosphotransacetylase, and acetyl-CoA synthetase. Acetyl-CoA can be converted to glyceraldehyde-3-phosphate, phosphoenolpyruvate, and pyruvate, by pyruvate:ferredoxin oxidoreductase and the enzymes of gluconeogenesis. Acetyl-CoA can also be converted to acetoacetyl-CoA by, for example, acetoacetyl-CoA thiolase to funnel into a 1,3-BDO pathway, as disclosed herein (see FIG. 1). Following the teachings and guidance provided herein for introducing a sufficient number of encoding nucleic acids to generate a 3-HBal, 1,3-BDO, 4-HBal or 1,4-BDO pathway, or pathway to generate a downstream product related thereto such as an ester or amide thereof, those skilled in the art will understand that the same engineering design also can be performed with respect to introducing at least the nucleic acids encoding the reductive TCA pathway enzymes or proteins absent in the host organism. Therefore, introduction of one or more encoding nucleic acids into the cells provided herein can be performed such that the modified organism contains a reductive TCA pathway.

Accordingly, given the teachings and guidance provided herein, those skilled in the art will understand that a non-naturally occurring cell can be produced that produces and/or secretes the biosynthesized compounds provided herein when grown on a carbon source such as a carbohydrate. Such compounds include, for example, 3-HBal, 1,3-BDO, 4-HBal or 1,4-BDO, or a downstream product related thereto such as an ester or amide thereof, and any of the intermediate metabolites in the 3-HBal, 1,3-BDO, 4-HBal or 1,4-BDO pathway. All that is required is to engineer in one or more of the required enzyme or protein activities to achieve biosynthesis of the desired compound or intermediate including, for example, inclusion of some or all of the biosynthetic pathways for 3-HBal, 1,3-BDO, 4-HBal or 1,4-BDO, or a downstream product related thereto such as an ester or amide thereof, including an engineered aldehyde dehydrogenase provided herein. Accordingly, provided herein is a non-naturally occurring cell that produces and/or secretes 3-HBal, 1,3-BDO, 4-HBal or 1,4-BDO, or a downstream product related thereto such as an ester or amide thereof, when grown on a carbohydrate or other carbon source and produces and/or secretes any of the intermediate metabolites shown in the 3-HBal, 1,3-BDO, 4-HBal or 1,4-BDO pathway when grown on a carbohydrate or other carbon source. The cells producing 3-HBal, 1,3-BDO, 4-HBal or 1,4-BDO, or a downstream product related thereto such as an ester or amide thereof, provided herein can initiate synthesis from an intermediate of a 3-HBal, 1,3-BDO, 4-HBal or 1,4-BDO pathway.

The non-naturally occurring cells provided herein are constructed using methods well known in the art as exemplified herein to exogenously express an engineered aldehyde dehydrogenase provided herein, and optionally at least one nucleic acid encoding a 3-HBal, 1,3-BDO, 4-HBal or 1,4-BDO pathway enzyme or protein, or a downstream product related thereto such as an ester or amide thereof. The enzymes or proteins can be expressed in sufficient amounts to produce 3-HBal, 1,3-BDO, 4-HBal or 1,4-BDO, or a downstream product related thereto such as an ester or amide thereof. It is understood that the cells provided herein are cultured under conditions sufficient to express an engineered aldehyde dehydrogenase provided herein or produce 3-HBal, 1,3-BDO, 4-HBal or 1,4-BDO, or a downstream product related thereto such as an ester or amide thereof. Following the teachings and guidance provided herein, the non-naturally occurring cells provided herein can achieve biosynthesis of 3-HBal, 1,3-BDO, 4-HBal or 1,4-BDO, or a downstream product related thereto such as an ester or amide thereof, resulting in intracellular concentrations between about 0.1-300 mM or more, for example, 0.1-1.3 M or higher. Generally, the intracellular concentration of 3-HBal, 1,3-BDO, 4-HBal or 1,4-BDO, or a downstream product related thereto such as an ester or amide thereof, is between about 3-150 mM, particularly between about 5-125 mM and more particularly between about 8-100 mM, including about 10 mM, 20 mM, 50 mM, 80 mM, or more. Intracellular concentrations between and above each of these exemplary ranges also can be achieved from the non-naturally occurring cells provided herein. For example, the intracellular concentration of 3-HBal, 1,3-BDO, 4-HBal or 1,4-BDO, or a downstream product related thereto such as an ester or amide thereof, can be between about 100 mM to 1.3 M, including about 100 mM, 200 mM, 500 mM, 800 mM, 1 M, 1.1 M, 1.2 M, 1.3 M, or higher.

A cell provided herein is cultured using well known methods. The culture conditions can include, for example, liquid culture procedures as well as fermentation and other large scale culture procedures. As described herein, particularly useful yields of the biosynthetic products provided herein can be obtained under anaerobic or substantially anaerobic culture conditions.

In some embodiments, culture conditions include anaerobic or substantially anaerobic growth or maintenance conditions. Exemplary anaerobic conditions have been described previously and are well known in the art. Exemplary anaerobic conditions for fermentation processes are described herein and are described, for example, in U.S. publication 2009/0047719, filed Aug. 10, 2007. Any of these conditions can be employed with the non-naturally occurring cells as well as other anaerobic conditions well known in the art. Under such anaerobic or substantially anaerobic conditions, the 3-HBal, 1,3-BDO, 4-HBal or 1,4-BDO producers can synthesize 3-HBal, 1,3-BDO, 4-HBal or 1,4-BDO, or a downstream product related thereto such as an ester or amide thereof, at intracellular concentrations of 5-10 mM or more as well as all other concentrations exemplified herein. It is understood that, even though the above description refers to intracellular concentrations, 3-HBal, 1,3-BDO, 4-HBal or 1,4-BDO producing cells can produce 3-HBal, 1,3-BDO, 4-HBal or 1,4-BDO, or a downstream product related thereto such as an ester or amide thereof, intracellularly and/or secrete the product into the culture medium.

As described herein, one exemplary growth condition for achieving biosynthesis of 3-HBal, 1,3-BDO, 4-HBal or 1,4-BDO, or a downstream product related thereto such as an ester or amide thereof, includes anaerobic culture or fermentation conditions. In certain embodiments, the non-naturally occurring cells provided herein can be sustained, cultured or fermented under anaerobic or substantially anaerobic conditions. Briefly, an anaerobic condition refers to an environment devoid of oxygen. Substantially anaerobic conditions include, for example, a culture, batch fermentation or continuous fermentation such that the dissolved oxygen concentration in the medium remains between 0 and 10% of saturation. Substantially anaerobic conditions also includes growing or resting cells in liquid medium or on solid agar inside a sealed chamber maintained with an atmosphere of less than 1% oxygen. The percent of oxygen can be maintained by, for example, sparging the culture with an N₂/CO₂ mixture or other suitable non-oxygen gas or gases.

The culture conditions described herein can be scaled up and grown continuously for manufacturing of 3-HBal, 1,3-BDO, 4-HBal or 1,4-BDO, or a downstream product related thereto such as an ester or amide thereof, by a cell provided herein. Exemplary growth procedures include, for example, fed-batch fermentation and batch separation; fed-batch fermentation and continuous separation, or continuous fermentation and continuous separation. All of these processes are well known in the art. Fermentation procedures are particularly useful for the biosynthetic production of commercial quantities of 3-HBal, 1,3-BDO, 4-HBal or 1,4-BDO, or a downstream product related thereto such as an ester or amide thereof. Generally, and as with non-continuous culture procedures, the continuous and/or near-continuous production of 3-HBal, 1,3-BDO, 4-HBal or 1,4-BDO, or a downstream product related thereto such as an ester or amide thereof, will include culturing a non-naturally occurring cell producing 3-HBal, 1,3-BDO, 4-HBal or 1,4-BDO, or a downstream product related thereto such as an ester or amide thereof, provided herein in sufficient nutrients and medium to sustain and/or nearly sustain growth in an exponential phase. Continuous culture under such conditions can include, for example, growth or culturing for 1 day, 2, 3, 4, 5, 6 or 7 days or more. Additionally, continuous culture can include longer time periods of 1 week, 2, 3, 4 or 5 or more weeks and up to several months. Alternatively, organisms provided herein can be cultured for hours, if suitable for a particular application. It is to be understood that the continuous and/or near-continuous culture conditions also can include all time intervals in between these exemplary periods. It is further understood that the time of culturing the cell provided herein is for a sufficient period of time to produce a sufficient amount of product for a desired purpose.

Exemplary fermentation processes include, but are not limited to, fed-batch fermentation and batch separation; fed-batch fermentation and continuous separation; and continuous fermentation and continuous separation. In an exemplary batch fermentation protocol, the production organism is grown in a suitably sized bioreactor sparged with an appropriate gas. Under anaerobic conditions, the culture is sparged with an inert gas or combination of gases, for example, nitrogen, N₂/CO₂ mixture, argon, helium, and the like. As the cells grow and utilize the carbon source, additional carbon source(s) and/or other nutrients are fed into the bioreactor at a rate approximately balancing consumption of the carbon source and/or nutrients. The temperature of the bioreactor is maintained at a desired temperature, generally in the range of 22-37 degrees C., but the temperature can be maintained at a higher or lower temperature depending on the growth characteristics of the production organism and/or desired conditions for the fermentation process. Growth continues for a desired period of time to achieve desired characteristics of the culture in the fermenter, for example, cell density, product concentration, and the like. In a batch fermentation process, the time period for the fermentation is generally in the range of several hours to several days, for example, 8 to 24 hours, or 1, 2, 3, 4 or 5 days, or up to a week, depending on the desired culture conditions. The pH can be controlled or not, as desired, in which case a culture in which pH is not controlled will typically decrease to pH 3-6 by the end of the run. Upon completion of the cultivation period, the fermenter contents can be passed through a cell separation unit, for example, a centrifuge, filtration unit, and the like, to remove cells and cell debris. In the case where the desired product is expressed intracellularly, the cells can be lysed or disrupted enzymatically or chemically prior to or after separation of cells from the fermentation broth, as desired, in order to release additional product. The fermentation broth can be transferred to a product separations unit. Isolation of product occurs by standard separations procedures employed in the art to separate a desired product from dilute aqueous solutions. Such methods include, but are not limited to, liquid-liquid extraction using a water immiscible organic solvent (e.g., toluene or other suitable solvents, including but not limited to diethyl ether, ethyl acetate, tetrahydrofuran (THF), methylene chloride, chloroform, benzene, pentane, hexane, heptane, petroleum ether, methyl tertiary butyl ether (MTBE), dioxane, dimethylformamide (DMF), dimethyl sulfoxide (DMSO), and the like) to provide an organic solution of the product, if appropriate, standard distillation methods, and the like, depending on the chemical characteristics of the product of the fermentation process.

In an exemplary fully continuous fermentation protocol, the production organism is generally first grown up in batch mode in order to achieve a desired cell density. When the carbon source and/or other nutrients are exhausted, feed medium of the same composition is supplied continuously at a desired rate, and fermentation liquid is withdrawn at the same rate. Under such conditions, the product concentration in the bioreactor generally remains constant, as well as the cell density. The temperature of the fermenter is maintained at a desired temperature, as discussed above. During the continuous fermentation phase, it is generally desirable to maintain a suitable pH range for optimized production. The pH can be monitored and maintained using routine methods, including the addition of suitable acids or bases to maintain a desired pH range. The bioreactor is operated continuously for extended periods of time, generally at least one week to several weeks and up to one month, or longer, as appropriate and desired. The fermentation liquid and/or culture is monitored periodically, including sampling up to every day, as desired, to assure consistency of product concentration and/or cell density. In continuous mode, fermenter contents are constantly removed as new feed medium is supplied. The exit stream, containing cells, medium, and product, are generally subjected to a continuous product separations procedure, with or without removing cells and cell debris, as desired. Continuous separations methods employed in the art can be used to separate the product from dilute aqueous solutions, including but not limited to continuous liquid-liquid extraction using a water immiscible organic solvent (e.g., toluene or other suitable solvents, including but not limited to diethyl ether, ethyl acetate, tetrahydrofuran (THF), methylene chloride, chloroform, benzene, pentane, hexane, heptane, petroleum ether, methyl tertiary butyl ether (MTBE), dioxane, dimethylformamide (DMF), dimethyl sulfoxide (DMSO), and the like), standard continuous distillation methods, and the like, or other methods well known in the art.

Fermentation procedures are well known in the art. Briefly, fermentation for the biosynthetic production of 3-HBal, 1,3-BDO, 4-HBal or 1,4-BDO, or a downstream product related thereto such as an ester or amide thereof, can be utilized in, for example, fed-batch fermentation and batch separation; fed-batch fermentation and continuous separation, or continuous fermentation and continuous separation. Examples of batch and continuous fermentation procedures are well known in the art and described herein.

In addition to the fermentation procedures described herein using the producers of 3-HBal, 1,3-BDO, 4-HBal or 1,4-BDO, or a downstream product related thereto such as an ester or amide thereof, provided herein for continuous production of substantial quantities of 3-HBal, 1,3-BDO, 4-HBal or 1,4-BDO, or a downstream product related thereto such as an ester or amide thereof, the 3-HBal, 1,3-BDO, 4-HBal or 1,4-BDO, or a downstream product related thereto such as an ester or amide, producers also can be, for example, simultaneously subjected to chemical synthesis and/or enzymatic procedures to convert the product to other compounds, or the product can be separated from the fermentation culture and sequentially subjected to chemical and/or enzymatic conversion to convert the product to other compounds, if desired.

In some embodiments, the carbon feedstock and other cellular uptake sources such as phosphate, ammonia, sulfate, chloride and other halogens can be chosen to alter the isotopic distribution of the atoms present in 3-HBal, 1,3-BDO, 4-HBal or 1,4-BDO, or a downstream product related thereto such as an ester or amide thereof, or any 3-HBal, 1,3-BDO, 4-HBal or 1,4-BDO pathway intermediate. The various carbon feedstock and other uptake sources enumerated above will be referred to herein, collectively, as “uptake sources.” Uptake sources can provide isotopic enrichment for any atom present in the product 3-HBal, 1,3-BDO, 4-HBal or 1,4-BDO, or a downstream product related thereto such as an ester or amide thereof, or 3-HBal, 1,3-BDO, 4-HBal or 1,4-BDO pathway intermediate, or for side products generated in reactions diverging away from a 3-HBal, 1,3-BDO, 4-HBal or 1,4-BDO pathway. Isotopic enrichment can be achieved for any target atom including, for example, carbon, hydrogen, oxygen, nitrogen, sulfur, phosphorus, chloride or other halogens.

In some embodiments, the uptake sources can be selected to alter the carbon-12, carbon-13, and carbon-14 ratios. In some embodiments, the uptake sources can be selected to alter the oxygen-16, oxygen-17, and oxygen-18 ratios. In some embodiments, the uptake sources can be selected to alter the hydrogen, deuterium, and tritium ratios. In some embodiments, the uptake sources can be selected to alter the nitrogen-14 and nitrogen-15 ratios. In some embodiments, the uptake sources can be selected to alter the sulfur-32, sulfur-33, sulfur-34, and sulfur-35 ratios. In some embodiments, the uptake sources can be selected to alter the phosphorus-31, phosphorus-32, and phosphorus-33 ratios. In some embodiments, the uptake sources can be selected to alter the chlorine-35, chlorine-36, and chlorine-37 ratios.

In some embodiments, the isotopic ratio of a target atom can be varied to a desired ratio by selecting one or more uptake sources. An uptake source can be derived from a natural source, as found in nature, or from a man-made source, and one skilled in the art can select a natural source, a man-made source, or a combination thereof, to achieve a desired isotopic ratio of a target atom. An example of a man-made uptake source includes, for example, an uptake source that is at least partially derived from a chemical synthetic reaction. Such isotopically enriched uptake sources can be purchased commercially or prepared in the laboratory and/or optionally mixed with a natural source of the uptake source to achieve a desired isotopic ratio. In some embodiments, a target atom isotopic ratio of an uptake source can be achieved by selecting a desired origin of the uptake source as found in nature. For example, as discussed herein, a natural source can be a biobased source derived from or synthesized by a biological organism or a source such as petroleum-based products or the atmosphere. In some such embodiments, a source of carbon, for example, can be selected from a fossil fuel-derived carbon source, which can be relatively depleted of carbon-14, or an environmental or atmospheric carbon source, such as CO₂, which can possess a larger amount of carbon-14 than its petroleum-derived counterpart.

The unstable carbon isotope carbon-14 or radiocarbon makes up for roughly 1 in 10¹² carbon atoms in the earth's atmosphere and has a half-life of about 5700 years. The stock of carbon is replenished in the upper atmosphere by a nuclear reaction involving cosmic rays and ordinary nitrogen (¹⁴N). Fossil fuels contain no carbon-14, as it decayed long ago. Burning of fossil fuels lowers the atmospheric carbon-14 fraction, the so-called “Suess effect”.

Methods of determining the isotopic ratios of atoms in a compound are well known to those skilled in the art. Isotopic enrichment is readily assessed by mass spectrometry using techniques known in the art such as accelerated mass spectrometry (AMS), Stable Isotope Ratio Mass Spectrometry (SIRMS) and Site-Specific Natural Isotopic Fractionation by Nuclear Magnetic Resonance (SNIF-NMR). Such mass spectral techniques can be integrated with separation techniques such as liquid chromatography (LC), high performance liquid chromatography (HPLC) and/or gas chromatography, and the like.

In the case of carbon, ASTM D6866 was developed in the United States as a standardized analytical method for determining the biobased content of solid, liquid, and gaseous samples using radiocarbon dating by the American Society for Testing and Materials (ASTM) International. The standard is based on the use of radiocarbon dating for the determination of a product's biobased content. ASTM D6866 was first published in 2004, and the current active version of the standard is ASTM D6866-11 (effective Apr. 1, 2011). Radiocarbon dating techniques are well known to those skilled in the art, including those described herein.

The biobased content of a compound is estimated by the ratio of carbon-14 (¹⁴C) to carbon-12 (¹²C). Specifically, the Fraction Modern (Fm) is computed from the expression: Fm=(S−B)/(M−B), where B, S and M represent the ¹⁴C/¹²C ratios of the blank, the sample and the modern reference, respectively. Fraction Modern is a measurement of the deviation of the ¹⁴C/¹²C ratio of a sample from “Modern.” Modern is defined as 95% of the radiocarbon concentration (in AD 1950) of National Bureau of Standards (NBS) Oxalic Acid I (i.e., standard reference materials (SRM) 4990b) normalized to δ¹³C_{VPDB}=−19 per mil (Olsson, The use of Oxalic acid as a Standard. in, Radiocarbon Variations and Absolute Chronology, Nobel Symposium, 12th Proc., John Wiley & Sons, New York (1970)). Mass spectrometry results, for example, measured by ASM, are calculated using the internationally agreed upon definition of 0.95 times the specific activity of NBS Oxalic Acid I (SRM 4990b) normalized to δ¹³C_{VPDB}=−19 per mil. This is equivalent to an absolute (AD 1950)¹⁴C/¹²C ratio of 1.176 0.010×10⁻¹² (Karlen et al., Arkiv Geofysik, 4:465-471 (1968)). The standard calculations take into account the differential uptake of one isotope with respect to another, for example, the preferential uptake in biological systems of C¹² over C¹³ over C¹⁴, and these corrections are reflected as a Fm corrected for δ¹³

An oxalic acid standard (SRM 4990b or HOx 1) was made from a crop of 1955 sugar beet. Although there were 1000 lbs made, this oxalic acid standard is no longer commercially available. The Oxalic Acid II standard (HOx 2; N.I.S.T designation SRM 4990 C) was made from a crop of 1977 French beet molasses. In the early 1980's, a group of 12 laboratories measured the ratios of the two standards. The ratio of the activity of Oxalic acid II to 1 is 1.2933±0.001 (the weighted mean). The isotopic ratio of HOx II is −17.8 per mil. ASTM D6866-11 suggests use of the available Oxalic Acid II standard SRM 4990 C (Hox2) for the modern standard (see discussion of original vs. currently available oxalic acid standards in Mann, Radiocarbon, 25(2):519-527 (1983)). A Fm=0% represents the entire lack of carbon-14 atoms in a material, thus indicating a fossil (for example, petroleum based) carbon source. A Fm=100%, after correction for the post-1950 injection of carbon-14 into the atmosphere from nuclear bomb testing, indicates an entirely modern carbon source. As described herein, such a “modern” source includes biobased sources.

As described in ASTM D6866, the percent modern carbon (pMC) can be greater than 100% because of the continuing but diminishing effects of the 1950s nuclear testing programs, which resulted in a considerable enrichment of carbon-14 in the atmosphere as described in ASTM D6866-11. Because all sample carbon-14 activities are referenced to a “pre-bomb” standard, and because nearly all new biobased products are produced in a post-bomb environment, all pMC values (after correction for isotopic fraction) must be multiplied by 0.95 (as of 2010) to better reflect the true biobased content of the sample. A biobased content that is greater than 103% suggests that either an analytical error has occurred, or that the source of biobased carbon is more than several years old.

ASTM D6866 quantifies the biobased content relative to the material's total organic content and does not consider the inorganic carbon and other non-carbon containing substances present. For example, a product that is 50% starch-based material and 50% water would be considered to have a Biobased Content=100% (50% organic content that is 100% biobased) based on ASTM D6866. In another example, a product that is 50% starch-based material, 25% petroleum-based, and 25% water would have a Biobased Content=66.7% (75% organic content but only 50% of the product is biobased). In another example, a product that is 50% organic carbon and is a petroleum-based product would be considered to have a Biobased Content=0% (50% organic carbon but from fossil sources). Thus, based on the well known methods and known standards for determining the biobased content of a compound or material, one skilled in the art can readily determine the biobased content of a compound or material and/or prepared downstream products that utilize a compound or material provided herein having a desired biobased content.

Applications of carbon-14 dating techniques to quantify bio-based content of materials are known in the art (Currie et al., Nuclear Instruments and Methods in Physics Research B, 172:281-287 (2000)). For example, carbon-14 dating has been used to quantify bio-based content in terephthalate-containing materials (Colonna et al., Green Chemistry, 13:2543-2548 (2011)). Notably, polypropylene terephthalate (PPT) polymers derived from renewable 1,3-propanediol and petroleum-derived terephthalic acid resulted in Fm values near 30% (i.e., since 3/11 of the polymeric carbon derives from renewable 1,3-propanediol and 8/11 from the fossil end member terephthalic acid) (Currie et al., supra, 2000). In contrast, polybutylene terephthalate polymer derived from both renewable 1,4-butanediol and renewable terephthalic acid resulted in bio-based content exceeding 90% (Colonna et al., supra, 2011).

Accordingly, in some embodiments, provided herein is 3-HBal, 1,3-BDO, 4-HBal or 1,4-BDO or a downstream product related thereto such as an ester or amide thereof, or a 3-HBal, 1,3-BDO, 4-HBal or 1,4-BDO pathway intermediate, produced by a cell provided herein, that has a carbon-12, carbon-13, and carbon-14 ratio that reflects an atmospheric carbon, also referred to as environmental carbon, uptake source. For example, in some aspects the 3-HBal, 1,3-BDO, 4-HBal or 1,4-BDO, or a downstream product related thereto such as an ester or amide thereof, or a 3-HBal, 1,3-BDO, 4-HBal or 1,4-BDO pathway intermediate can have an Fm value of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or as much as 100%. In some such embodiments, the uptake source is CO₂. In some embodiments, provided herein is 3-HBal, 1,3-BDO, 4-HBal or 1,4-BDO, or a downstream product related thereto such as an ester or amide thereof, or a 3-HBal, 1,3-BDO, 4-HBal or 1,4-BDO pathway intermediate that has a carbon-12, carbon-13, and carbon-14 ratio that reflects petroleum-based carbon uptake source. In this aspect, the 3-HBal, 1,3-BDO, 4-HBal or 1,4-BDO, or a downstream product related thereto such as an ester or amide thereof, or a 3-HBal, 1,3-BDO, 4-HBal or 1,4-BDO pathway intermediate can have an Fm value of less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 2% or less than 1%. In some embodiments, provided herein is 3-HBal, 1,3-BDO, 4-HBal or 1,4-BDO, or a downstream product related thereto such as an ester or amide thereof, or a 3-HBal, 1,3-BDO, 4-HBal or 1,4-BDO pathway intermediate that has a carbon-12, carbon-13, and carbon-14 ratio that is obtained by a combination of an atmospheric carbon uptake source with a petroleum-based uptake source. Using such a combination of uptake sources is one way by which the carbon-12, carbon-13, and carbon-14 ratio can be varied, and the respective ratios would reflect the proportions of the uptake sources.

Further, the compositions provided herein relate to the biologically produced 3-HBal, 1,3-BDO, 4-HBal or 1,4-BDO, or a downstream product related thereto such as an ester or amide thereof, or 3-HBal, 1,3-BDO, 4-HBal or 1,4-BDO pathway intermediate as disclosed herein, and to the products derived therefrom, wherein the 3-HBal, 1,3-BDO, 4-HBal or 1,4-BDO, or a downstream product related thereto such as an ester or amide thereof, or a 3-HBal, 1,3-BDO, 4-HBal or 1,4-BDO pathway intermediate has a carbon-12, carbon-13, and carbon-14 isotope ratio of about the same value as the CO₂ that occurs in the environment. For example, in some aspects provided herein is bioderived 3-HBal, 1,3-BDO, 4-HBal or 1,4-BDO, or a downstream product related thereto such as an ester or amide thereof, or a bioderived 3-HBal, 1,3-BDO, 4-HBal of 1,4-BDO intermediate having a carbon-12 versus carbon-13 versus carbon-14 isotope ratio of about the same value as the CO₂ that occurs in the environment, or any of the other ratios disclosed herein. It is understood, as disclosed herein, that a product can have a carbon-12 versus carbon-13 versus carbon-14 isotope ratio of about the same value as the CO₂ that occurs in the environment, or any of the ratios disclosed herein, wherein the product is generated from bioderived 3-HBal, 1,3-BDO, 4-HBal or 1,4-BDO, or a downstream product related thereto such as an ester or amide thereof, or a bioderived 3-HBal, 1,3-BDO, 4-HBal or 1,4-BDO pathway intermediate as disclosed herein, wherein the bioderived product is chemically modified to generate a final product. Methods of chemically modifying a bioderived product of 3-HBal, 1,3-BDO, 4-HBal or 1,4-BDO, or a downstream product related thereto such as an ester or amide thereof, or an intermediate of a 3-HBal, 1,3-BDO, 4-HBal or 1,4-BDO, to generate a desired product are well known to those skilled in the art, as described herein. Further provided herein is plastics, elastic fibers, polyurethanes, polyesters, including polyhydroxyalkanoates, nylons, organic solvents, polyurethane resins, polyester resins, hypoglycaemic agents, butadiene and/or butadiene-based products, which can be based on 3-HBal and/or 1,3-BDO, or a downstream product related thereto such as an ester or amide thereof, and plastics, elastic fibers, polyurethanes, polyesters, including polyhydroxyalkanoates such as poly-4-hydroxybutyrate (P4HB) or co-polymers thereof, poly(tetramethylene ether) glycol (PTMEG)(also referred to as PTMO, polytetramethylene oxide), polybutylene terephthalate (PBT), and polyurethane-polyurea copolymers, referred to as spandex, elastane or Lycra™, nylons, and the like, which can be based on 4-HBal and/or 1,4-BDO, or a downstream product related thereto such as an ester or amide thereof, having a carbon-12 versus carbon-13 versus carbon-14 isotope ratio of about the same value as the CO₂ that occurs in the environment, wherein the plastics, elastic fibers, polyurethanes, polyesters, including polyhydroxyalkanoates such as poly-4-hydroxybutyrate (P4HB) or co-polymers thereof, poly(tetramethylene ether) glycol (PTMEG)(also referred to as PTMO, polytetramethylene oxide), polybutylene terephthalate (PBT), and polyurethane-polyurea copolymers, referred to as spandex, elastane or Lycra™, nylons, organic solvents, polyurethane resins, polyester resins, hypoglycaemic agents, butadiene, and/or butadiene-based products are generated directly from or in combination with bioderived 3-HBal, 1,3-BDO, 4-HBal or 1,4-BDO, or a downstream product related thereto such as an ester or amide thereof, or a bioderived 3-HBal, 1,3-BDO, 4-HBal or 1,4-BDO pathway intermediate as disclosed herein. Methods for producing butadiene and/or butadiene-based products have been described previously (see, for example, WO 2010/127319, WO 2013/036764, U.S. Pat. No. 9,017,983, US 2013/0066035, WO/2012/018624, US 2012/0021478, each of which is incorporated herein by reference). 1,3-BDO can be reacted with an acid, either in vivo or in vitro, to convert to an ester using, for example, a lipase. Such esters can have nutraceutical, pharmaceutical and food uses, and are advantaged when R-form of 1,3-BDO is used since that is the form (compared to S-form or the racemic mixture) best utilized by both animals and humans as an energy source (e.g., a ketone ester, such as (R)-3-hydroxybutyl-R-1,3-butanediol monoester (which has Generally Recognized As Safe (GRAS) approval in the United States) and (R)-3-hydroxybutyrate glycerol monoester or diester). The ketone esters can be delivered orally, and the ester releases R-1,3-butanediol that is used by the body (see, for example, WO2013150153). Methods of producing amides are well known in the art (see, for example, Goswami and Van Lanen, Mol. Biosyst. 11(2):338-353 (2015)).

Thus, the engineered aldehyde dehydrogenase provided herein is particularly useful to provide an improved enzymatic route and microorganism to provide an improved composition of 1,3-BDO, namely R-1,3-butanediol, highly enriched or essentially enantiomerically pure, and further having improved purity qualities with respect to by-products. 1,3-BDO has further food related uses including use directly as a food source, a food ingredient, a flavoring agent, a solvent or solubilizer for flavoring agents, a stabilizer, an emulsifier, and an anti-microbial agent and preservative. 1,3-BDO is used in the pharmaceutical industry as a parenteral drug solvent. 1,3-BDO finds use in cosmetics as an ingredient that is an emollient, a humectant, that prevents crystallization of insoluble ingredients, a solubilizer for less-water-soluble ingredients such as fragrances, and as an anti-microbial agent and preservative. For example, it can be used as a humectant, especially in hair sprays and setting lotions; it reduces loss of aromas from essential oils, preserves against spoilage by microorganisms, and is used as a solvent for benzoates. 1,3-BDO can be used at concentrations from 0.1% to 50%, and even less than 0.1% and even more than 50%. It is used in hair and bath products, eye and facial makeup, fragrances, personal cleanliness products, and shaving and skin care preparations (see, for example, the Cosmetic Ingredient Review board's report: “Final Report on the Safety Assessment of Butylene Glycol, Hexylene Glycol, Ethoxydiglycol, and Dipropylene Glycol”, Journal of the American College of Toxicology, Volume 4, Number 5, 1985, which is incorporated herein by reference). This report provides specific uses and concentrations of 1,3-BDO in cosmetics; see for examples the report's Table 2 therein entitled “Product Formulation Data”.

In one embodiment, provided herein is a culture medium comprising bioderived 3-HBal and/or 1,3-BDO, or 4-HBal and/or 1,4-BDO, wherein the bioderived 3-HBal and/or 1,3-BDO, or 4-HBal and/or 1,4-BDO, has a carbon-12, carbon-13 and carbon-14 isotope ratio that reflects an atmospheric carbon dioxide uptake source, and wherein the bioderived 3-HBal and/or 1,3-BDO, or 4-HBal and/or 1,4-BDO is produced by a cell, or in a cell lysate, provided herein or a method provided herein. In one embodiment, the culture medium is separated from the cell.

In one embodiment, provided herein is 3-hydroxybutyraldeyde (3-HBal) and/or 1,3-butanediol (1,3-BDO), or 4-hydroxybutyraldeyde (4-HBal) and/or 1,4-butanediol (1,4-BDO), having a carbon-12, carbon-13 and carbon-14 isotope ratio that reflects an atmospheric carbon dioxide uptake source, wherein the 3-HBal and/or 1,3-BDO, or the 4-HBal and/or 1,4-BDO, is produced by a cell, or in a cell lysate, provided herein or a method provided herein. In one embodiment, the 3-HBal and/or 1,3-BDO, or the 4-HBal and/or 1,4-BDO, has an Fm value of at least 80%, at least 85%, at least 90%, at least 95% or at least 98%.

In one embodiment, provided herein is 3-hydroxybutyraldehyde (3-HBal) and/or 1,3-butanediol (1,3-BDO), or 4-hydroxybutyraldehyde (4-HBal) and/or 1,4-butanediol (1,4-BDO), produced by a cell, or in a cell lysate provided herein or a method provided herein. In one embodiment, provided herein is 3-hydroxybutyraldeyde (3-HBal) and/or 1,3-butanediol (1,3-BDO) having a carbon-12, carbon-13 and carbon-14 isotope ratio that reflects an atmospheric carbon dioxide uptake source, wherein the 3-HBal and/or 1,3-BDO is produced by a cell, or in a cell lysate, provided herein or a method provided herein, wherein the 3-HBal and/or 1,3-BDO is enantiomerically enriched for the R form. In one embodiment, the 3-HBal and/or 1,3-BDO has an Fm value of at least 80%, at least 85%, at least 90%, at least 95% or at least 98%.

In one embodiment, provided herein is 3-hydroxybutyraldehyde (3-HBal) and/or 1,3-butanediol (1,3-BDO) produced by a cell, or in a cell lysate, provided herein or a method provided herein, wherein the 3-HBal and/or 1,3-BDO is enantiomerically enriched for the R form. In one embodiment, the R form is greater than 95%, 96%, 97%, 98%, 99%, 99.1%, 99.2%, 99.3%, 99.4%, 99.5%, 99.6%, 99.7%, 99.8% or 99.9% of the 3-HBal and/or 1,3-BDO. In one embodiment, the 3-HBal and/or 1,3-BDO is ≥55% R-enantiomer, ≥60% R-enantiomer, ≥65% R-enantiomer, ≥70% R-enantiomer, ≥75% R-enantiomer, ≥80% R-enantiomer, ≥85% R-enantiomer, ≥90% R-enantiomer, or ≥95% R-enantiomer, and can be highly chemically pure, e.g., ≥99%, for example, ≥95%, ≥96%, ≥97%, ≥98%, ≥99%, ≥99.1%, ≥99.2%, ≥99.3%, ≥99.4%, ≥99.5%, ≥99.6%, ≥99.7%, ≥99.8% or ≥99.9% R-enantiomer.

In one embodiment, provided herein is a composition comprising 3-HBal and/or 1,3-BDO, or the 4-HBal and/or 1,4-BDO, produced by a cell, or in a cell lysate, provided herein or a method provided herein and a compound other than the 3-HBal and/or 1,3-BDO, or 4-HBal or 1,4-BDO, respectively. In one embodiment, the compound other than the 3-HBal and/or 1,3-BDO, or the 4-HBal and/or 1,4-BDO, is a portion of a cell that produces the 3-HBal and/or 1,3-BDO, or the 4-HBal and/or 1,4-BDO, respectively, or that expresses an engineered aldehyde dehydrogenase provided herein.

In one embodiment, provided herein is a composition comprising 3-HBal and/or 1,3-BDO, or the 4-HBal and/or 1,4-BDO, produced by a cell, or in a cell lysate, provided herein or a method provided herein, or a cell lysate or culture supernatant of a cell producing the 3-HBal and/or 1,3-BDO, or the 4-HBal and/or 1,4-BDO.

In one embodiment, provided herein is a product comprising 3-HBal and/or 1,3-BDO, or the 4-HBal and/or 1,4-BDO, produced by a cell, or in a cell lysate provided herein or a method provided herein, wherein the product is a plastic, elastic fiber, polyurethane, polyester, polyhydroxyalkanoate, poly-4-hydroxybutyrate (P4HB) or a co-polymer thereof, poly(tetramethylene ether) glycol (PTMEG), polybutylene terephthalate (PBT), polyurethane-polyurea copolymer, nylon, organic solvent, polyurethane resin, polyester resin, hypoglycaemic agent, butadiene or butadiene-based product. In one embodiment, the product is a cosmetic product or a food additive. In one embodiment, the product comprises at least 0.1%, at least 0.5%, at least 1%, at least 5%, at least 10%, at least 20%, at least 30%, at least 40% or at least 50% bioderived 3-HBal and/or 1,3-BDO, or bioderived 4-HBal and/or 1,4-BDO. In one embodiment, the product comprises a portion of the produced 3-HBal and/or 1,3-BDO, or the produced 4-HBal and/or 1,4-BDO, as a repeating unit. In one embodiment, provided herein is a molded product obtained by molding a product made with or derived from 3-HBal and/or 1,3-BDO, or 4-HBal and/or 1,4-BDO produced by a cell, or in a cell lysate provided herein or a method provided herein.

Further provided herein is a composition comprising bioderived 3-HBal, 1,3-BDO, 4-HBal or 1,4-BDO, or a downstream product related thereto such as an ester or amide thereof, and a compound other than the bioderived 3-HBal, 1,3-BDO, 4-HBal or 1,4-BDO, or a downstream product related thereto such as an ester or amide thereof. The compound other than the bioderived product can be a cellular portion, for example, a trace amount of a cellular portion of, or can be fermentation broth or culture medium or a purified or partially purified fraction thereof produced in the presence of, a non-naturally occurring cell provided herein having a pathway that produces 3-HBal, 1,3-BDO, 4-HBal or 1,4-BDO, or a downstream product related thereto such as an ester or amide thereof. The composition can comprise, for example, a reduced level of a byproduct when produced by an organism having reduced byproduct formation, as disclosed herein. The composition can comprise, for example, bioderived 3-HBal, 1,3-BDO, 4-HBal or 1,4-BDO, or a downstream product related thereto such as an ester or amide thereof, or a cell lysate or culture supernatant of a cell provided herein.

3-HBal, 1,3-BDO, 4-HBal or 1,4-BDO, or a downstream product related thereto such as an ester or amide thereof, is a chemical used in commercial and industrial applications. Non-limiting examples of such applications include production of plastics, elastic fibers, polyurethanes, polyesters, including polyhydroxyalkanoates such as poly-4-hydroxybutyrate (P4HB) or co-polymers thereof, poly(tetramethylene ether) glycol (PTMEG)(also referred to as PTMO, polytetramethylene oxide), polybutylene terephthalate (PBT), and polyurethane-polyurea copolymers, referred to as spandex, elastane or Lycra™, nylons, organic solvents, polyurethane resins, polyester resins, hypoglycaemic agents, butadiene and/or butadiene-based products. Moreover, 3-HBal, 1,3-BDO, 4-HBal or 1,4-BDO is also used as a raw material in the production of a wide range of products including plastics, elastic fibers, polyurethanes, polyesters, including polyhydroxyalkanoates such as poly-4-hydroxybutyrate (P4HB) or co-polymers thereof, poly(tetramethylene ether) glycol (PTMEG)(also referred to as PTMO, polytetramethylene oxide), polybutylene terephthalate (PBT), and polyurethane-polyurea copolymers, referred to as spandex, elastane or Lycra™, nylons, organic solvents, polyurethane resins, polyester resins, hypoglycaemic agents, butadiene and/or butadiene-based products. Accordingly, in some embodiments, provided herein is biobased plastics, elastic fibers, polyurethanes, polyesters, including polyhydroxyalkanoates such as poly-4-hydroxybutyrate (P4HB) or co-polymers thereof, poly(tetramethylene ether) glycol (PTMEG)(also referred to as PTMO, polytetramethylene oxide), polybutylene terephthalate (PBT), and polyurethane-polyurea copolymers, referred to as spandex, elastane or Lycra™, nylons, organic solvents, polyurethane resins, polyester resins, hypoglycaemic agents, butadiene and/or butadiene-based products comprising one or more bioderived 3-HBal, 1,3-BDO, 4-HBal or 1,4-BDO, or a downstream product related thereto such as an ester or amide thereof, or bioderived 3-HBal, 1,3-BDO, 4-HBal or 1,4-BDO pathway intermediate produced by a non-naturally occurring cell provided herein, for example, expressing an engineered aldehyde dehydrogenase provided herein, or produced using a method disclosed herein.

In some embodiments, provided herein is plastics, elastic fibers, polyurethanes, polyesters, including polyhydroxyalkanoates such as poly-4-hydroxybutyrate (P4HB) or co-polymers thereof, poly(tetramethylene ether) glycol (PTMEG)(also referred to as PTMO, polytetramethylene oxide), polybutylene terephthalate (PBT), and polyurethane-polyurea copolymers, referred to as spandex, elastane or Lycra™, nylons, organic solvents, polyurethane resins, polyester resins, hypoglycaemic agents, butadiene and/or butadiene-based products comprising bioderived 3-HBal, 1,3-BDO, 4-HBal or 1,4-BDO, or a downstream product related thereto such as an ester or amide thereof, or bioderived 3-HBal, 1,3-BDO, 4-HBal or 1,4-BDO pathway intermediate, wherein the bioderived 3-HBal, 1,3-BDO, 4-HBal or 1,4-BDO, or a downstream product related thereto such as an ester or amide thereof, or bioderived 3-HBal, 1,3-BDO, 4-HBal or 1,4-BDO pathway intermediate includes all or part of the 3-HBal, 1,3-BDO, 4-HBal or 1,4-BDO, or a downstream product related thereto such as an ester or amide thereof, or 3-HBal, 1,3-BDO, 4-HBal or 1,4-BDO pathway intermediate used in the production of plastics, elastic fibers, polyurethanes, polyesters, including polyhydroxyalkanoates such as poly-4-hydroxybutyrate (P4HB) or co-polymers thereof, poly(tetramethylene ether) glycol (PTMEG)(also referred to as PTMO, polytetramethylene oxide), polybutylene terephthalate (PBT), and polyurethane-polyurea copolymers, referred to as spandex, elastane or Lycra™, nylons, organic solvents, polyurethane resins, polyester resins, hypoglycaemic agents, butadiene and/or butadiene-based products. For example, the final plastics, elastic fibers, polyurethanes, polyesters, including polyhydroxyalkanoates such as poly-4-hydroxybutyrate (P4HB) or co-polymers thereof, poly(tetramethylene ether) glycol (PTMEG)(also referred to as PTMO, polytetramethylene oxide), polybutylene terephthalate (PBT), and polyurethane-polyurea copolymers, referred to as spandex, elastane or Lycra™, nylons, organic solvents, polyurethane resins, polyester resins, hypoglycaemic agents, butadiene and/or butadiene-based products can contain the bioderived 3-HBal, 1,3-BDO, 4-HBal or 1,4-BDO, or a downstream product related thereto such as an ester or amide thereof, or 3-HBal, 1,3-BDO, 4-HBal or 1,4-BDO pathway intermediate, or a portion thereof that is the result of the manufacturing of plastics, elastic fibers, polyurethanes, polyesters, including polyhydroxyalkanoates such as poly-4-hydroxybutyrate (P4HB) or co-polymers thereof, poly(tetramethylene ether) glycol (PTMEG)(also referred to as PTMO, polytetramethylene oxide), polybutylene terephthalate (PBT), and polyurethane-polyurea copolymers, referred to as spandex, elastane or Lycra™, nylons, organic solvents, polyurethane resins, polyester resins, hypoglycaemic agents, butadiene and/or butadiene-based products. Such manufacturing can include chemically reacting the bioderived 3-HBal, 1,3-BDO, 4-HBal or 1,4-BDO, or a downstream product related thereto such as an ester or amide thereof, or bioderived 3-HBal, 1,3-BDO, 4-HBal or 1,4-BDO pathway intermediate (e.g. chemical conversion, chemical functionalization, chemical coupling, oxidation, reduction, polymerization, copolymerization and the like) into the final plastics, elastic fibers, polyurethanes, polyesters, including polyhydroxyalkanoates such as poly-4-hydroxybutyrate (P4HB) or co-polymers thereof, poly(tetramethylene ether) glycol (PTMEG)(also referred to as PTMO, polytetramethylene oxide), polybutylene terephthalate (PBT), and polyurethane-polyurea copolymers, referred to as spandex, elastane or Lycra™, nylons, organic solvents, polyurethane resins, polyester resins, hypoglycaemic agents, butadiene and/or butadiene-based products. Thus, in some aspects, provided herein is a biobased plastic, elastic fiber, polyurethane, polyester, including polyhydroxyalkanoate such as poly-4-hydroxybutyrate (P4HB) or co-polymers thereof, poly(tetramethylene ether) glycol (PTMEG)(also referred to as PTMO, polytetramethylene oxide), polybutylene terephthalate (PBT), and polyurethane-polyurea copolymer, referred to as spandex, elastane or Lycra™, nylon, polyurethane resin, polyester resin, hypoglycaemic agent, butadiene and/or butadiene-based product comprising at least 2%, at least 3%, at least 5%, at least 10%, at least 15%, at least 20%, at least 25%, at least 30%, at least 35%, at least 40%, at least 50%, at least 60%, at least 70%, at least 80%, at least 90%, at least 95%, at least 98% or 100% bioderived 3-HBal, 1,3-BDO, 4-HBal or 1,4-BDO, or a downstream product related thereto such as an ester or amide thereof, or bioderived 3-HBal, 1,3-BDO, 4-HBal or 1,4-BDO pathway intermediate as disclosed herein.

Additionally, in some embodiments, provided herein is a composition having a bioderived 3-HBal, 1,3-BDO, 4-HBal or 1,4-BDO, or a downstream product related thereto such as an ester or amide thereof, or 3-HBal, 1,3-BDO, 4-HBal or 1,4-BDO pathway intermediate disclosed herein and a compound other than the bioderived 3-HBal, 1,3-BDO, 4-HBal or 1,4-BDO, or a downstream product related thereto such as an ester or amide thereof, or 3-HBal, 1,3-BDO, 4-HBal or 1,4-BDO pathway intermediate. For example, in some aspects, provided herein is biobased plastics, elastic fibers, polyurethanes, polyesters, including polyhydroxyalkanoates such as poly-4-hydroxybutyrate (P4HB) or co-polymers thereof, poly(tetramethylene ether) glycol (PTMEG)(also referred to as PTMO, polytetramethylene oxide), polybutylene terephthalate (PBT), and polyurethane-polyurea copolymers, referred to as spandex, elastane or Lycra™, nylons, organic solvents, polyurethane resins, polyester resins, hypoglycaemic agents, butadiene and/or butadiene-based products wherein the 3-HBal, 1,3-BDO, 4-HBal or 1,4-BDO, or a downstream product related thereto such as an ester or amide thereof, or 3-HBal, 1,3-BDO, 4-HBal or 1,4-BDO pathway intermediate used in its production is a combination of bioderived and petroleum derived 3-HBal, 1,3-BDO, 4-HBal or 1,4-BDO, or a downstream product related thereto such as an ester or amide thereof, or 3-HBal, 1,3-BDO, 4-HBal or 1,4-BDO pathway intermediate. For example, biobased plastics, elastic fibers, polyurethanes, polyesters, including polyhydroxyalkanoates such as poly-4-hydroxybutyrate (P4HB) or co-polymers thereof, poly(tetramethylene ether) glycol (PTMEG)(also referred to as PTMO, polytetramethylene oxide), polybutylene terephthalate (PBT), and polyurethane-polyurea copolymers, referred to as spandex, elastane or Lycra™, nylons, organic solvents, polyurethane resins, polyester resins, hypoglycaemic agents, butadiene and/or butadiene-based products can be produced using 50% bioderived 3-HBal, 1,3-BDO, 4-HBal or 1,4-BDO, or a downstream product related thereto such as an ester or amide thereof, and 50% petroleum derived 3-HBal, 1,3-BDO, 4-HBal or 1,4-BDO, or a downstream product related thereto such as an ester or amide thereof, or other desired ratios such as 60%/40%, 70%/30%, 80%/20%, 90%/10%, 95%/5%, 100%/0%, 40%/60%, 30%/70%, 20%/80%, 10%/90% of bioderived/petroleum derived precursors, so long as at least a portion of the product comprises a bioderived product produced by the cells disclosed herein. It is understood that methods for producing plastics, elastic fibers, polyurethanes, polyesters, including polyhydroxyalkanoates such as poly-4-hydroxybutyrate (P4HB) or co-polymers thereof, poly(tetramethylene ether) glycol (PTMEG)(also referred to as PTMO, polytetramethylene oxide), polybutylene terephthalate (PBT), and polyurethane-polyurea copolymers, referred to as spandex, elastane or Lycra™, nylons, organic solvents, polyurethane resins, polyester resins, hypoglycaemic agents, butadiene and/or butadiene-based products using the bioderived 3-HBal, 1,3-BDO, 4-HBal or 1,4-BDO, or a downstream product related thereto such as an ester or amide thereof, or bioderived 3-HBal, 1,3-BDO, 4-HBal or 1,4-BDO pathway intermediate provided herein are well known in the art.

### SEQUENCES

The sequences in the following TABLE 1 illustrate amino acid sequences that can be used to generate the aldehyde dehydrogenase sequences and/or compositions and perform the methods described herein. As needed, an RNA sequence can be readily deduced from the DNA sequence.

TABLE 1
Sequences

| SEQ | | |
|---|---|---|
| ID | | |
| NO: | Description | Amino Acid or Nucleotide Sequence |
| 1. | aldehyde | MIKDTLVSITKDLKLKTNVENANLKNYKDDSSCFGVFENVENAISNAV |
| | dehydrogenase EutE | HAQKILSLHYTKEQREKIITEIRKAALENKEILATMILEETHMGRYED |
| | from Clostridium | KILKHELVAKYTPGTEDLTTTAWSGDNGLTVVEMSPYGVIGAITPSTN |
| | saccharoperbutylacetonicum | PTETVICNSIGMIAAGNTVVFNGHPGAKKCVAFAVEMINKAIISCGGP |
| | (GenBank | ENLVTTIKNPTMDSLDAIIKHPSIKLLCGTGGPGMVKTLLNSGKKAIG |
| | Accession No. | AGAGNPPVIVDDTADIEKAGKSIIEGCSFDNNLPCIAEKEVFVFENVA |
| | WP_015395720.1) | DDLISNMLKNNAVIINEDQVSKLIDLVLQKNNETQEYSINKKWVGKDA |
| | | KLFLDEIDVESPSSVKCIICEVSASHPFVMTELMMPILPIVRVKDIDE |
| | | AIEYAKIAEQNRKHSAYIYSKNIDNLNRFEREIDTTIFVKNAKSFAGV |
| | | GYEAEGFTTFTIAGSTGEGITSARNFTRQRRCVLAG |
| 2. | aldehyde | atgattaaagacacgctggtttctatcaccaaagatttaaaattaaaa |
| | dehydrogenase EutE | acaaatgttgaaaatgccaatctgaagaactacaaagatgattctagt |
| | from Clostridium | tgtttcggcgttttcgaaaatgttgaaaatgctatcagcaatgccgta |
| | saccharoperbutylacetonicum | cacgcacaaaagatattatcgctgcattatacaaaagaacaacgtgaa |
| | (GenBank | aaaatcatcactgagatacgtaaggccgcactggaaaataaagagatt |
| | Accession No. | ctggctacaatgattcttgaagaaacacatatgggacgttatgaagat |
| | CP004121.1) | aaaatattaaagcatgaactggtagctaaatacactcctgggacagaa |
| | | gatttaactactactgcctggagcggcgataacgggctgacagttgta |
| | | gaaatgtctccatatggcgttattggtgcaataactccttctaccaat |
| | | ccaactgaaactgtaatttgtaatagtattggcatgattgctgctgga |
| | | aatactgtggtatttaacggacatccaggcgctaaaaaatgtgttgct |
| | | tttgctgtcgaaatgatcaataaagctattattagctgtggtggtccg |
| | | gagaatctggtaacaactataaaaaatccaaccatggactctctggat |
| | | gccattattaagcacccttcaataaaactgctttgcggaactggcggg |
| | | ccaggaatggtaaaaaccctgttaaattctggtaagaaagctattggt |
| | | gctggtgctggaaatccaccagttattgtcgatgatactgctgatatt |
| | | gaaaaggctggtaagagtatcattgaaggctgttcttttgataataat |
| | | ttaccttgtattgcagaaaaagaagtatttgtttttgagaacgttgca |
| | | gatgatttaatatctaacatgctgaaaaataatgctgtaattatcaat |
| | | gaagatcaggtatcaaagttaatcgatttagtattacaaaaaaataat |
| | | gaaactcaagaatactctattaataagaaatgggtcggtaaagatgca |
| | | aaattattcctcgatgaaatcgatgttgagtctccttcaagcgttaaa |
| | | tgcattatctgcgaagtcagtgcaagccatccatttgttatgacagaa |
| | | ctgatgatgccaatattaccaattgtgcgcgttaaagatatcgatgaa |
| | | gctattgaatatgcaaaaattgcagaacaaaatagaaaacatagtgcc |
| | | tatatttattcaaaaaatatcgacaacctgaatcgctttgaacgtgaa |
| | | atcgatactactatctttgtaaagaatgctaaatcttttgccggtgtt |
| | | ggttatgaagcagaaggctttaccactttcactattgctggatccact |
| | | ggtgaaggcataacttctgcacgtaattttacccgccaacgtcgctgt |
| | | gtactggccggttaa |
| 3. | Variant 1 - | MIKDTLVSITKDLKLKTNVENANLKNYKDDSSCFGVFENVENAISNAV |
| | [includes | HAQKILSLHYTKEQREAMITEIRKAALENKEILATMILEETHMGRYED |
| | alterations K65A | KILKHELVAKYTPGTEDLTTTAWSGDNGLTVVEMSPYGVIGAITPSTN |
| | I66M C174S M204R | PTETVICNSIGMIAAGNTVVFNGHPGAKKSVAFAVEMINKAIISCGGP |
| | C220V A243Q C267A | ENLVTTIKNPTRDSLDAIIKHPSIKLLVGTGGPGMVKTLLNSGKKAIG |
| | C356T R396H E437P | AGQGNPPVIVDDTADIEKAGKSIIEGASFDNNLPCIAEKEVFVFENVA |
| | F442N C464I A467V | DDLISNMLKNNAVIINEDQVSKLIDLVLQKNNETQEYSINKKWVGKDA |
| | relative to SEQ ID | KLFLDEIDVESPSSVKCIITEVSASHPFVMTELMMPILPIVRVKDIDE |
| | NO: 1] pG10911 | AIEYAKIAEQNHKHSAYIYSKNIDNLNRFEREIDTTIFVKNAKSFAGV |
| | | GYEAPGFTTNTIAGSTGEGITSARNFTRORRIVLVG |
| 4. | Variant 1 - [encodes | atgattaaagacacgctggtttctatcaccaaagatttaaaattaaaa |
| | polypeptide with | acaaatgttgaaaatgccaatctgaagaactacaaagatgattctagt |
| | alterations K65A | tgtttcggcgttttcgaaaatgttgaaaatgctatcagcaatgccgta |
| | I66M C174S M204R | cacgcacaaaagatattatcgctgcattatacaaaagaacaacgtgaa |
| | C220V A243Q C267A | gcgatgatcactgagatacgtaaggccgcactggaaaataaagagatt |
| | C356T R396H E437P | ctggctacaatgattcttgaagaaacacatatgggacgttatgaagat |
| | F442N C464I A467V | aaaatattaaagcatgaactggtagctaaatacactcctgggacagaa |
| | relative to SEQ ID | gatttaactactactgcctggagcggcgataacgggctgacagttgta |
| | NO: 1] pG10911 | gaaatgtctccatatggcgttattggtgcaataactccttctaccaat |
| | | ccaactgaaactgtaatttgtaatagtattggcatgattgctgctgga |
| | | aatactgtggtatttaacggacatccaggcgctaaaaaatctgttgct |
| | | tttgctgtcgaaatgatcaataaagctattattagctgtggtggtccg |
| | | gagaatctggtaacaactataaaaaatccaaccagggactctctggat |
| | | gccattattaagcacccttcaataaaactgcttgttggaactggcggg |
| | | ccaggaatggtaaaaaccctgttaaattctggtaagaaagctattggt |
| | | gctggtcagggaaatccaccagttattgtcgatgatactgctgatatt |
| | | gaaaaggctggtaagagtatcattgaaggcgcttcttttgataataat |
| | | ttaccttgtattgcagaaaaagaagtatttgtttttgagaacgttgca |
| | | gatgatttaatatctaacatgctgaaaaataatgctgtaattatcaat |
| | | gaagatcaggtatcaaagttaatcgatttagtattacaaaaaaataat |
| | | gaaactcaagaatactctattaataagaaatgggtcggtaaagatgca |
| | | aaattattcctcgatgaaatcgatgttgagtctccttcaagcgttaaa |
| | | tgcattatcactgaagtcagtgcaagccatccatttgttatgacagaa |
| | | ctgatgatgccaatattaccaattgtgcgcgttaaagatatcgatgaa |
| | | gctattgaatatgcaaaaattgcagaacaaaatcataaacatagtgcc |
| | | tatatttattcaaaaaatatcgacaacctgaatcgctttgaacgtgaa |
| | | atcgatactactatctttgtaaagaatgctaaatcttttgccggtgtt |
| | | ggttatgaagcaccgggctttaccactaatactattgctggatccact |
| | | ggtgaaggcataacttctgcacgtaattttacccgccaacgtcgcatt |
| | | gtactggtcggttaa |

It is understood that modifications which do not substantially affect the activity of the various embodiments of this invention are also provided within the definition of the invention provided herein. Accordingly, the following examples are intended to illustrate but not limit the present invention.

### Example I

### Identification of Aldehyde Dehydrogenase Variants

This example describes generation of aldehyde dehydrogenase variants with desirable properties. The design strategy included using an aldehyde dehydrogenase crystal structure for structure-based design of the enzyme active site, where the crystal structure contained (R)-3-hydroxybutyryl-CoA (R-3-HB-CoA) bound to the catalytic cysteine, C275. This intermediate was used to select sites for mutagenesis, around the active-site at a fixed distance. With this structural model of the aldehyde dehydrogenase structure, a library of variants was generated using site-directed mutagenesis of all 20 amino acids at each site using Variant 1 (SEQ ID NO: 3) as the template.

In order to examine the activity of the aldehyde dehydrogenase variants that were generated, an in vitro assay as described in Example IV was performed. In particular, assays were performed with 3HB-CoA or acetyl-CoA (AcCoA) as a substrate, and improved aldehyde dehydrogenase variants were identified as those variants showing an improvement in the ratio of activity for 3HB-CoA vs. AcCoA. Aldehyde dehydrogenase variants that have increased specificity of 3HB-CoA over AcCoA have the advantage of providing a decrease in ethanol, since the acetaldehyde generated from AcCoA can be converted to ethanol by enzymes natively in the host cell or by a pathway enzyme that converts 3-hydroxybutyraldehyde to 1,3-butanediol.

The results of these assays were used to guide the next set of aldehyde dehydrogenase variant designs, where variants showing higher lysate activity and/or substrate specificity were selected for Sangar sequencing to identify alterations responsible for the improvements in either activity of the desired substrate, R-3HB-CoA, and/or specificity of R-3HBCoA/AcCoA. After sequencing, a set of these mutations were combined to yield additional variants generated by site-directed mutagenesis. This library of the variants were assayed in vitro.

### Example II

### In Vitro Lysate Activity Assay

In order to evaluate the impact of each selected aldehyde dehydrogenase variant identified by the methods described in Example I, an in vitro lysate activity assay was conducted. An E. coli strain containing a plasmid having a nucleotide sequence encoding an aldehyde dehydrogenase variant on a constitutive promoter was generated. The strain was inoculated in LB with carbenicillin (100 μg/mL) and grown overnight at 35° C. in a shaking incubator. The overnight culture was diluted into fresh LB with carbenicillin grown overnight at 35° C. in a shaking incubator. Cells were collected by centrifugation and frozen at −20° C. until the day of conducting an in vitro lysate assay.

For the in vitro lysate assay, the cell pellet was thawed and resuspended in 0.1 M Tris-HCl, pH 7.0 buffer. The OD600 was measured for the cell suspension and each of the test variants were normalized to an OD of 4. Pellets were prepared by centrifugation and the pellet was then lysed with a chemical lysis reagent containing 10 mM DTT, nuclease, and lysozyme for 30 minutes at room temperature. This lysate was used to measure the activity of an aldehyde dehydrogenase variant to catalyze the conversion of (R)-3-hydroxybutyryl-CoA (R-3-HB-CoA) to (R)-3-hydroxybutyraldehyde (R-3-HBal) at 35° C. by an enzyme-coupled assay. The enzyme-coupled assay included a purified form of a recombinant CoA-ligase from Ruegeria pomeroyi for converting (R)-3-hydroxybutyrate (R-3-HIB) to R-3-HB-CoA and the test lysate having the aldehyde dehydrogenase variant for converting R-3-HB-CoA to R-3-HBal. The standard assay solution contained an aliquot of the lysate, 1 μM CoA-ligase, 5 mM R-3-HJB, 5-10 μM CoA, 1 mM ATP, and 5 mM MgCl₂, and 0.3 mM NADH were mixed in 0.04 mL of 0.1 M Tris-HCl, pH 7.4 buffer. The kinetics of the reaction was monitored by measuring the linear decrease in cofactor fluorescence (excitation wavelength=360 nm; emission wavelength=465 nm) using a microtiter plate reader. Alternatively, to measure lysate activity on acetyl-CoA (AcCoA), the above solution was replaced with 25 mM acetate.

The results of these screens, including identifying the activity of select variants, are shown in TABLE 2.

TABLE 2
Exemplary Aldehyde Dehydrogenase Variants Engineered using the
Variant 1 (SEQ ID NO: 3) as the Template with Positional Alteration(s)
and Corresponding Activity on Select Substrates

| | | | | R-3-HB- | R-3-HB-CoA/ |
|---|---|---|---|---|---|
| Variant # | Residue | Position | Alteration | CoA | AcCoA Ratio |
| 1 | — | — | — | + | ND |
| 2 | Q | 243 | E | + | ND |
| 3 | A | 277 | C | + | ND |
| 4 | E | 435 | H | + | ND |
| 5 | E | 435 | Q | + | ND |
| 6 | E | 435 | M | + | ND |
| 7 | E | 435 | R | + | ND |
| 8 | S | 142 | V | + | ND |
| 9 | S | 142 | I | + | ND |
| 10 | T | 146 | C | + | ND |
| 11 | Y | 401 | F | + | ND |
| 12 | N | 442 | F | + | ND |

R-3-HB-CoA (variant activity/Variant 1 activity): + = 0-1.2
R-3-HB-CoA/AcCoA Ratio: ND = no AcCoA detectable

### Example III

### Small Scale In Vivo Assay

In order to evaluate the impact of each selected aldehyde dehydrogenase variant identified by the method described in Example I, a small scale in vivo activity assay was conducted. A plasmid containing a gene encoding a variant was transformed into a AAld strain of E. coli that also included introduced genes encoding 1,3-BDO pathway enzymes: 1) a thiolase (Thl), 2) a 3-hydoxybutryl-CoA dehydrogenase (Hbd), 3) an alcohol dehydrogenase (Adh), and 4) NAD-utilizing formate dehydrogenase (Fdh). Alternatively, the gene encoding a variant was integrated onto the chromosome of an E. coli strain already expressing the same 1,3-BDO pathway enzymes. The 3-hydoxybutryl-CoA dehydrogenase utilizes NADH as a cofactor.

The resulting strains were tested for 1,3-BDO production. The engineered E. coli cells were fed 2% glucose in minimal media, and after a 24 hr incubation at 35° C., the cells were harvested, and the supernatants were evaluated by analytical HPLC or standard LC-MS analytical method for 1,3-BDO levels.

The results of these screens, including identifying the activity of select variants, are shown in TABLE 3.

TABLE 3
Exemplary Aldehyde Dehydrogenase Variants Engineered
using the Variant 1 (SEQ ID NO: 3) as the Template
with Positional Alteration(s) and Corresponding
Activity Leading to Production of 1,3-BDO

| | | | | 1,3-BDO |
|---|---|---|---|---|
| Variant # | Residue | Position | Alteration | Production |
| 1 | — | — | — | + |
| 2 | Q | 243 | E | ND |
| 3 | A | 277 | C | ND |
| 4 | E | 435 | H | ND |
| 5 | E | 435 | Q | ND |
| 6 | E | 435 | M | + |
| 7 | E | 435 | R | + |
| 8 | S | 142 | V | ND |
| 9 | S | 142 | I | ND |
| 10 | T | 146 | C | ND |
| 11 | Y | 401 | F | ND |
| 12 | N | 442 | F | + |

1,3-BDO Production: + = detectable 1,3-BDO production
ND = not detectable

Throughout this application various publications have been referenced. The disclosures of these publications in their entireties are hereby incorporated by reference in this application in order to more fully describe the state of the art to which this invention pertains. Although the invention has been described with reference to the examples provided above, it should be understood that various modifications can be made without departing from the spirit of the invention.

What is claimed is:

1. An engineered aldehyde dehydrogenase comprising a variant of amino acid sequence SEQ ID NO: 3 or a functional fragment thereof, wherein the engineered aldehyde dehydrogenase comprises one or more amino acid alterations at a position described in TABLE 2.

2. The engineered aldehyde dehydrogenase of claim 1, wherein the engineered aldehyde dehydrogenase:
(i) is capable of catalyzing the conversion of 3-hydroxybutyryl-CoA to 3-hydroxybutyraldehyde,
(ii) has higher specificity for conversion of 3-hydroxybutyryl-CoA to 3-hydroxybutyraldehyde over conversion of acetyl-CoA to acetaldehyde;
(iii) has higher specificity for conversion of (R)-3-hydroxybutyryl-CoA to (R)-3-hydroxybutyraldehyde over conversion of (S)-3-hydroxybutyryl-CoA to (S)-3-hydroxybutyraldehyde:
(iv) is capable of catalyzing the conversion of 4-hydroxybutyryl-CoA to 4-hydroxybutyraldehyde;
(v) has higher specificity for conversion of 4-hydroxybutyryl-CoA to 4-hydroxybutyraldehyde over conversion of acetyl-CoA to acetaldehyde; and/or
(vi) comprises an activity that is at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, or at least 200% higher than the activity of an aldehyde dehydrogenase consisting of the amino acid sequence of SEQ ID NO: 3.

3.-7. (canceled)

8. The engineered aldehyde dehydrogenase of claim 1, wherein the engineered aldehyde dehydrogenase comprises one or more amino acid alterations at a position corresponding to position 142, 243, 277, 401, 435, or 442, or a combination thereof, in SEQ ID NO: 3.

9.-11. (canceled)

12. The engineered aldehyde dehydrogenase of claim 1, wherein the one or more amino acid alterations result in an engineered aldehyde dehydrogenase comprising:
a) I or V at a residue corresponding to position 142 in SEQ ID NO: 3;
b) C at a residue corresponding to position 146 in SEQ ID NO: 3;
c) E at a residue corresponding to position 243 in SEQ ID NO: 3;
d) C at a residue corresponding to position 277 in SEQ ID NO: 3;
e) F at a residue corresponding to position 401 in SEQ ID NO: 3;
f) H, M, Q, or R at a residue corresponding to position 435 in SEQ ID NO: 3; and/or
g) F at a residue corresponding to position 442 in SEQ ID NO: 3.

13. The engineered aldehyde dehydrogenase of claim 1, wherein the one or more amino acid alterations comprises:
(i) an amino acid residue F corresponding to position 442 in SEQ ID NO: 3; and
(ii) an amino acid alteration at position 142 or 243 in SEQ ID NO: 3, or a combination thereof.

14. The engineered aldehyde dehydrogenase of claim 1, wherein the one or more amino acid alterations comprise at least 2, 3, 4, 5, 6, or 7 alterations and/or wherein the amino acid sequence has at least 65% sequence identity to the amino acid sequence referenced in SEQ ID NO: 3.

15.-23. (canceled)

24. The engineered aldehyde dehydrogenase of claim 1, wherein the amino acid sequence, other than the one or more amino acid alterations, is identical to the amino acid sequence referenced in SEQ ID NO: 3.

25. A recombinant nucleic acid encoding the engineered aldehyde dehydrogenase of claim 1.

26. (canceled)

27. (canceled)

28. A non-naturally occurring microbial organism comprising a recombinant nucleic acid encoding an engineered aldehyde dehydrogenase claim 1.

29. The non-naturally occurring microbial organism of claim 28, wherein the non-naturally occurring microbial organism:
(i) further comprises a pathway that produces 3-hydroxybutyraldehyde and/or 1,3-butanediol, or an ester or amide thereof;
(ii) is capable of producing at least 10% more 3-hydroxybutyraldehyde and/or 1,3-butanediol, or an ester or amide thereof compared to a control microbial organism that does not comprise the recombinant nucleic acid encoding the engineered aldehyde dehydrogenase;
(iii) further comprises a pathway that produces 4-hydroxybutyraldehyde and/or 1,4-butanediol, or an ester or amide thereof;
(iv) is capable of producing at least 10% more 4-hydroxybutyraldehyde and/or 1,4-butanediol, or an ester or amide thereof compared to a control microbial organism that does not comprise the recombinant nucleic acid encoding the engineered aldehyde dehydrogenase;
(v) produces a decreased amount of a by-product as compared to a control microbial organism that does not comprise the recombinant nucleic acid encoding the engineered aldehyde dehydrogenase, wherein optionally the by-product is ethanol or 4-hydroxy-2-butanone:
(vi) is capable of producing at least 10% less by-product compared to a control microbial organism that does not comprise the recombinant nucleic acid therecombinant nucleic acid encoding the engineered aldehyde dehydrogenase,
(vii) is in a substantially anaerobic culture medium; and/or
(viii) is a species of bacteria, yeast, or fungus.

30.-32. (canceled)

33. The non-naturally occurring microbial organism of claim 29, wherein the one or more enzymes of the pathway are encoded by an exogenous nucleic acid.

34.-41. (canceled)

42. A method for producing 3-hydroxybutyraldehyde and/or 1,3-butanediol, or an ester or amide thereof, comprising culturing the non-naturally occurring microbial organism of claim 28 under conditions and for a sufficient period of time to produce the 3-hydroxybutyraldehyde and/or 1,3-butanediol, or an ester or amide thereof.

43.-44. (canceled)

45. A culture medium comprising the 3-hydroxybutyraldehyde and/or 1,3-butanediol, or an ester or amide thereof produced by the method of claim 42, wherein the 3-hydroxybutyraldehyde and/or 1,3-butanediol, or an ester or amide thereof has a carbon-12, carbon-13 and carbon-14 isotope ratio that reflects an atmospheric carbon dioxide uptake source.

46. A 3-hydroxybutyraldehyde and/or 1,3-butanediol, or an ester or amide thereof produced according to the method of claim 42.

47.-49. (canceled)

50. A composition comprising the 3-hydroxybutyraldehyde and/or 1,3-butanediol, or an ester or amide thereof of claim 46, or a cell lysate or culture supernatant thereof.

51. A method for producing 4-hydroxybutyraldehyde and/or 1,4-butanediol, or an ester or amide thereof, comprising culturing the non-naturally occurring microbial organism of claim 29 under conditions and for a sufficient period of time to produce the 4-hydroxybutyraldehyde and/or 1,4-butanediol, or an ester or amide thereof.

52. (canceled)

53. (canceled)

54. A culture medium comprising the 4-hydroxybutyraldehyde and/or 1,4-butanediol, or an ester or amide thereof produced by the method of claim 51, wherein the 4-hydroxybutyraldehyde and/or 1,4-butanediol, or an ester or amide thereof has a carbon-12, carbon-13 and carbon-14 isotope ratio that reflects an atmospheric carbon dioxide uptake source.

55. A 4-hydroxybutyraldehyde and/or 1,4-butanediol, or an ester or amide thereof produced according to the method of claim 51.

56.-58. (canceled)

59. A composition comprising the 4-hydroxybutyraldehyde and/or 1,4-butanediol, or an ester or amide thereof of claim 55, or a cell lysate or culture supernatant thereof.

60. (canceled)

61. A composition comprising the engineered aldehyde dehydrogenase of claim 1 and at least one substrate for the engineered aldehyde dehydrogenase.

62.-65. (canceled)
